(19) United States
(12) Patent Application Publication
(10) Pub. No.: US 2012/0207690 A1
(43) Pub. Date: Aug. 16, 2012

(54) Searching methods using genetic responsivity measurements

(76) Inventors: Lawrence R. Weill, Seal Beach, CA (US); Edward F. Tuck, West Covina, CA (US); Mark A. Sturza, Encino, CA (US); Martie G. Haselton, Culver City, CA (US); Christine E. Garver-Apgar, Louisville, CO (US); Thomas N. Giaccherini, Carnel Valley, CA (US)

(21) Appl. No.: 13/373,366

(22) Filed: Jun. 7, 2011

Related U.S. Application Data
(63) Continuation-in-part of application No. 11/881,153, filed on Jul. 24, 2007, now abandoned.
(60) Provisional application No. 60/834,025, filed on Jul. 28, 2006.

(51) Int. Cl. A61K 8/64; A61Q 1/06; G06F 19/22; A61K 38/02

(52) U.S. Cl. 424/64; 514/1.1; 702/19; 702/20

(57) ABSTRACT

Methods and apparatus for using an energy emanating device that finds a person (17a,b) object or system based on preselected attributes (33) stored in the energy emanating device (10) are disclosed. Searching Methods Using Genetic Responsivity Measurements are used to compare the attributes (33) of individuals, and a match is determined based upon the correlation of these attributes (33). The matching is accomplished using a variety of algorithms, including a “Genetic Responsivity Measurement Formula.” In alternative embodiments, the invention may be used in a search engine.

### CROSS-REFERENCES TO RELATED PENDING U.S. PATENT APPLICATIONS & CLAIMS FOR PRIORITY

This Continuation-in-Part patent application is related to the following U.S. patent applications:

| | |
|---|---|
| U.S.S.N. 60/834,025 | 28 Jul. 2006 |
| U.S.S.N. 11/239,603 | 28 Sep. 2005 |
| U.S.S.N. 11/286,143 | 23 Nov. 2005 |
| U.S.S.N. 11/360,025 | 21 Feb. 2006 |
| U.S.S.N. 11/405,001 | 14 Apr. 2006 |
| U.S.S.N. 11/881,153 | 24 Jul. 2007 |
| U.S.S.N. 12/290,877 | 3 Nov. 2008 |
| U.S.S.N. 12/313,263 | 17 Nov. 2008 |
| U.S.S.N. 12/590,433 | 5 Nov. 2009 |
| U.S.S.N. 12/590,515 | 24 Oct. 2008 |
| U.S.S.N. 12/799,210 | 19 Apr. 2010 |

The Applicants hereby claim priority for any subject matter that is commonly disclosed in the U.S. Provisional and Non-Provisional Applications identified in this paragraph, and in this Continuation-in-Part patent application.

### FIELD OF THE INVENTION

The present invention pertains to the field of predicting good matches for individuals. More particularly, one embodiment of the invention furnishes relationship predictions directly to customers, while another embodiment supplies relationship predictions to a company which offers online dating or other dating or introduction services.

One embodiment of the present invention pertains to methods and apparatus for using an electronic device to find a person or system that meets criteria specified by a user and/or to establish mutual compatibility between or among two or more people or systems. More particularly, one preferred embodiment of the invention uses a small radiating device which utilizes radio, optical, ultrasonic or other means that automatically and continuously or periodically emits a signal which interrogates other similar devices.

When the user's device finds another person or system whose device returns a signal that matches the user's pre-specified criteria, the user is alerted by a visual and/or audible signal. The matching is accomplished using a variety of algorithms, including a “Genetic Responsivity Measurement Formula.”

In another embodiment of the invention, the Genetic Responsivity Measurement Formula is incorporated into a search engine, and is used to find information, a product or a replacement part on the Internet, or in some other database.

### BACKGROUND OF THE INVENTION

I. The Biology of Matching

Mammals have evolved efficient ways to find and select among potential mates. There has been a great deal of research on this subject in the twenty-three years since a landmark study found that mice choose their mates on the basis of their candidates' distinctive odors. Boyse, E. A.; Beauchamp, G. K.; Yamazaki, K; et al., “Chemosensory Communication—A New Aspect of the Major Histocompatibility Complex and Other Genes in the Mouse.” Journal: Oncodevelopmental Biology and Medicine. Vol. 4 No. 1-2: Pages 101-116, 1982. These odors are defined by the Major Histocompatibility Complex (MHC). The MHC is a cluster of genes that determines details of cellular surfaces and thus immune responses, and specifies certain peptides that appear in skin secretions and urine. These peptides are responsible for odors which uniquely identify individuals who are not identical twins.

More recent work has shown that human female sexual responsivity to a male partner varies linearly and inversely with the degree to which genes in the Major Histocompatibility Complex are shared. Garver-Apgar, Christine E. et al., “MHC Alleles, Sexual Responsivity, and Unfaithfulness in Romantic Couples,” Psychological Science, Volume 17, Number 10, (October 2006). The correspondence is dramatic: about a nine (on a self-reported scale of one to ten) in responsivity to men who share none of a woman's MHC genes and to those who share sixty percent.

Men and women detect others' MHC genes through their body odors. There are a number of peptides that are derived from particular regions of the MHC. These peptides are detected as odors. They strongly affect a woman's responsivity to a particular partner, as discussed in the cited literature, and to both men's and women's mutual attractiveness.

This mate-selection process has a strong effect on the fitness of offspring. Choosing mates on the basis of MHC dissimilarity equips offspring with a broad immune system, increasing the offspring's fitness, and also reduces the rate of spontaneous abortion. It also selects against close relatives as mates, increasing the viability of fetuses and reducing birth defects. It also reduces the rate of spontaneous abortion: there is compelling evidence that fetuses of couples which share significant numbers of MHC alleles are more likely to be rejected in utero. Komlos, L., Zamir, R., Joshua, H., and Halbrecht, I., “Common HLA Antigens in Couples with Repeated Abortions,” Clinical Immunology and Immunopathology 7, Pages 330-335 (1977).

Other studies, including one cited above, have shown that women who are in long-term intimate relationships with men with similar MHC alleles are more likely to report being attracted to and fantasizing about other partners during the fertile portion of their menstrual cycles. This practice obviously has a destabilizing effect on these relationships, which include marriages. Because humans' sense of smell is relatively poor, couples who are strangers must come into close personal contact before they can estimate their MHC-derived “fit” with a potential male partner and thus a woman's long-term sexual responsivity to her partner. As humans have moved from villages to cities, various means have been created to bring men and women of marriageable age into close proximity under controlled conditions: examples range from the masked ball in Romeo and Juliet to modern on-line dating services. In modern human society, with much less class structure and much more freedom for men and women than in tribal, medieval or Victorian eras, and a much higher probability of encountering strangers than in primitive (pre-tribal) eras, this acquaintance process can pose considerable danger and risk of embarrassment to women. The modern process of selecting a mate is very inefficient compared to these earlier societies, in which the number of potential partners available to each woman was comparatively small, and in primitive societies where people lived in very close proximity. It would be of great benefit, not only to individual couples, but to society as a whole, if men and women could assess their sexual compatibility and the health of any offspring of the union without coming into close contact. This would, among other things, give women a wider range of prequalified candidates and would give men greater assurance that they and their prospective mates would have a stable and persistent relationship characterized by mutual physical attraction. It is generally conceded that mutual sexual attraction and responsivity are major contributors to pair bonding: they are the glue that holds long-term relationships together. People of all political and religious persuasions agree that stable pair-bonding, carrying the benefit of reduced strife and relationship discord, is in the best interest of society. Strife and relationship discord result in failed marriages and in infidelity. Society as a whole will thus benefit from easier and more accurate responsivity assessment. It is also important to note that there remain many cultures in which arranged marriages are the norm, and in which affianced couples do not meet before their wedding ceremony. Parents and matchmakers who are concerned with the success of their efforts could gain confidence from an MHC-based genetic matching process before a commitment is made.

Technology has advanced to the point that individual MHC-derived peptides, and thus odors, can be accurately detected artificially using gas chromatography and/or mass spectrometry (an “e-nose”). Willse, Alan et al., “Identification of Major Histocompatibility Complex-Regulated Body Odorants by Statistical Analysis of a Comparative Gas Chromatography/Mass Spectrometry Experiment,” Analytical Chemistry, Vol. 77, No. 8 (Apr. 15, 2005). This implies that a personal odor profile can be constructed for each individual, and that the degree of MHC-allele sharing of two individuals can be derived by comparing those measurements, even if they are strangers and geographically distant from one another. MHC analysis can also be done on the basis of other material, such as cheek-cell scrapings, saliva tests, and other means used in forensic settings.

This process represents a considerable improvement to acquaintance-facilitation (“dating”) services based on the use of questionnaires and personality profiling. While these services help people find partners based on their subjective preferences and personality match, they say little about the likelihood of sexual attraction on first meeting, or the sexual responsivity of the partners in a long-term relationship. In contrast to these methods, MHC comparison is a completely objective process. Unlike current processes which rely on self-administered questionnaires, remote psychological assessments and other user-supplied personal data, MHC comparison cannot misrepresent its user.

II. The Current Market for Matching Services & the Need for Improvement

According to Jupiter Research, online personals revenues will have risen from about four hundred million dollars in 2003, to over six hundred million dollars in 2009. In the United States alone, over seventeen million persons participate in online dating each year (Nielsen Media Research). In 2008, revenue from online dating services will exceed revenue from dating services and personal ads which appear in conventional media (Marketdata Enterprises, Inc.).

Despite this dramatic growth in the online dating industry, many individuals who have used online dating services remain disappointed with their results. The development of a system that provides a tool for predicting good matches based upon applied biological and genetic mechanisms of attraction would fulfill a long felt need in the dating and relationship industry, and would constitute a great benefit to members of society.

III. Internet Dating Services

Over the past decade, a variety of Internet dating services have been established. These services, such as Match.com™ or E-harmony.com™, offer on-line presentations of information and photos of individuals. None of these services furnishes a device, method or system for matching individuals who are simultaneously present within a pre-determined physical region. The development of a device or system that could help individuals find friends or mates in real time within a pre-determined physical region would constitute a major technological advance, and would satisfy long-felt needs and aspirations of the Internet dating industry and of Humanity in general.

Conventional search engines such those provided by Google™ and Yahoo!® are based on techniques that involve the popularity of webpages. These types of searches may sometimes fail to provide focused search results. The development of a method that could offer Internet or other database searches that furnish focused and accurate results would constitute a major technological advance, and would satisfy long-felt needs and aspirations of the information and computing industries.

### SUMMARY OF THE INVENTION

The present invention comprises methods and apparatus for providing a relationship prediction based on the correlation of personal characteristics and the analysis of genetic characteristics. In one embodiment, a first person supplies descriptions of his or her own personal characteristics, as well as descriptions of the personal characteristics of her or his ideal match, to a website; or by other means to a dating or introduction service provider. The first person also furnishes an odor, tissue or fluid sample to a test facility, where genetic characteristics are analyzed and determined. A relationship match is then generated based on both a combination of both a positive correlation of the personal characteristics of the first person and second person, and a measured dissimilarity between the sequence of genetic characteristics of the first person and second person. In one embodiment, relationship matches are provided directly to customers. In an alternative embodiment, relationship matches are provided to a company which offers online dating or other dating or introduction services. In another embodiment of the invention, a match is computed for a woman based upon her responsivity to a man, which is based upon the extent of dissimilarity of their MI-IC-alleles; and a correlation of the attributes and preferences of that woman and that man. In yet another embodiment, the customer may purchase a custom-formulated perfume, cologne, salve or other cosmetic or preparation that contains enhanced aromas that match his or her, or his or her partner's, genetic attributes.

### I. A First Group of Embodiments:

### Using Searching Methods Using Genetic Responsivity Measurements to Find a Match

One embodiment of the present invention provides a simple miniaturized electronic device that enables individuals to find a friend, a mate or someone with a specific interest or skill. In one embodiment of the invention, a man or a woman may program a MateFinder™ to help find an ideal match. In one particular embodiment, the MateFinder™ comprises a radio and a microprocessor with a non-volatile memory, such as a static RAM. Information that describes both the user and the ideal match can be written to the non-volatile memory. The radio automatically and periodically broadcasts a “seeking signal” over a short range. When the seeking signal is received by another MateFinder™, it is analyzed to determine the degree of correlation with the receiver's preferences. If the degree of correlation exceeds a preset minimum, the sender, the receiver, or both are alerted.

One embodiment of the invention uses the present invention to compare the attributes of individuals, and to then determine a good match based on the comparison of the attributes. In an alternative embodiment, the Applicants' present invention may be utilized without a MateFinder™ to determine a good match when used in combination with an Internet dating service.

### II. A Second Group of Embodiments:

### Using Searching Methods Using Genetic Responsivity Measurements as Part of an Internet Search Engine

A second embodiment of the present invention offers a search tool for a database, such as the Internet. This method uses the attributes of the target of the search, rather than a search that is based on “page-ranking” searches that conduct searches based on the previous popularity of all entries in a database that are available to the searcher.

### III. A Third Group of Embodiments:

More Advanced Searching Methods Using Genetic Responsivity Measurements with Additional User Inputs & Controls

In a third embodiment, additional controls and inputs are provided for the user to optimize his or her search.

### IV. A Fourth Group of Embodiments:

A fourth embodiment of the invention may be used to create a website for finding products or parts in situations when a keyword search is not helpful.

### V. A Fifth Group of Embodiments:

In a fifth embodiment, various methods for predicting a good match using genetic attributes are described.

An appreciation of the other aims and objectives of the present invention, and a more complete and comprehensive understanding of this invention, may be obtained by studying the following description of preferred and alternative embodiments, and by referring to the accompanying drawings.

### A BRIEF DESCRIPTION OF THE DRAWINGS

FIG. 1 offers a view of men and women attending a party. The man has a MateFinder™ clipped to his belt. Some of the women at the party have a MateFinder™ clipped to a purse or belt, or concealed in their clothing. The man's MateFinder™ produces a short-range radio signal that interrogates other MateFinders™ that are nearby. The interrogation signal may broadcast a description of the man and seeks another MateFinder™ that stores a list of qualities, characteristics or criteria that describe the gentleman's aspirations, preferences or requirements in a mate. When the interrogation signal finds a good match, the man is alerted by a visual or audible alert. If the man's own attributes match those sought by one of the women, the MateFinder™ may alert her to the presence of the man.

Various embodiments of the MateFinder™ are disclosed in U.S. Ser. No. 11/239,603 and U.S. Ser. No. 11/286,243.

FIG. 2 supplies a perspective view of one particular embodiment of the invention, which may be clipped to a man's belt or to a woman's purse, or which may be concealed in the clothing of either. The MateFinder™ may also be incorporated into a bracelet, watch, necklace, tietack, shoe, hat or some other fashionable item or article.

FIG. 3 illustrates a woman who has visited a website using her personal computer. In this example, the website is located at www.e-pheromone.com. She has connected her MateFinder™ device to the USB port of her computer, and is programming her MateFinder™ with her own attributes, as well as with her preferences in a mate.

FIG. 4 is a flowchart that explains how a user employs the MateFinder™ to help find a person with characteristics that match the user's preferences.

FIG. 5 shows the same woman at some sort of social event, perhaps at the beach, in a park, at a concert or attending a sporting event. The woman's MateFinder™ has found a match in the crowd, and alerts her to his presence.

FIG. 6 exhibits the operation of a pair of MateFinders™. When the man's device finds a woman's MateFinder™ that presents a good match, his device alerts him. In an alternative embodiment, the woman may respond by using her cellular telephone to send a message back to her prospective match, or by using a text-message feature of her MateFinder™.

FIG. 7 is a simplified schematic diagram of one embodiment of the present invention.

FIG. 8 is a more detailed schematic diagram which exhibits a particular implementation of the present invention.

FIG. 9 is a flowchart that outlines a basic design for a software program that may be utilized in one embodiment of the invention.

FIGS. 10, 11, 12 and 13 are flowcharts which illustrate correlation methods that may be used to implement the present invention.

FIG. 14 is an abstract representation of a database or collection of information, such as the Internet.

FIG. 15 is a map which shows the sources of information in a master set and each of the selected attributes.

FIG. 16 presents a Table of Shortest Genetic Responsivity Measurements, which is created by selecting the shortest Genetic Responsivity Measurement that has been measured between one attribute and one source for each attribute. The shortest Genetic Responsivity Measurement signifies the highest relevance.

FIG. 17 exhibits the combination of Searching Methods Using Genetic Responsivity Measurements with a conventional page-rank search.

FIG. 18 depicts a flow chart for one specific matching algorithm.

FIG. 19 depicts a flow chart for an alternative matching algorithm.

FIG. 20 shows a woman using a personal computer to sign up for an account with a Big Internet Dating Service, and then place an order for an AromaMatch™ Test Kit.

FIG. 21 shows a page from the Big Internet Dating Service website that may be used to place an order.

FIG. 22 shows a page from the Big Internet Dating Service that may be used to enter attributes about the customer and his or her ideal match.

FIG. 23 shows an alternative embodiment of the invention, in which the woman uses a telephone to open the account and to place the order.

FIG. 24 shows a woman purchasing an AromaMatch™ Test Kit at a retail store.

FIG. 25 shows a woman receiving an AromaMatch™ Test Kit in a doctor's office.

FIG. 26 shows a woman receiving an AromaMatch™ Test Kit from a church or some other religious organization.

FIG. 27 depicts the woman opening the AromaMatch™ Test Kit to reveal its contents: a bottle, a cotton ball, a sample patch, a sealable plastic bag and a mailing envelope.

FIG. 28 supplies a detailed view of the sample patch.

In FIG. 29, the woman cleans a patch of skin in preparation for applying the sample patch to her armpit.

In FIG. 30, the woman applies the patch to her armpit.

FIG. 31 shows the woman wearing the patch for a day or longer.

FIG. 32 portrays the woman removing the sample patch from her arm on the next day.

In FIG. 33, the woman places the sample patch that she has worn for a day into the bag, and seals it.

In FIG. 34, the woman writes her password on the sealable bag.

In FIG. 35, the sample that has been sealed in the bag is placed in a mailing envelope.

FIG. 36 shows the woman mailing an envelope which contains the bag, which, in turn, contains the worn sample patch.

FIG. 37 shows a laboratory technician using an analyzer to determine the genetic attributes of the odor sample that has been received from the woman depicted in FIG. 36.

In FIG. 38, the woman uses her computer to visit a website to obtain the results of the laboratory analysis.

In FIG. 39, the website reports the results of a matching process that has been performed using a library of candidates.

FIG. 40 shows the woman receiving test results from a postal worker.

FIG. 41 shows the woman receiving test results from a physician.

FIG. 42 an alternative embodiment of the invention, in which a tissue sample is obtained using a cheek swab.

FIG. 43 exhibits yet another alternative embodiment, which collects a sample directly from the air surrounding a man.

FIG. 44 depicts the collection of a saliva sample in a container.

In FIG. 45, the sample saliva is mixed.

FIG. 46 shows the sample's being placed in a sample bag.

FIG. 47 shows the sample bag's being placed in a mailing box.

FIG. 48 shows the mailing box's being sealed.

FIG. 49 shows the box's being mailed.

FIG. 50 is a flow chart which illustrates laboratory collection kit preparation tasks.

FIG. 51 is a flow chart which illustrates dating service tasks.

FIG. 52 is a flow chart which illustrates customer tasks.

FIG. 53 is a flow chart which illustrates laboratory analysis, matching and reporting tasks.

FIG. 54 is a flow chart which illustrates dating service and laboratory cooperative tasks.

FIG. 55 is a graph of MHC alleles shared on the horizontal axis, a woman's sexual responsivity to partner on the vertical axis.

FIG. 56 is a bar chart showing the number of MHC alleles shared on the horizontal axis, and the woman's expected sexual responsivity to her partner on the vertical axis.

FIG. 57 is a chart that shows the relationship of alleles in the MHC Group on Human Chromosome No. 6.

FIG. 58 reveals the details of the MHC Allele Groups.

FIG. 59 illustrates HLA-A Allele Group Frequency for a European Population Dataset.

FIG. 60 illustrates HLA-B Allele Group Frequency for a European Population Dataset.

FIG. 61 illustrates HLA-DR [symbol] 1 Allele Group Frequency for a European Population Dataset.

FIG. 62 depicts Allele Group Frequencies.

FIG. 63 depicts A/B/DR [symbol] 1 Group Haplotype Frequency.

FIG. 64 shows a man using a MateFinder™ device.

FIG. 65 provides a more detailed view of a MateFinder™ device.

In FIG. 66, a woman whose tissue sample has already been analyzed receives a custom-formulated perfume which contains aromas that correspond to her genetic attributes.

FIG. 67 depicts a method of manufacturing a customized perfume.

FIG. 68 presents a Genoscope™ graphical aid, which may be used to indicate good or bad matches.

### DETAILED DESCRIPTION OF PREFERRED & ALTERNATIVE EMBODIMENTS

### I. A First Group of Embodiments:

### Using Searching Methods Using Genetic Responsivity Measurements to Find a Match

The present invention comprises methods and apparatus for finding someone or something with specific attributes using a radio device. In one embodiment of the invention, a MateFinder™ 10, which resembles a pager, may be used by a man or a woman to find a match.

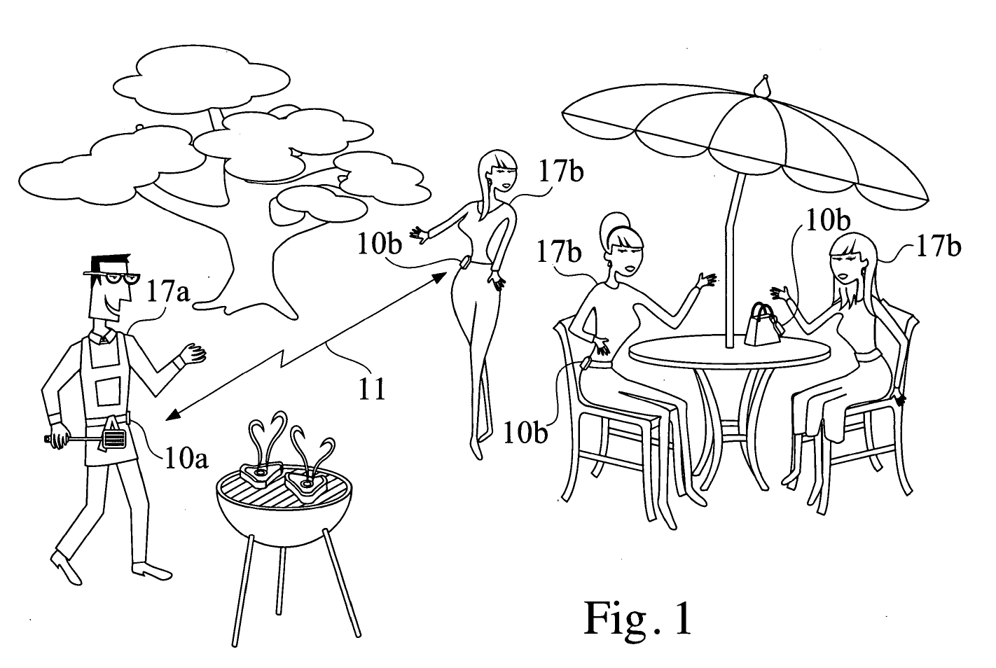

FIG. 1 is a representation of a private party. One of the hopes of some of the men and women who attend the party is that of finding a friend or a mate. In accordance with one embodiment of the present invention, a man 17a is shown wearing a MateFinder™ 10a clipped to his belt. A group of women 17b standing around or sitting at a table also have MateFinders 10b, which are clipped to their belts or purses or are concealed in their clothing.

In general, the MateFinder™ 10 is an electronic device which uses a radio to help find someone or something which fits a predetermined description or some preselected criteria. The term “radio” is intended to encompass any device or system that communicates wirelessly.

The radio may comprise any communication means, signal conveying device, system or process for emanating and/or receiving data, messages, information, sensation, manifestation, pattern, perception, or other intelligence. The radio used by the present invention may comprise a transceiver. This transceiver may include a separate transmitter and a separate receiver, or may utilize a single circuit for both functions. The transceiver may operate only as a transmitter for a period of time, may operate only as a receiver for a period of time or may transmit and receive generally simultaneously.

In this Specification and in the Claims that follow, the MateFinders™ 10 are generally identified as 10a when used by a man or an unspecified “first user” 17a, and as 10b when used by a woman or unspecified “second user” 17b. The use of the reference characters ending in “a” and “b”, which are also recited in the s as the “first” and “second” transceivers, 10a and 10b, are intended to assist the reader in understanding the invention, but do not connote any substantive differences in the device 10.

The man's MateFinder™ 10a continuously emits an automatic and generally continuous radio seeking or interrogation signal 11. This signal 11 illuminates or interrogates other MateFinder radios that are within range. The man 17a has programmed his MateFinder 10a with a set of attributes that describes himself, and this information is conveyed by the signal 11 emitted by his MateFinder. This signal 11 may also convey a description of the woman that he is interested in finding.

If the man's MateFinder signal finds a woman who fits his preselected set of criteria, his MateFinder issues a visual and/or audible alert. If the man's MateFinder signal contains a description that matches the woman's predetermined description of a suitable man, the woman's MateFinder alerts her to his presence. The location and/or identity of each person carrying the MateFinder is not initially available to the users. For example, in one possible use, exemplars of the device would be worn (possibly concealed) by a number of users attending a large private party or public function. Each user's MateFinder would emit its own interrogation signal, for example, first interrogation signal 11a, and second interrogation signal 11b. Users would be alerted to the presence of compatible types, along with an indication of the degree of correlation found and whether a selected matching signal's position is masked. The user would then have the option of unmasking his or her position to the emitter of a specific signal, possibly by changing the modulation scheme to one that allows its strength to be detected. Users could then approach each other by maximizing their indication of the other's signal strength. It should be possible to see the target person at a safe distance before making further contact. The present invention reduces the risk that is inherent in Internet or other forms of remote or electronic dating by allowing a user to evaluate a prospective match in person before initiating contact. Some of the embodiments may also enhance the user's privacy, because his or her personal data are not sent to a website or other third party.

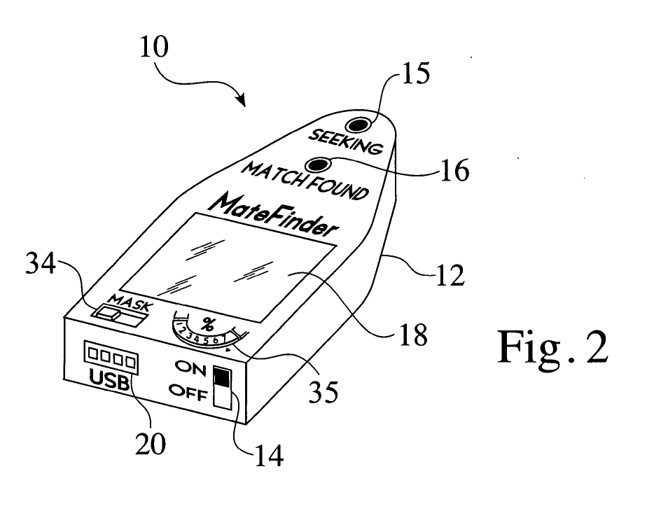

FIG. 2 furnishes a perspective view of one embodiment of the MateFinder device 10. A housing 12 made of plastic or some other suitable material encloses a radio (not shown in FIG. 2). A power switch 14 is located on the base of the housing 12. When the power switch 14 is turned to the “ON” position, an LED 15 labeled “SEEKING” flashes periodically to indicate that the MateFinder™ 10 is emitting a signal. When the MateFinder™ finds a match, an LED 16 labeled “MATCH FOUND” is illuminated. The illumination of the “MATCH FOUND” LED 16 may be accompanied by an audible alarm or tone, vibration or some other suitable means for alerting the user. In an embodiment in which two MateFinders 10 are used, each MateFinder includes a match indicator, 16a and 16b. A miniature LCD screen 18 is situated on one side of the MateFinder 10. This screen 18 is used to read messages which may be sent by the person who is detected by the seeking signal 11, or to view a list of possible matches detected by the MateFinder. A USB port 20 or some other suitable port for connecting the MateFinder 10 to a personal computer or some other appliance or device is located on the base of the device. In an alternative embodiment, the USB port 20 may be replaced or enhanced by a wireless connection. A “Mask” switch 34 enables the user to prevent another MateFinder™ from locating the user. A correlation switch 35 enables the user to adjust the level of matching that is performed by the MateFinder™.

For example, if the user turns the thumbwheel 35 toward the “10” indicator on the wheel, he or she is instructing the MateFinder™ 10 to seek out a stronger or higher level of correlation between the preselected qualities or attributes stored in his or her MateFinder 10 and a potential candidate. By turning the thumbwheel 35 down towards “1”, the MateFinder™ 10 reports matches that represent lower levels of correlation between preselected attributes and candidates.

In alternative implementations of the invention, the MateFinder™ 10 may be built into a bracelet, a necklace, a tie-clip, a hat, a shoe or some other suitable fashion item, article of clothing or ornament.

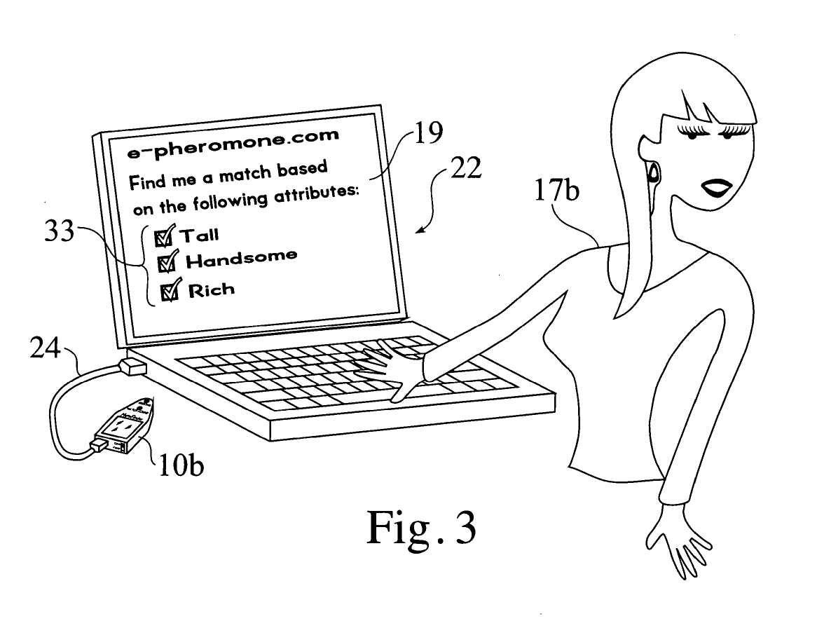

FIG. 3 depicts a woman 17b who has visited a website 19, www.e-pheromone.com. After connecting her MateFinder 10b to the USB port of her personal computer 22 with a cable 24, she is able to program her MateFinder™ 10b with two sets of attributes 33: a first set of attributes 33a that describes herself, and a second set of attributes 33b that describes her ideal mate. In general, an attribute is any form of data, criteria, information, measure of suitability, complementarity or compatibility; qualities or characteristics that describe a person, item, system, device or thing being sought by a user of the present invention. In one embodiment, two sets of attributes are employed, a first 33a, which pertain to the “seeking” user, and a second 33b, which pertain to the “target” user. Both of these sets of information may be entered into the personal computer 22 in response to prompts from the pages of the website 19. Software running on a server (not shown) which hosts the website then sends the data back to the woman's computer 22, where it is conveyed to the attached MateFinder™ 10 over the USB cable 24. In an alternative embodiment, the connection between the personal computer 22 and the MateFinder™ 10 may be wireless.

In one embodiment of the invention, the MateFinder™ 10 may be programmed using a personal computer 22 with an Internet connection and a website 19. Alternatively, the programming may be accomplished with just a personal computer 22 loaded with suitable software. In an advanced embodiment, the MateFinder™ 10 may be programmed without any other device or software by communicating directly with a website 19 over a wireless connection, or may be programmed using a keyboard or some other input means associated with the MateFinder™ 10.

In another embodiment, the website can also provide aliases for its members' e-mail addresses. In this way, e-mail is forwarded to their true address, which is kept secret. If a suitor is too persistent, a user can easily change her alias. The user may also block e-mails from unwanted suitors. The website may also provide a similar service for cellular telephone numbers, by furnishing a call-forwarding feature for a discreet call-in number.

The attributes 33 which may be selected by the user are virtually unlimited. In the case of a dating service, attributes 33 may be selected from an existing list of attributes 33. As an example, Table One presents attributes 33 which the website 19 displays may include:

TABLE ONE

| Category | Attribute |
|---|---|
| Gender | Male, Female |
| Age | |
| Appearance | Handsome/Knockout, |
| | Attractive/Cute |
| Marital history | Single, Divorced |
| Residence location | |
| Height | Tall, Average, Short |
| Weight | |
| Hair Color | Blonde, Brunette, Redhead |
| Occupation & Income | |
| Religiosity | Yes/No; Denomination |
| Political preferences | Conservative, Liberal, None |
| Interests or hobbies | |
| Educational level | |
| Social Class Marker | |

In this “electronic dating” embodiment, the user generally creates two sets of attributes 33: a first set 33a to describe herself or himself, and a second set 33b to describe his or her ideal match. In another embodiment, the user may only select one set, either only attributes that describe herself 33a, or only attributes that describe a mate 33b. A set of attributes 33 may include any number of qualities, numbering from one to a large number. Both sets of attributes 33 are stored in a non-volatile memory that is housed within the MateFinder 10. In one embodiment of the invention, one MateFinder 10a with a first memory 32a is employed to find a second MateFinder 10b with a second memory 32b. These preselected attributes may be revised by visiting the website 19, or may be generated using a software template provided with the MateFinder™ 10, which is then reprogrammed to incorporate the new data. In an alternative embodiment of the invention, the MateFinder™ 10 may be programmed directly using voice commands, or by using a keypad built into the device (not shown).

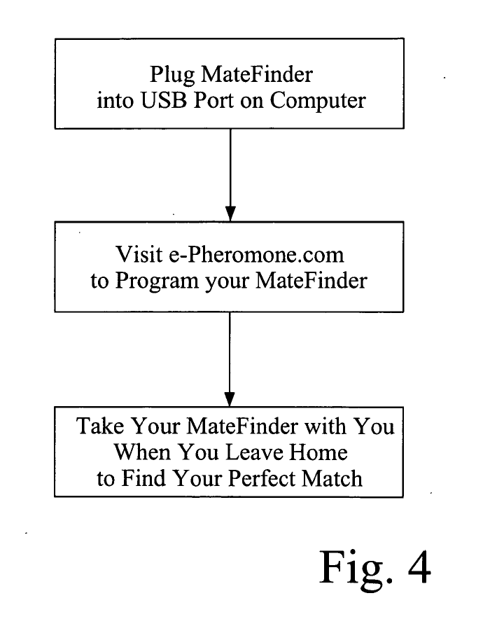

FIG. 4 offers a basic flowchart that describes how this embodiment of the invention is used. After acquiring a MateFinder™ 10, the user connects it to a personal computer 22. The user visits a website 19 to select two sets of attributes 33 that are stored in his or her device, or uses a software template supplied with the MateFinder™ 10. When he or she is ready to enter a social setting, or simply leaves home, he or she then turns the MateFinder™ 10 on, and takes it along.

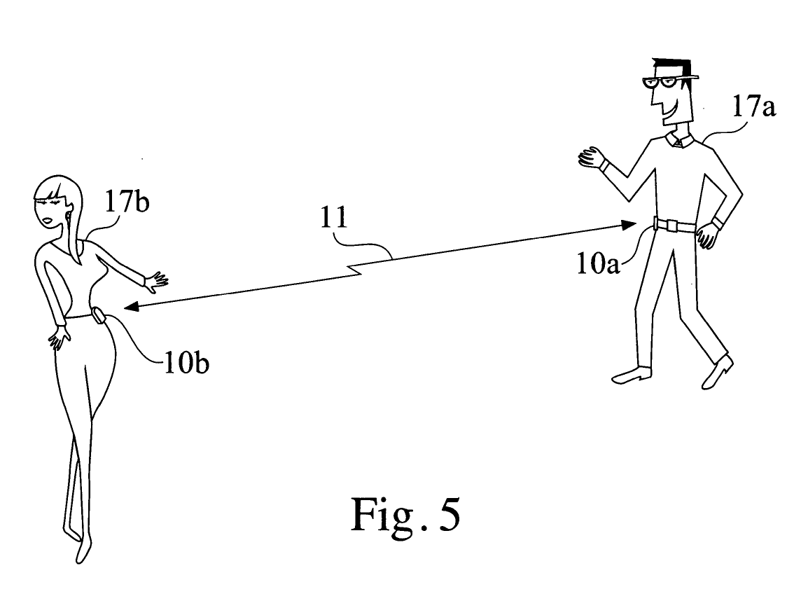

As shown in FIG. 5, the automatic and continuous seeking signal 11 finds a match. The woman 17b shown in FIG. 5, who may be attending a party, a concert or a sporting event, finds a match 17a based on her preselected attributes 33.

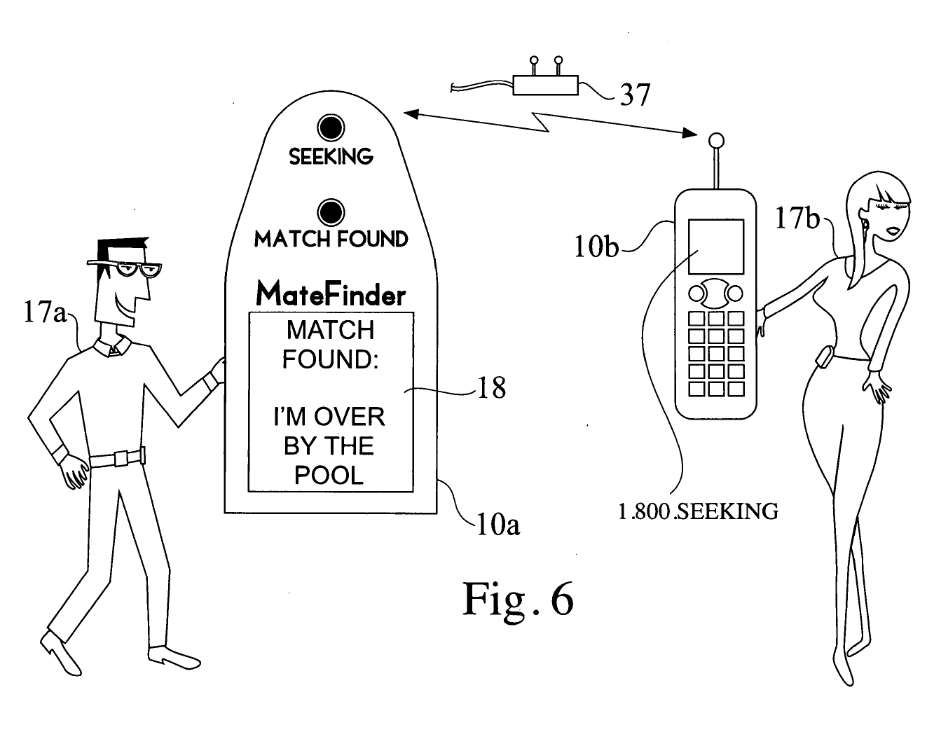

FIG. 6 reveals the operation of a more complex embodiment of the invention. After a man's MateFinder 10a has located a suitable match 17b, the man's MateFinder 10a conveys an address or some other identification message to the woman who has been matched. Her message may appear on the screen 18 of his device 10a. The woman 17b then has the option to communicate with the man 17a immediately. In one embodiment of the invention, the woman 17b may use her cellular phone to call a standard phone number, toll free number (such as 1.800. SEEKING) or a “900” number that generates revenue. In another embodiment, the MateFinder itself can provide text-messaging, either through its radiated signal or through a local network 37 or the Internet. The address or identification information which has been sent to her MateFinder 10b by the man's MateFinder 10a is displayed on her LCD screen 18. After dialing the toll-free number, she enters this address or identification information, and is then prompted to enter a text message, or to record a voice message.

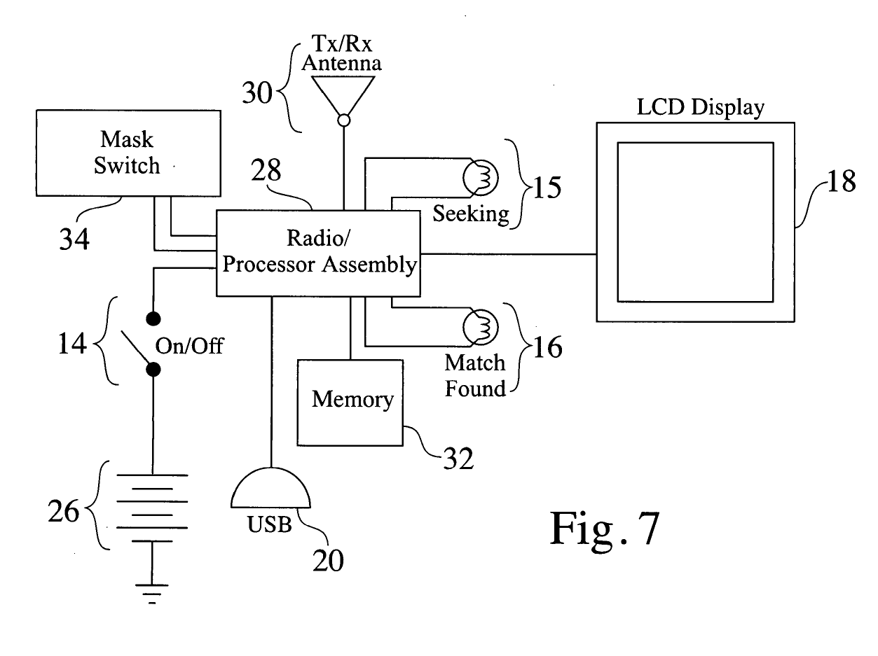

FIG. 7 is a simplified schematic diagram of the circuitry that may be employed to implement one embodiment of the present invention. The power switch 14 controls the flow of energy from a battery 26 that powers the MateFinder 10. When the MateFinder 10 is turned on, a radio/processor assembly 28 automatically and continuously emits a seeking signal 11 using antenna 30 over a short range. A USB port 20 is connected to the radio/processor assembly 28. The antenna 30 may be contained within the housing 12. When the radio/processor assembly 28 is broadcasting, the “SEEKING” LED 15 flashes periodically. When a match is found, the “MATCH FOUND” LED 16 illuminates, or some other audible or vibrating alarm is activated. The radio/processor assembly 28 is also connected to the LCD screen 18, which may be used to display short text messages that are received from another MateFinder 10.

The radio/processor assembly 28 is also connected to a memory 32, which is used to store attributes that describe the user and his or her ideal mate. The memory 32 may comprise any suitable non-volatile device, including, but not limited to a flash memory or hard-drive. In an alternative embodiment, a “MASK” switch 34, which is connected to microprocessor 36, may be included to allow the user to mask his or her location.

A suitable frequency for the radio emissions, such as one of the unlicensed “ISM” or “RF device” bands set aside by the United States Federal Communications Commission, is selected to avoid creating unwanted interference. The MateFinder 10 may be configured to emit and/or receive a variety of signals or emanations of energy. In the United States, some embodiments of the invention may use the 900 MHz, 2.1 GHz, 5.8 GHz, 59-64 GHz or some other radio frequency band. In other countries, other suitable frequency bands may be selected for the operation of the present invention. Other embodiments of the invention may employ light energy, voice commands, audible tones or ultrasonic emissions; mechanical, physical or chemical manifestations; radioactivity, or any other suitable means for communication.

In a more advanced embodiment of the invention, some or all of the discrete components described in FIG. 7 may be integrated on a single computer chip.

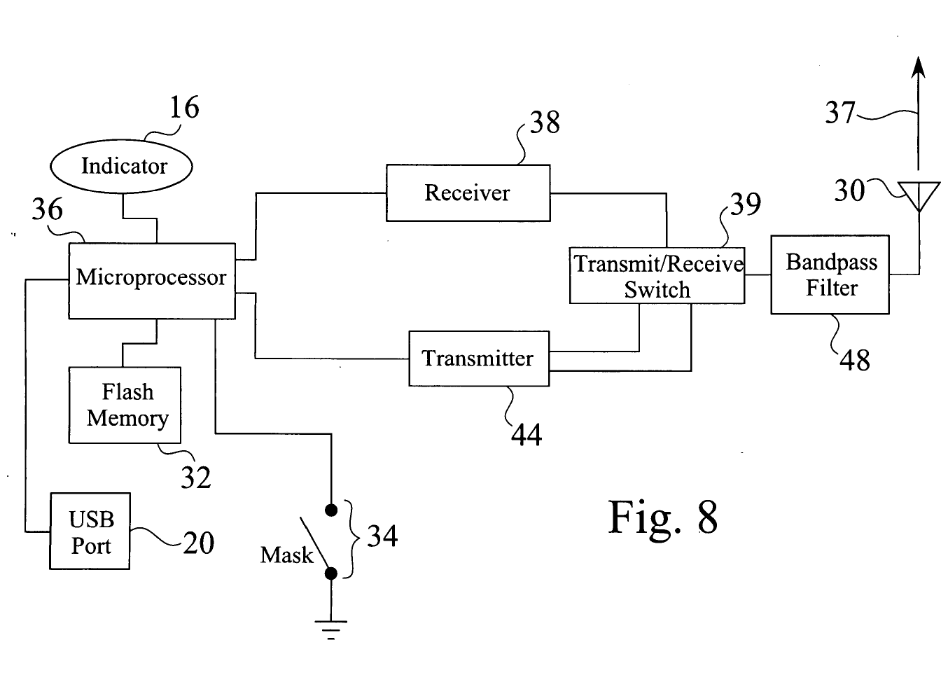

FIG. 8 provides a schematic diagram that illustrates one particular implementation of the invention. A microprocessor 36 is connected to a flash memory 32, a USB port 20 and an indicator 16. The microprocessor 36 is also connected to a receiver assembly 38 and a transmitter assembly 44. The outputs of the receiver 38 and the transmitter 44 are connected to an automatic transmit-receive switch 39, which, in turn, is connected to a bandpass filter 48 and an antenna 30.

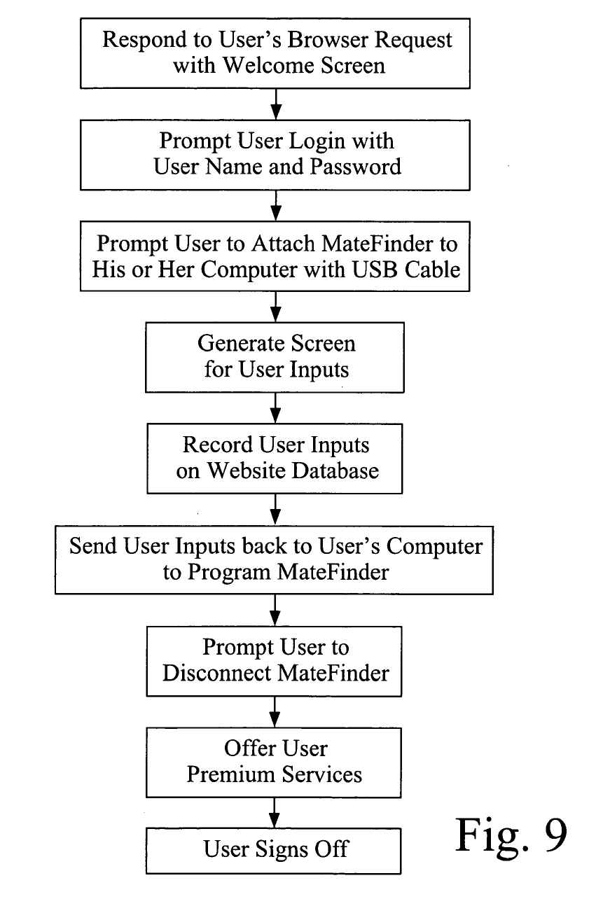

FIG. 9 offers a flowchart which depicts the basic operational steps of a particular software program that may reside at the website 19 used by the present invention. In the first step of the process, a user visits the website 19, such as e-pheromone.com. The user's browser requests information from the website 19, and the website responds by sending the user a welcome screen. The welcome screen invites the user to either create a new account, or to login to his or her existing account with a username and a password.

After the user has logged in for the first time, a new screen prompts the user to attach his or her MateFinder to his or her computer with a USB cable. After the user's computer has reported back to the website that the MateFinder is connected, the website generates a new screen that prompts the user to program his or her MateFinder using menu selections and/or a set of input fields.

After the user completes the selections, this information is recorded on a website database, and the website 19 sends the data back to the user's computer in a form that may be recorded in the MateFinder's memory. The user then disconnects the MateFinder, and may be offered a variety of premium services, such as background checks, certification of attributes or compatibility analysis, before he or she logs off.

In another alternative embodiment of the invention, the MateFinder may be designed to work in combination with an existing WiFi or similar wireless network 37 that is operating in the place where the user happens to be located. The user would be able to employ the wireless LAN or wired network (via a cable to the MateFinder), and would then be able to take advantage of all the connections offered by the Internet.

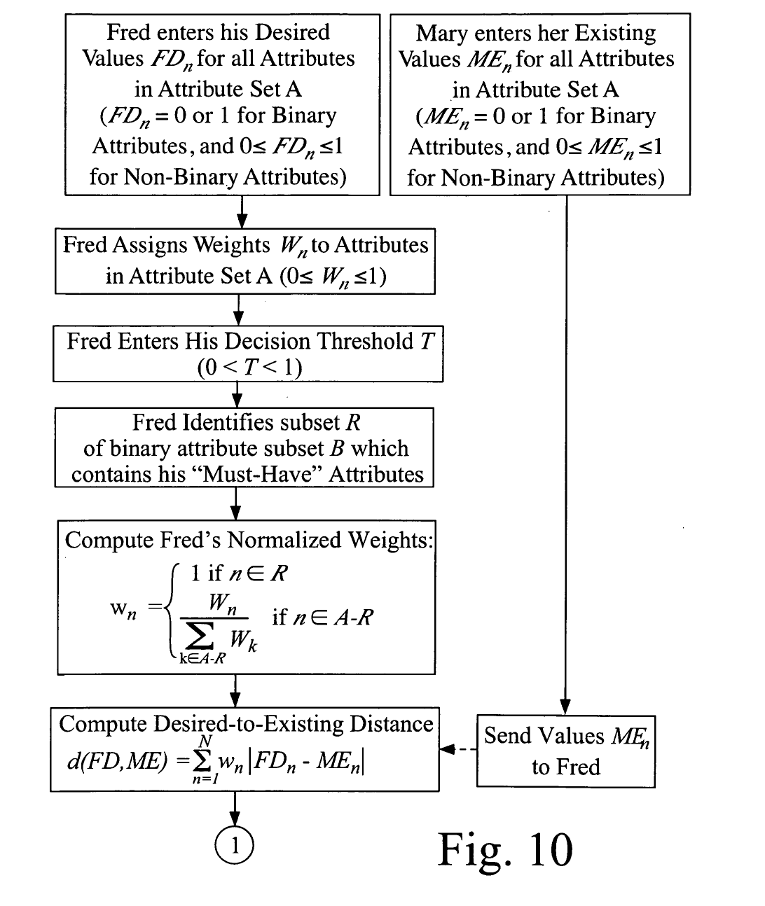

FIGS. 10, 11, 12 and 13 are flowcharts which depict Searching Methods Using Genetic Responsivity Measurements that may be used to compare and to correlate attributes of individuals, objects or systems to determine a good match.

The Searching Methods Using Genetic Responsivity Measurements that are described in this Specification may be embodied in software that is stored in the memory of one or more MateFinders, or may be incorporated in software that runs on a server that enables the operation of an Internet dating website. In general, the Searching Methods Using Genetic Responsivity Measurements provided by the present invention may be used by a wide variety of devices or processes or to compute a Genetic Responsivity Measurement, relationship or correlation between or among any individuals, objects or systems. The present invention provides a useful means for finding someone or something when the exact identity of the person or the object is not known, but desired attributes, characteristics or qualities of that person or object are known. In one specific implementation, the present invention may be used as an algorithm that enables a search engine to find a result based on a Genetic Responsivity Measurement Formula. When used in this Specification and in the Claims that follow, the term “Genetic Responsivity Measurement Formula” is intended to encompass any expression that enables the calculation of a Genetic Responsivity Measurement or correlation between or among any two individuals, attributes, objects, systems or entities.

### Genetic Responsivity Measurement as a Measure

In one embodiment of the invention, a matching algorithm that may be stored in the memory of a MateFinder is based on Genetic Responsivity Measurement. This measurement involves comparison of attributes of individuals which can be genetically based at the chromosomal level, or more generally can be outward manifestations of genetic characteristics (such as “red hair,” “blue eyes,” “height,” “skin color,” etc.)

In some cases, a high degree of compatibility of two individuals or entities occurs when they possess similar genetic characteristics. In these cases, the Genetic Responsivity Measurement will typically have a small value, indicating a small disparity in specified genetic attributes. However, research has shown that there are important cases where a high degree of compatibility occurs when the individuals or entities have differing but complementary genetic characteristics. In these cases, the Genetic Responsivity Measurement will typically have a large value, indicating a large disparity in specified genetic attributes.

Even though Genetic Responsivity Measurement is primarily intended to be based on genetic factors, the measurement can also be used to evaluate the disparity or similarity of non-genetic attributes, such as “a smoker,” “prefers classical music,” “enjoys travel,” etc.

As an example of a genetic responsivity measurement, suppose that there are two genetic attributes of interest, which have been assigned the numerical values x₁ and x₂ in one individual, and y₁, y₂ in another individual. These values can be represented as points (x₁, y₁) for the first individual and (x₂, y₂) for the second individual (it is convenient to visualize these points as lying in the plane). The formula for the Genetic Responsivity Measurement d between these two points is:

d=\sqrt{(x₁−x₂)²+(y₁−y₂)²} Expression 1

The x coordinate of a point can be regarded as a measure of one attribute of the point, which geometrically we might call the horizontal attribute. Similarly, the y coordinate is a measure of the vertical attribute. The Genetic Responsivity Measurement d is a joint measure of how closely the attributes of the two points match, with smaller values indicating a closer match. If d=0, the match is the closest that is possible to achieve, because this happens if and only if the two points are exactly at the same place.

The two points can be labeled with any identification or name. For example, the point (x₁, y₁) might be “Fred” and the point (x₂, y₂) might be “Mary.” If Fred and Mary are closely matched in both attributes, the Genetic Responsivity Measurement d between them will be small.

The Genetic Responsivity Measurement formula can be generalized to permit any number of attributes to be considered. It is convenient to use a different symbolism for the attributes and their values. Suppose there are N attributes. Let F₁, F₂, . . . , F_{N} be Fred's values for attributes numbered 1, 2, . . . , N. Similarly, let M₁, M₂, . . . , M_{N} be Mary's values for those same attributes. The Genetic Responsivity Measurement between Fred's and Mary's attribute constellations can be computed as

$$d ( F , M ) = \sqrt{{\sum }_{n = 1}^{N} {( {F}_{n} - {M}_{n} )}^{2}} \qquad Expression 2$$

Expression 2 has some possible shortcomings. First, the squaring operation puts more weight on attributes where the difference between Fred and Mary is large and less weight where the difference is small. Second, the range of values for each attribute has not been specified (a large value for a specific attribute might be 10, but on the other hand, it might be 100). Third, if some attributes should be weighted more heavily than others, there is no provision for performing this step.

All three shortcomings can be eliminated by changing the definition of Genetic Responsivity Measurement. The new definition is a variation of a measure used by mathematicians in “taxicab geometry.” Taxicab geometry is used in a city with only north-south and east-west streets, where at any given moment a taxi can only be moving in one of the four directions and cannot travel the shortest possible route to its destination as the crow flies, given by Expression 1. The new definition is:

$$d ( F , M ) = {\sum }_{n = 1}^{N} {w}_{n} \vert {F}_{n} - {M}_{n} \vert \qquad Expression 3$$

where ∥ denotes absolute value and wₙ is a number between 0 and 1 which assigns a weight to the nth attribute. The N weights have the property

$${\sum }_{n = 1}^{N} {w}_{n} = 1 \qquad Expression 4$$

which makes them relative weights. Also, the measure of each attribute Fₙ or Mₙ is also restricted to be a number between 0 and 1. Thus, the largest or most significant value of an attribute is 1, and the least smallest or least significant value, is 0.

With this definition, it is not hard to see that the Genetic Responsivity Measurement d(F, M) will always be a number between 0 and 1, regardless of the number N of attributes or the assignment of weight values (The weights must sum to 1, however. Later we'll describe an easy way for this to be done automatically). Fred and Mary's attributes are similar or not similar according to whether the Genetic Responsivity Measurement d(F, M) is close to 0 or close to 1. Of course, the thresholds for similarity must somehow be determined.

### Applications of the Genetic Responsivity Measurement Formula

The Genetic Responsivity Measurement formula given by Expression 3 can be used in a number of different ways:

### Example 1

### Matching What is Desired to What Exists

Suppose Fred is looking for a mate based on desired genetic attributes. These attributes can be at the chromosomal level, in which case they are likely to have been selected based on genetic research, or they can be outward manifestations of genetic characteristics. Each of the N attributes is assigned a value desired in his mate and also is assigned a weight Wₙ between 0 and 1 (note that W is capitalized). A 1 means the attribute is very important, and 0 means it is not at all important. Fred does not have to worry about the sum of the weights being 1, because the relative weights actually used will sum to 1. They will be computed by means of the formula

$${w}_{n} = \frac{{W}_{n}}{{\sum }_{k = 1}^{N} {W}_{k}} \qquad Expression 5$$

Meanwhile, Mary, being a potential mate, also has specific values for the same N attributes which characterize her genetically. However, she doesn't concern herself with weights, because they are important only to Fred.

The Genetic Responsivity Measurement d from Fred's desires to Mary's attributes is computed by means of the formula given by Expression 3, permitting Fred to decide if Mary is a good match for him. To clarify how the formula is being used, we use the notation FDₙ for the Fred's desired value of the nth attribute (if he doesn't really care about that attribute, he can assign the weight Wₙ=0 to it), and we denote by MEₙ Mary's existing value for that same attribute. Thus, the Genetic Responsivity Measurement Formula becomes

$$d ( FD , ME ) = {\sum }_{n = 1}^{N} {w}_{n} \vert {FD}_{n} - {ME}_{n} \vert \qquad Expression 6$$

where d(FD, ME) denotes the Genetic Responsivity Measurement from Fred's desired attribute constellation to Mary's existing constellation. In this case, the highest degree of compatibility from Fred's point of view occurs when the value of d(FD, ME) is small.

### Example 2

### Bidirectional Matching of Desires to What Exists

The above process can be reversed, with Mary matching her desired attribute values with Fred's existing values. This results in the Genetic Responsivity Measurement d(MD, FE). It would seem to be a good omen if d (FD, ME) and d(MD, FE) were both small. A simple joint measure of a good match in both directions is obtained by using the formula

$${d}_{BDE} ( F , M ) = \frac{d ( FD , ME ) + d ( MD , FE )}{2} \qquad Expression 7$$

which is again a number between 0 and 1. The subscript letters in d_{BDE} mean bi-directional, desired-to-existing.

### Example 3

### Matching What Exists to What Exists

In this alternative embodiment, the weights might be predetermined by a geneticist and/or psychologist whose education and experience provide a good basis for the weight values. Fred and Mary could each determine the values for their own attributes, or that might be better left in the hands of the geneticist and/or psychologist by means of genetic testing, an interview, and/or a questionnaire. The Genetic Responsivity Measurement between Fred and Mary would simply be determined by Expression 3.

### Example 4

### Handling “Must-Haves” and “Deal-Killers”

In matching what is desired to what exists (Example No. 1), Fred might want a good match to first be obtained using a specified subset R of the N attributes and no others. This is seamlessly accomplished as follows: Let the full set of attributes be called Set A. The indices of A are the positive integers from 1 to N, where N is the total number of attributes under consideration. Some of these attributes will only have the two allowable values 0 or 1, and we'll call them binary attributes. An example of a binary attribute at the chromosomal level might be “Has allele X at chromosomal locus Y” (1 for yes, 0 for no), and at the outward genetic manifestation level it might be “Has blue eyes” (1 for yes, 0 for no). Let B denote the subset of binary attributes. This subset contains some, but not all, of the indices in A.

Fred's first step is to choose desired values FDₙ for all attributes (Set A). Of course, he is only allowed to choose 0 or 1 for the binary attributes, but for the others he may choose any number between 0 and 1. He then chooses a weight Wₙ for each attribute, and a decision threshold T between 0 and 1 for the matching process. Then Fred identifies which attributes are “must-have” attributes, which must constitute a Subset R of the Set B of binary attributes. The relative weights wₙ for all attributes are then computed as follows:

$${w}_{n} = \{ \begin{matrix}1 if n ∈ R \\ \frac{{W}_{n}}{{\sum }_{k ∈ A - R}^{ } {W}_{k}} if n ∈ A - R\end{matrix} \qquad Expression 8$$

where the symbol ε means “is contained in,” and A−R denotes the set of attributes that are in A but not in R. When the weights wₙ determined by Expression 8 are used to compute the Genetic Responsivity Measurement d(FD, ME) in Expression 6, the Genetic Responsivity Measurement will be a number equal to or greater than 1 if the “must-have” attributes are not perfectly matched. In this case, Fred can decide not to proceed further. If the Genetic Responsivity Measurement is less than 1, it can be compared with Fred's threshold T. If d(FD, ME)<T a good match would be indicated.

### Example 5

### Changing the Attribute Scales

It might be desirable to have a different range of values for some or all of the attributes. The conversion is easily done. If the desired range of values for attribute number n is from a to b and the entered value x lies between these values, then the number y entered into the Genetic Responsivity Measurement formulas would be

$$y = \frac{x - a}{b - a} \qquad Expression 9$$

which is a number between 0 and 1.

### Example 6

### Converting from Genetic Responsivity Measurement to “Correlation”

The formula

c(F,M)=1−d(F,M) Expression 10

can be used to convert the measure of a match from a Genetic Responsivity Measurement to a “correlation,” which has the value 1 for a perfect match and 0 for the worst possible match.

### Example 7

### Detailed Example of Genetic Responsivity Measurement at the Chromosomal Level

For this application, the attributes can be the alleles found at specific genetic loci within the chromosomes of the individuals. An allele is one of a number of possible forms of a specified gene at a particular location or “locus” on a chromosome, and the allele generally varies from individual to individual. In one genetic application of Expression 3, the index n identifies a genetic locus within a specific chromosome, where the chromosome and the locus are common to the two individuals. The symbols Fₙ and Mₙ are numbers which respectively identify Fred's and Mary's allele at the locus having index n. These numbers are assigned to the alleles in such a way that the quantity |Fₙ−Mₙ| is a measure of the Genetic Responsivity Measurement between the two alleles, with a larger value indicating a greater Genetic Responsivity Measurement. The weight wₙ in Expression 3 is a measure of the relative importance of the Genetic Responsivity Measurement in the alleles at the locus with index n, with a larger value of wₙ indicating more importance. In some applications of Expression 3, such as sexual compatibility, a larger Genetic Responsivity Measurement, that is, a larger value of d(F,M), indicates a better match, and in other applications the goal is reversed—the smaller the Genetic Responsivity Measurement between individuals, the better the match.

As an example, Expression 3 might be applied to a region of human chromosome No. 6, called the MHC region. It has been determined that alleles in certain sections of the MHC region are involved in sexual attraction due to odors that appear in skin secretions. The significant loci are called “HLA-A” with 429 alleles, “HLA-B” with 751 alleles, and “HLA-DRβ1” with 511 alleles. As one of many ways to apply Expression 3, these three loci could be respectively indexed with n=1, 2, and 3. For Fred, the 429 alleles that can appear in HLA-A (identified by n=1) are labeled with the numbers 1-429, so that the value of F₁ for Fred is one of the numbers from 1 to 429, depending on which allele he has in the HLA-A locus. Similarly, for Mary, the value of M₁ can be one of the numbers from 1 to 429, depending on which allele she has in the HLA-A locus. The values of F₂ and M₂ for the 751 alleles in the HLA-B locus (n=2), and of F₃ and M₃ for the 511 alleles in the HLA-DRβ1 locus (n=3) are similarly assigned. For purposes of illustration, assume that F₁=32, M₁=321, F₂=522, M₂=324, F₃=99, and M₃=201. Assuming that the increasing order of importance of the three loci is HLA-B, HLA-DRβ1, and HLA-A, the weights in Expression 3 might be experimentally determined to be w₂=0.2, w₃=0.3, and w₁=0.5. Substituting all values into Expression 3 results in

$$\begin{matrix}d ( F , M ) = {\sum }_{n = 1}^{3} {w}_{n} \vert {F}_{n} - {M}_{n} \vert \\ = 0.5 \vert 32 - 321 \vert + 0.2 \vert 522 - 324 \vert + 0.3 \vert 99 - 201 \vert \\ = 144.5 + 39.6 + 30.6 \\ = 214.7\end{matrix}$$

as a measure of the Genetic Responsivity Measurement between Fred and Mary based on the alleles they have in the three loci.

### A Specific Implementation of the Invention

FIGS. 10, 11, 12 and 13 depict one particular implementation of the invention, and use the following terms, which are defined below. An “Actor” is a person, system or entity participating in the use of the Device, as defined below.

“Fred” is defined as the first-acting person or other entity. “Mary” is the second-acting entity. It is important to note that in this context, Fred and Mary may be of opposite or the same gender, and each may be of either gender, and either Fred or Mary or both may be non-living entities, objects, processes or systems.

“Attributes” in these drawings means tangible or intangible characteristics of the individual actors.

“Attribute Set A” is the set of all attributes used in the matching process. This set is the same for all actors. It is permissible for the actors to have different attribute sets. In this case, Attribute Set A is the intersection of the actors' attribute sets, that is, the set of attributes common to both actors.

“Value” is a description of the strength, degree, level, intensity, scope, scale, manifestation, propagation or perception of a particular Attribute. It may be a binary variable, such as gender, in which case it can only be “1” or “0,” (and is thus defined to be in Subset “B”), or it may be an analog variable, such as height, in which case it is selected from a scale which is known to all actors. For example, the actors might agree that a height Value of “1” should be assigned to heights of 7 feet or greater, while a height Value of “0” should be assigned to heights of 3 feet 6 inches or less, with Values for other heights linearly assigned to intermediate heights.

“Attribute Set B” is the subset of Attribute Set A consisting of all attributes which have binary values.

“Desired Attribute Value” is an Attribute which one actor wishes to find in an acceptable second actor.

“Existing Attribute Value” is an Attribute Value possessed by an actor.

“FDₙ” (Fred's Desired Value of Attribute n) is the nth member of a set of such Desired Attributes which has N members, and is given a Value as defined above. For example, Fred might prefer that Mary be about 6 feet tall, in which case he would assign a Value of 0.7 to his height preference, FDₙ, where a specific value of n identifies the height attribute.

“FEₙ” is Fred's Value for a particular Existing Attribute, such as height. If he is 6 feet, 2 inches tall, the Value of his height Attribute FEₙ might be 0.762, where a specific value of n identifies the height attribute.

“Weight” is the relative importance of each Desired Attribute FDₙ. For example, height might of minor importance to Fred, in which case he (or the psychologist in Drawing 12), might assign that Desired Attribute a Weight of 0.3; however, body mass index (BMI) might be of great importance to Fred, in which case he or the psychologist might assign a Weight of 0.9 to his BMI preference, FDₙ where a specific value of n identifies the BMI Attribute. Certain Attributes, such as gender, might be non-negotiable, in which case they would be assigned a weight of 1.

“Genetic Responsivity Measurement” is the overall weighted degree of match between Fred's Desired Attributes FD_{I}, FD_{N} and Mary's Existing Attributes ME₁, . . . , ME_{N}, or vice-versa, as calculated by Expression 6.

“Decision Threshold” is the maximum or minimum Genetic Responsivity Measurement at which an Actor or third party deems a match to be acceptable.

“Device” is a MateFinder, computer or other system. Each Actor may have a Device, or the Actors may share a Device, such as a server.

“Subset R” is a set of Desired Binary (Set B) Attributes, each of which, if not met, renders a match unattainable.

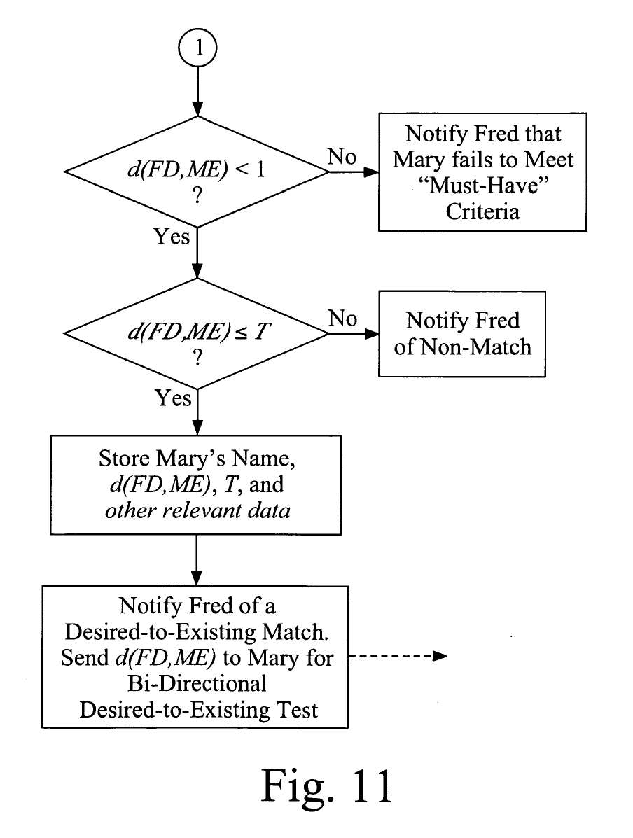

FIGS. 10 and 11 show the process by which Fred uses his Device to predict his compatibility with Mary, as follows:

1. Fred's Device has a list of A of N Attributes for which he must enter his desired Values. One by one, he enters a desired Value FDₙ for each of the Attributes in Set A into his Device.

2. Fred assigns a Weight Wₙ to each Attribute FDₙ.

3. Fred assigns a Value to his Decision Threshold T.

4. Fred selects a Subset R of binary Attribute Values in Subset B as defined above. Genetic Responsivity Measurements which are otherwise acceptable will be rendered unacceptable if these binary Attributes are not the same for both actors.

5. Fred's Device automatically computes a normalized weight wₙ for each of Fred's desired Attributes FDₙ, using Expression 8.

6. At the same time, Mary has the same list A of Attributes. She enters a Value MEₙ for each Attribute corresponding to the degree to which she possesses that Attribute into her Device. These are then Mary's Existing Attribute Values.

7. Mary's Device then transmits Mary's Values MEₙ to Fred's Device.

8. Fred's Device then computes the Genetic Responsivity Measurement between Fred's desired Attributes FD₁, . . . , FD_{N} and Mary's existing Attributes ME₁, . . . , ME_{N}, using Expression 6.

9. Fred's Device then compares Mary's existing Values of Attributes in Fred's Subset R to Fred's desired values of attributes in Fred's Subset R. If any of Mary's Attribute Values conflict with the value of an Attribute in Fred's Subset R, Fred's Device notifies him that Mary fails to meet his criteria (alternatively, Fred's device may remain silent).

10. Fred's Device compares the Genetic Responsivity Measurement between Mary's existing Attribute Values and Fred's desired Attribute Values. If the Genetic Responsivity Measurement is longer than the Decision Threshold T that Fred has established, it may notify Fred that a match does not exist, or remain silent. Fred's Device then enters an idle mode.

11. If Fred's Device determines that the Genetic Responsivity Measurement between Mary's existing Attribute Values and Fred's desired Attribute Values is less than or equal to Fred's Decision Threshold T it then stores such identification as may be provided by Mary's Device, along with the Genetic Responsivity Measurement, Threshold and other relevant data, and proceeds to Step 12.

12. Fred's Device then sends its calculated Genetic Responsivity Measurement to Mary's Device. Mary may then opt to have her Device calculate a Genetic Responsivity Measurement based on her desired Attribute Values MDₙ and Fred's Existing Attribute Values FEₙ, or take other action she deems appropriate.

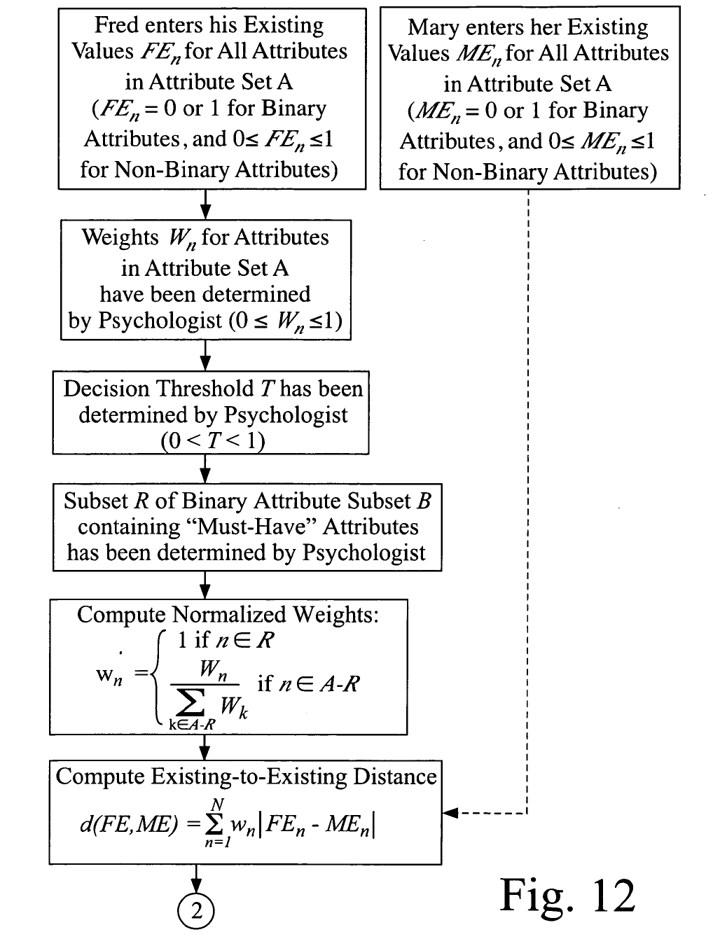

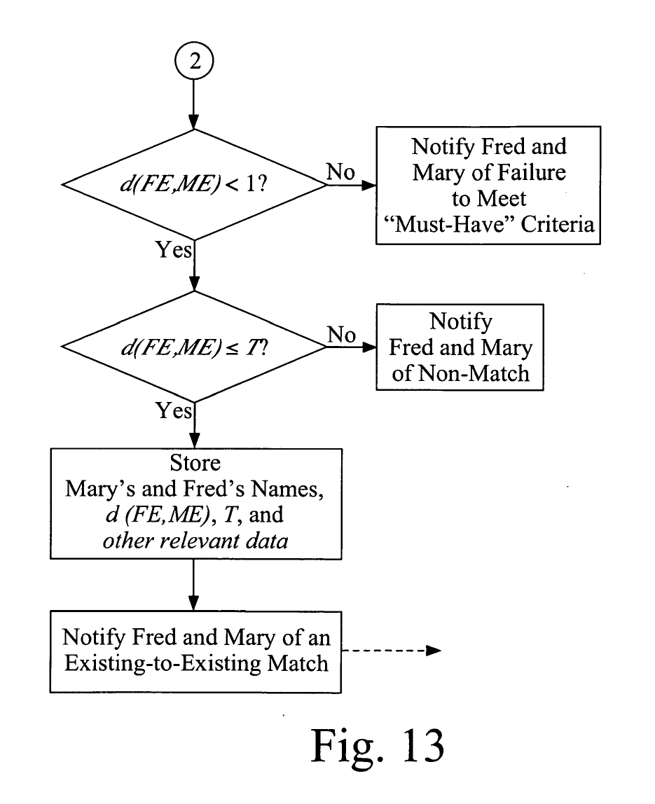

FIGS. 12 and 13 show the process by which Fred and Mary use the Device to compute their mutual compatibility, as follows:

1. Fred's and Mary's Devices have a list A of N Existing Attributes for which each must enter his or her Value. One by one, they enter a Value FEₙ and MEₙ for each of their Existing Attributes into their Devices or into a common system (hereafter Device in both cases).

2. A third party assigns a weight Wₙ to each Attribute n.

3. A third party assigns a Value to the Decision Threshold T.

4. A third party selects a Subset R of the binary Attributes in Subset B as defined above. Genetic Responsivity Measurements which are otherwise acceptable will be rendered unacceptable if the value of the binary Attributes in R are not the same for both actors.

5. The Device or Devices automatically compute a normalized weight wₙ for each of Fred's desired Attribute Values FDₙ, using Expression 8.

6. At the same time, Mary has the same list A of Attributes. She enters a Value MEₙ for each Attribute in Set A into her Device, which corresponds to the degree to which she possesses that Attribute.

7. Mary's Device then transmits Mary's Values MEₙ to Fred's Device.

8. Fred's Device then computes the Genetic Responsivity Measurement between Fred's Existing Attribute Values FE₁, . . . , FE_{N} and Mary's Existing Attribute Values ME₁, . . . , ME_{N}, using Equation 6, but with FD changed to FE and FDₙ changed to FEₙ.

9. Fred's Device then compares the values of Mary's Existing Attributes in Subset R to the existing Values of Attributes in to Fred's Subset R. If any of Mary's Attribute Values conflict with the value of an Attribute in Fred's Subset R, Fred's Device notifies Fred and notifies Mary's Device that they fail to meet matching criteria.

10. Fred's Device compares the Genetic Responsivity Measurement between Mary's existing Attribute Values and Fred's existing Attribute Values. If the Genetic Responsivity Measurement is greater than the Decision Threshold T, it notifies Fred and notifies Mary's Device that Fred and Mary fail to meet matching criteria.

11. If Fred's Device determines that the Genetic Responsivity Measurement between Mary's existing Attribute Values and Fred's existing Attribute Values is less than or equal to Fred's Decision Threshold T, it stores such identification as may be provided by Fred's and Mary's Devices, along with the Genetic Responsivity Measurement, Threshold and other relevant data.

12. Fred's and Mary's Device(s) then notify each of Fred and Mary of a satisfactory match.

### II. A Second Group of Embodiments:

### Using Searching Methods in an Internet Search Engine

In a second embodiment, the present invention is used to find information stored in a database, such as the Internet.

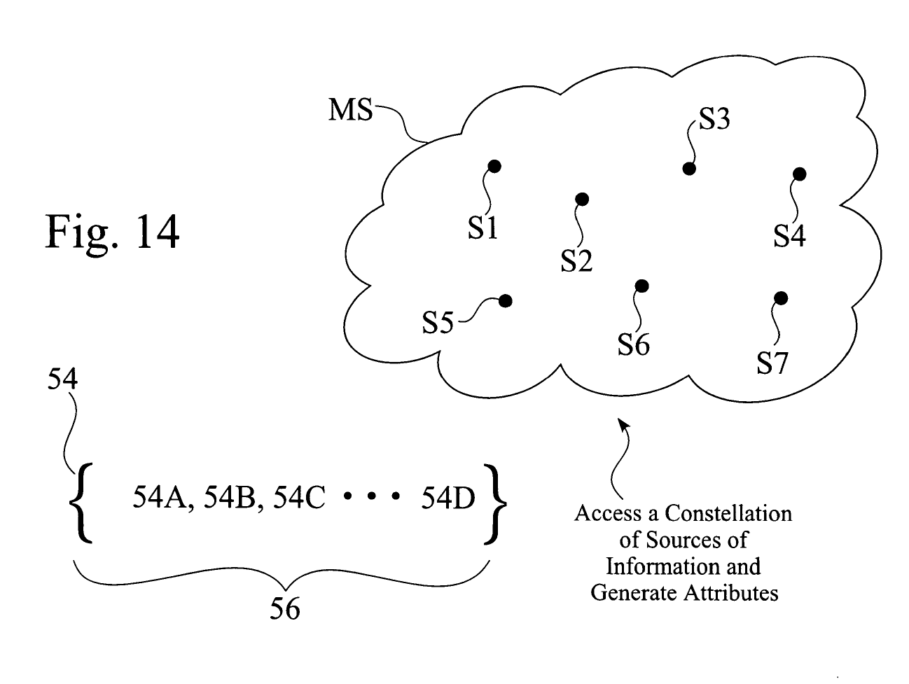

FIG. 14 is an abstract representation of a database or collection of information, such as the Internet. In this depiction, a number of webpages are characterized as sources of information (S1, S2, S3, S4, S5, S6, S7, . . . , SN), where N is the total number of webpages that may be accessed on the Internet. In a more general characterization, the entire set of webpages available on the Internet may be called the master set (MS) of sources of information.

When a search is conducted using a computer or some other electronic device to find a particular item of information that is available on the Internet, this search may be characterized as an attempt to find a target 56 within the master, set MS. The target 56 is assumed to be one of the many sources of information (S1, S2, S3, S4, S5, S6, S7, . . . , SN) that populate the master set MS.

In accordance with the present invention, a Difference Measurement formula search is performed by first selecting a set of attributes 54 which describe the target 56. In one embodiment of the invention, the formula for the Difference Measurement d between two points (x₁, y₁) and (x₂, y₂) in the plane is:

d=\sqrt{(x₁−x₂)²+(y₁−y₂)²} Expression 1

The individual attributes that are selected are represented as 54A, 54B, 54C, 54D and so on. As an example, suppose the target of the search is a Red Delicious apple. One set of attributes that may be selected by the searcher are listed in Table Two:

TABLE TWO

Fruit
Red
Sweet
Ripe in autumn

In general, when the set of attributes is relatively large, the search results converge or are focused more quickly, and the “quality” of the search is higher. In general, if the set of attributes contains only one or two entries, the search is not effective. When the number of entries in the set of attributes equals or exceeds four, five or six, the search narrows the field of sources of information to more effectively locate the target 56.

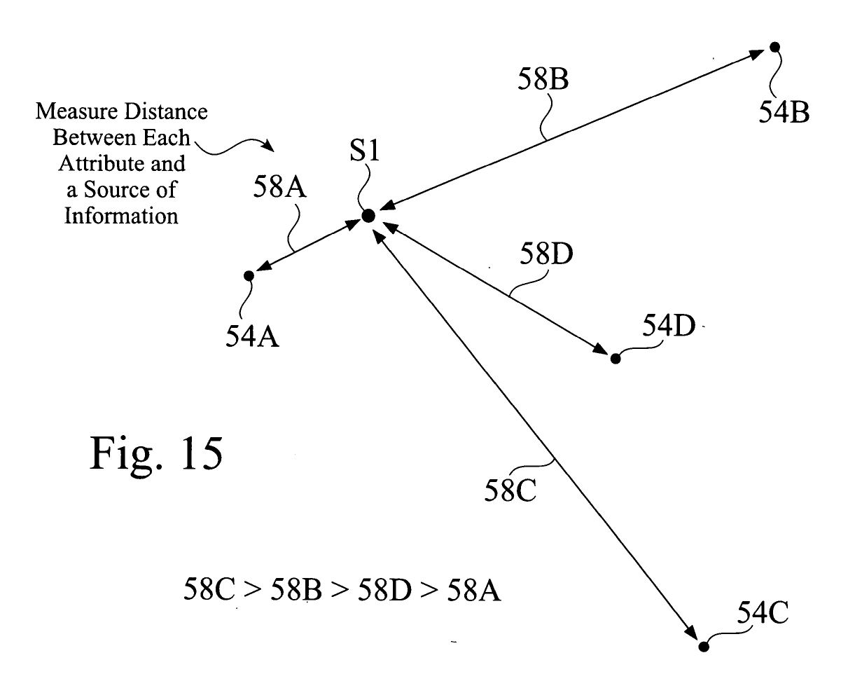

In accordance with the present invention, the sources of information in the master set MS and each of the selected attributes 54A, 54B, 54C and 54D may be graphically represented on a map, as shown in FIG. 15. In other words, from a mathematical point of view, all sources of information S and all conceivable attributes 54 occupy the same space. As a result, the Difference Measurements between each attribute 54 and each source of information S may be measured to generate a “relevance Distance Measurement.”

In one embodiment of the invention, a relevance Distance Measurement 58 is defined as a level of correspondence, similarity or “closeness of identity” between an attribute 54 and a source of information S.

FIG. 15 shows how four relevance Difference Measurements 58A, 58B, 58C and 58D are measured between pairs of attributes 54 and sources of information S. Difference Measurement 58A is the Difference Measurement measured between attribute 54A and source S1. Difference Measurement 58B is the Difference Measurement measured between attribute 54B and source S1. Difference Measurement 58C is the Difference Measurement measured between attribute 54C and source S1. Difference Measurement 58D is the Difference Measurement measured between attribute 54D and source S1.

In one embodiment of the invention, a Difference Measurement is computed by comparing an attribute 54 to a source of information S. Returning to Table Two, the attribute “fruit” is compared to sources of information (S1, S2, S3, S4, S5, S6, S7, . . . , SN). All of the sources of information that contain the term “fruit” are deemed to be relevant, those that do not contain the term “fruit” are deemed to be irrelevant. Any sources that contain the term “fruit” twice are deemed to be twice as relevant as those which contain the term only once. Any sources that contain the term “fruit” three times are deemed to be three times as relevant as those which contain the term only once, and so on. In this way, the relevance of all the sources of information may be arranged in a list that proceeds from high to low relevance.

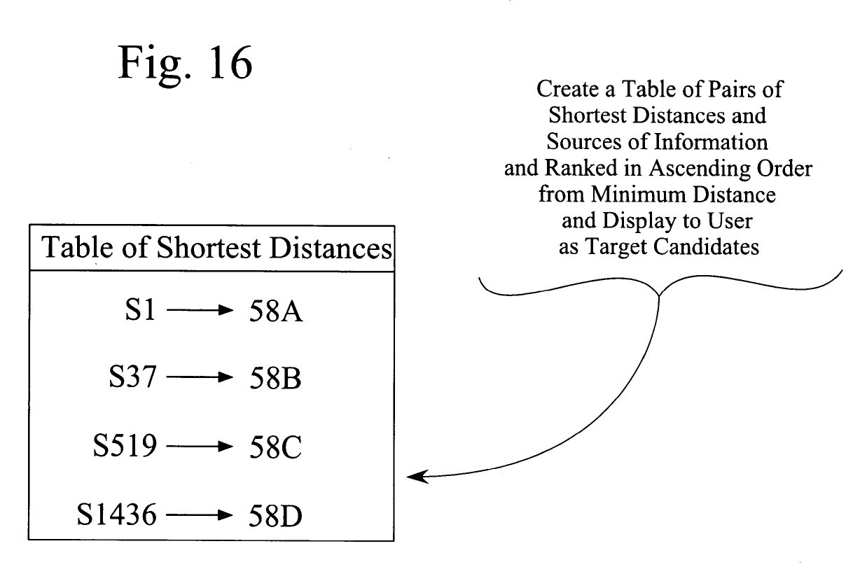

After each of the attributes 54 has been compared to the sources of information S, and the corresponding Difference Measurements 58 have been measured, a Table of Shortest Difference Measurements is created by selecting the shortest Difference Measurement that has been measured between one attribute and one source for each attribute. The shortest Difference Measurement signifies the highest relevance. FIG. 16 portrays this table, which includes four entries: one for each selected attribute 54A, 54B, 54C and 54D. The table indicates that four hypothetical sources, S1, S37, S519 and S1436, are the most relevant sources of information that correspond to the four selected attributes.

If the total number of sources of information S in the master set MS is a very large number, the present invention includes an additional preconditioning step which reduces the amount of Difference Measurement measuring that is required to complete a search. While the method depicted in FIGS. 14, 15 and 16 may be useful for a database of patient records in a physician's office, this method may be somewhat impractical when used as an Internet search engine.

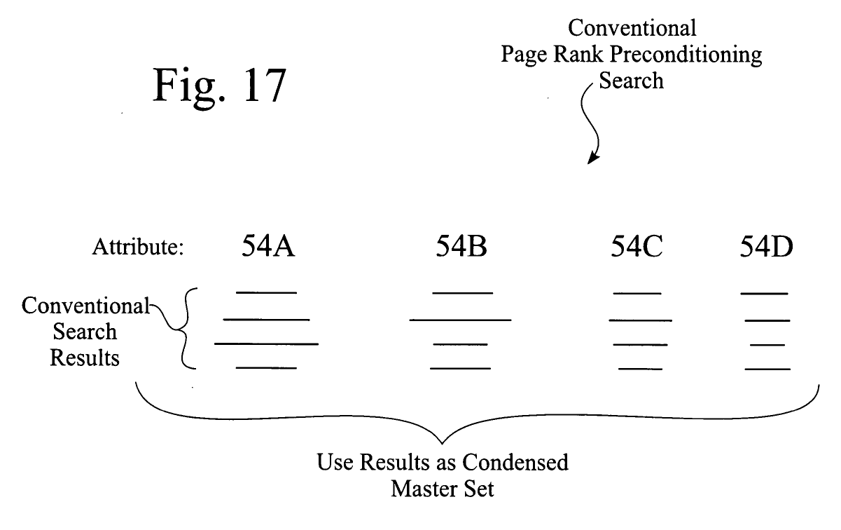

To improve the effectiveness of the method shown in FIGS. 14, 15 and 16, a conventional page rank search, such as the type of search that is offered by Google™, is first performed on each of the selected attributes 54, as shown in FIG. 17. The results of this set of preconditioning searches are then used as the master set MS. In virtually every instance, this results in a far smaller number of total sources that must be compared to attributes to create a set of shortest measured Difference Measurements.

In this Specification and in the Claims that follow, the term “conventional search engine” refers to existing tools that are commonly available today on the Internet, such as those employed by Google™, Yahoo!® and Ask™. The term “plurality of sources of information” includes, but is not limited to, entries in a document or a number of documents, spreadsheet or database; one or more web pages; a record or recording which contains some form of written information, data, audio, video, signal or some other form of intelligence or pattern; and/or the address or some other information or data concerning a radio frequency identification device (RFID).

### III. A Third Group of Embodiments:

More Advanced Searching Methods Using Difference Measurements with Additional User Inputs & Controls

In a third embodiment, additional controls and inputs are provided for the user to obtain better search results.

### A. Creating an Initial Search Attributes List

In accordance with a third embodiment of the invention, a user creates an initial search attributes list which focuses on the target of the search. The term “target” is used to describe the information, data, graphics, photo, rendering, video, audio, representation or other sensible expression of intelligence that the user seeks to discover or obtain as a consequence of the search. In this description of the third embodiment, and in the s that follow, the word “attributes” includes keywords, but has broader scope, which facilitates searching for items whose name or identity is unknown. When used in this Specification and in the Claims that follow, the term “keyword” generally refers to an entry for a conventional search engine which attempts to specify the exact name of the target of a search. The terms “searcher” and “user” are generally equivalent in this description, and may comprise a person or some automated combination of software and/or hardware.

As an example of the third embodiment, suppose the target of the search is a device which allows a person to button the collar of a dress shirt, even if the collar is otherwise too small. The searcher does not know the precise product name or part number which the manufacturer or seller uses to identify this device.

Instead of trying to find this device using a keyword, the searcher enters a list of attributes:

TABLE THREE

shirt
collar
button
makes collar larger

These attributes may comprise single words or phrases. The attributes may also include numbers, graphics, photos, drawings or other forms of specification.

The list of search attributes is entered into a conventional search engine, and then produces the information shown in the following matrix (boldface entries at the top and left side are not part of the matrix):

$$\begin{matrix} & {S}_{1} & {S}_{2} & {S}_{3} & {S}_{4} & … & & {S}_{N} \\ {A}_{1} & {f}_{11} & {f}_{12} & {f}_{13} & {f}_{14} & … & & {f}_{1 N} \\ {A}_{2} & {f}_{21} & {f}_{22} & {f}_{23} & {f}_{24} & … & & {f}_{2 N} \\ {A}_{3} & {f}_{31} & {f}_{32} & {f}_{33} & {f}_{34} & … & & {f}_{3 N} \\ ⋮ & ⋮ & ⋮ & ⋮ & ⋮ & & ⋮ & \\ {A}_{M} & {f}_{M 1} & {f}_{M 2} & {f}_{M 3} & {f}_{M4} & … & & {f}_{MN}\end{matrix} \qquad Expression 11$$

The boldface left column in Expression 11 is the initial search attribute list, and the boldface top row is a list of sources (i.e., “hits”) found by the conventional search engine that contain one or more attributes in the search attribute list. M is the number of attributes in the search attribute list, and is a reasonably small number (usually no more than 5 to 10, although it could be larger). N is the number of sources initially found by the search engine, and may be huge. For this reason, the matrix is not presented to the user. The entries fₘₙ in the matrix are the number of occurrences of the attribute Aₘ in the source Sₙ.

The search engine then ranks the sources according to relevance. There is an infinitude of ways this could be done. For the sake of concreteness, suppose that a relevance value Rₙ for each source is computed as follows:

$$\begin{matrix}{R}_{n} = relevance value for source {S}_{n} \\ = {W}_{n} {\sum }_{m = 1}^{M} {f}_{mn}\end{matrix} \qquad Expression 12$$

In this formula, Wₙ is a weight which is equal to the number of attributes in the search attribute list that appear at least once in source Sₙ. In other words, Wₙ is the number of nonzero terms in the summation appearing in Expression 12. The weighting by Wₙ is used because it seems that the larger the number of attributes appearing in a source, the more relevant that source is likely to be. If two sources each contain the same number of attributes (but not necessarily the same subset of attributes), they will receive the same weight, but could still have different values of relevance according to the size of the summation in Expression 12. The conventional search engine ranks the sources in accordance with the relevance values, with the sources having the larger values at the top of the list. Of course, it is possible (even likely) that some sources will have the same relevance values. When this happens, those sources could be sub-ranked within their relevance value according to some other criterion.

If it would be more satisfying to the user, the relevance values Rₙ could be converted into what might be called Difference Measurements Dₙ by the inversion shown in Expression 13:

$${D}_{n} = \frac{1}{{R}_{n}} \qquad Expression 13$$

or by some other monotonically decreasing function of Rₙ. In this case, sources having smaller Difference Measurements from the search attributes will be nearer the top of the source list.

### B. Interactive Search Refinement Using Linked Attributes

At this point, the user evaluates the initially found sources, starting at the top of the list, to determine if any meaningful search results have been found. In one implementation, the searcher utilizes linked attributes that the search engine locates in the sources which have been found. Linked attributes are defined as attributes within a source that are judged to be important by the search engine or the source, and are often unrelated to the user's search attributes. From each source that has been found, the search engine might select linked attributes based on thresholded frequency of occurrence in the source itself, on appearance within a list of attributes designed for the source, or on some other criterion. Of all the linked attributes that are discovered in all sources found, the search engine retains those which occur in more than a specified percentage P of the sources. Thus, a linked attribute list is created, which is presented to the user. Next to each linked attribute in this list is the number of sources in which it is found.

The linked attribute list is significant for two main reasons. First, it permits the user to quickly identify and eliminate a potentially large number of sources which are not relevant without having to examine a huge list of sources one source at a time. Secondly, it permits an expansion of the search to include sources that may be relevant, but which did not appear initially because the original attribute list created by the user was limiting the search too narrowly.

When the linked attribute list appears on the user's computer screen, the user can see at a glance attributes in addition to those he has chosen which can be used to sharpen his search, along with how many sources contain each linked attribute. With simple mouse clicks he can specify which of the linked attributes are non-relevant, so that the search engine can remove sources containing these attributes (hopefully a large number will be removed). Additionally, he can specify those linked attributes which he wants to add to his original search attribute list, allowing the search engine to find additional sources and do a re-ranking. When this is done, the additional linked attributes are added to his list of search attributes.

The above process of refining the search can be done as many times as desired. At all times during the search, two lists are being presented to the user—the current list of search attributes and an updated list of linked attributes.

### C. Method of the Third Embodiment

1. Initial Search: The user types in an initial search attribute list. This list is retained on the computer screen. The search engine extracts the information shown in the matrix (Expression 11) and finds N sources ranked according the relevance values or Difference Measurements computed by (Expression 12) or (Expression 13) (it is convenient to think of the sources across the top of the matrix (Expression 11) as having been ranked in order of relevance).
2. Optional Source Inspection: If desired, the user may inspect the sources near the top of the currently ranked list to see if anything useful has appeared.
3. Creation of a Linked Attribute List: From the sources found so far, the search engine creates and presents a linked attribute list on the computer screen, located next to the user's list of search attributes. Next to each attribute in the list is the number of sources in which that attribute is found.
4. Selection of Non-Relevant Linked Attributes: The user identifies non-relevant linked attributes and clicks on a “DELETE” box next to each non-relevant linked attribute. The search engine excludes sources containing these attributes and the information in the matrix (Expression 11) changes to include only that which is extracted from the remaining sources (again it is convenient to think of the sources across the top of the matrix (Expression 11) as having been ranked in order of relevance).
5. Selection of Additional Search Attributes: The user can add additional search attributes to the search attribute list in two ways: He can simply add any attribute that comes to mind, or he can specify items in the linked attribute list which he wants to add to the search attribute list by clicking on an “ADD” box next to item he wishes to include. The selected linked attributes are added to the search attribute list appearing on the computer screen. In response, the search engine alters the sources in its source list, thus changing the matrix (Expression 11), and re-ranks the entire source list.
At any time, the user can inspect the sources found thus far. A good time for inspection is when the number of currently retained sources is small enough to permit inspection of the entire source list.

### D. An Example of the Third Embodiment

The following example describes an actual experience with a conventional Google™ search attempt to find information about current research on “multipath mitigation in GPS (Global Positioning System) receivers.” “Multipath” refers to the propagation of a signal from a GPS satellite which arrives at a GPS receiver, not only via the direct path, but also after being reflected from nearby objects. An accurate measurement of the arrival time of the direct path signals from several satellites is needed for the receiver to establish an accurate position. However, the arrival of reflected signals can cause significant errors. It is similar to the problem of understanding what is being said by a speaker in a large hall, such as a gymnasium, when echoes are present.

The searcher first types in the keywords “multipath” and “GPS,” and the computer responds as follows:

TABLE FOUR
23,033 Sources Found:

| Current Search | Linked | |
|---|---|---|
| Attributes | Attributes | No. of Sources |
| Multipath | GNSS | 541 |
| GPS | movies | 13,540 |
| | plot | 10,220 |
| | radar | 43 |
| | mitigation | 455 |
| | interactive | 6,050 |
| | sonar | 15 |
| | entertainment | 16,128 |
| | television | 8,269 |
| | computer gaming | 14,979 |

Looking over the list of linked attributes, it seems surprising that the attribute “multipath” and/or “GPS” appears quite often in sources that are rich in the linked attributes “movies,” “plot,” “interactive,” “entertainment,” “television,” and “computer gaming.”

In addition to its use in GPS, “multipath” is also a term describing video entertainment in which the viewer can interactively alter the course of a plot. To further confuse the situation, the use of GPS receivers is a common element in the entertainment provided by television dramas. The search is simply swamped by the alternative meaning of “multipath.”

The user then decides to delete sources linked with the linked attributes “movies,” “plot,” “interactive,” “entertainment,” “television,” and “computer gaming.” Each deletion is indicated by a D in the right hand decision column in Table 5 below. The user also realizes that “GNSS” is an attribute that should be added to my list of search attributes, because this term stands for “Global Navigation Satellite Systems,” a broader term which includes the GPS system, but also others. All such systems have the same problems with multipath signal propagation. The searcher then adds the search term “mitigation” to focus more tightly on information about the mitigation of multipath. Each addition is indicated by an A in the right hand decision column. The searcher retains the sources having linked attributes “radar” and “sonar” because he knows that such systems also have multipath problems that may be of interest.

TABLE FIVE
23,033 Sources Found:

| Current Search | Linked | No. of | |
|---|---|---|---|
| Attributes | Attributes | Sources | Decision |
| Multipath | GNSS | 541 | A |
| GPS | movies | 13,540 | D |
| | plot | 10,220 | D |
| | radar | 43 | |
| | mitigation | 455 | A |
| | interactive | 6,050 | D |
| | sonar | 15 | |
| | entertainment | 16,128 | D |
| | television | 8,269 | D |
| | computer gaming | 14,979 | D |

The computer now responds as follows:

TABLE SIX
130 Sources Found:

| Current Search | Linked | No. of | |
|---|---|---|---|
| Attributes | Attributes | Sources | Decision |
| Multipath | radar | 35 | |
| GNSS | sonar | 13 | |
| GPS | spatial | 72 | D |
| Mitigation | antenna | 40 | D |
| | maximum likelihood | 32 | |
| | signal processing | 21 | A |

As seen in Table 6, the linked attributes “radar” and “sonar” still appear, and new linked attributes “spatial,” “antenna,” “maximum likelihood,” and “signal processing” have now appeared. These new items did not appear in the earlier linked attribute list because their frequency was significantly smaller than the attributes “movies,” “plot,” “interactive,” “entertainment,” “television,” and “computer gaming” which were previously deleted. However, after the sources containing these non-relevant attributes had been deleted, the new linked attributes occurred in a significantly larger percentage of the remaining sources, thus exceeding the percent threshold P. Consequently they now appear on the new linked attribute list.

As indicated above in the last column of Table 6, the searcher decides to retain the linked attributes “radar” and “sonar” for the reasons cited previously. However, he decides to delete the sources with linked attributes “spatial” and “antenna” as shown in Table 6 because these relate to multipath mitigation outside the receiver. The searcher is only interested in multipath mitigation techniques that use signal processing within the receiver. Therefore, as shown above in Table 6, he decides to add “signal processing” to the list of search attributes. Because the searcher knows that the linked attribute “maximum likelihood” is a mathematical method for multipath mitigation, this term is not added to the search attribute list in order to avoid too much limitation in the search. In addition, this term is not deleted, because doing so might remove some sources of interest.

The computer now responds with:

TABLE SEVEN
65 Sources Found:

| Current Search | Linked | No. of | |
|---|---|---|---|
| Attributes | Attributes | Sources | Decision |
| Multipath | radar | 35 | |
| GNSS | sonar | 13 | |
| GPS | maximum likelihood | 32 | |
| mitigation | correlation | 22 | |
| signal processing | | | |

At this point the searcher judges the number of found sources to be small enough for individual examination. Furthermore, the new linked attribute “correlation” is a good omen, because the searcher knows that it is strongly related to multipath mitigation by certain signal processing methods.

### E. Alternative Embodiments

Additional features which may be added to this third embodiment include, but are not limited to, the following:

1. The percentage threshold P for screening linked attributes can be made variable, even interactively changeable by the user as he proceeds through the search.
2. The user's computer may be configured to maintain a log of the entire search session, permitting the user to backtrack and change his inputs at any stage, or optionally to store the log as a file if he might want to reinstate the search at a later time.
3. Boolean logic such as “AND” and “OR” can be used in the search attribute list. For example, the searcher might have started the search with a single entry “GPS & multipath” in the search attribute list, which probably would have reduced the number of non-relevant sources citing multipath in connection with entertainment.
4. Case sensitivity or non-sensitivity may be incorporated into the search.
5. Other filters, such as numerical ranges for dates, sizes, weights or power may be used in conjunction with the search.

### F. Comparisons to Conventional Search Engines

The third embodiment of the invention offers the following advantages when compared to conventional search engines:

1. The user is provided with an efficient and interactive means of deleting, as well as adding, attributes used in the search without having to re-initiate the search.
2. At all stages of the search the user is kept informed of the effects of his attribute add/delete decisions by being able to view the linked attribute list and the number of sources associated with each linked attribute in the list.
3. At any time during a search the user can change the threshold P for the percentage of sources in which a linked attribute is found. The linked attribute is retained in the attribute list if and only if this threshold is exceeded.
4. A log of the entire search is continuously maintained, which includes a history of all attributes entered and deleted, and all results of the search at each stage. At any time the user can backtrack to any previous point in the search and modify his additions or deletions of attributes.

### G. A Fourth Embodiment

In a fourth embodiment, the matching algorithm comprises three steps:
encoding (mapping customer inputs to a vector space);
pairwise metric evaluation (computing metric between customers vector and
vectors of other customers in area); and
decoding (mapping of metric value to customer usable format).

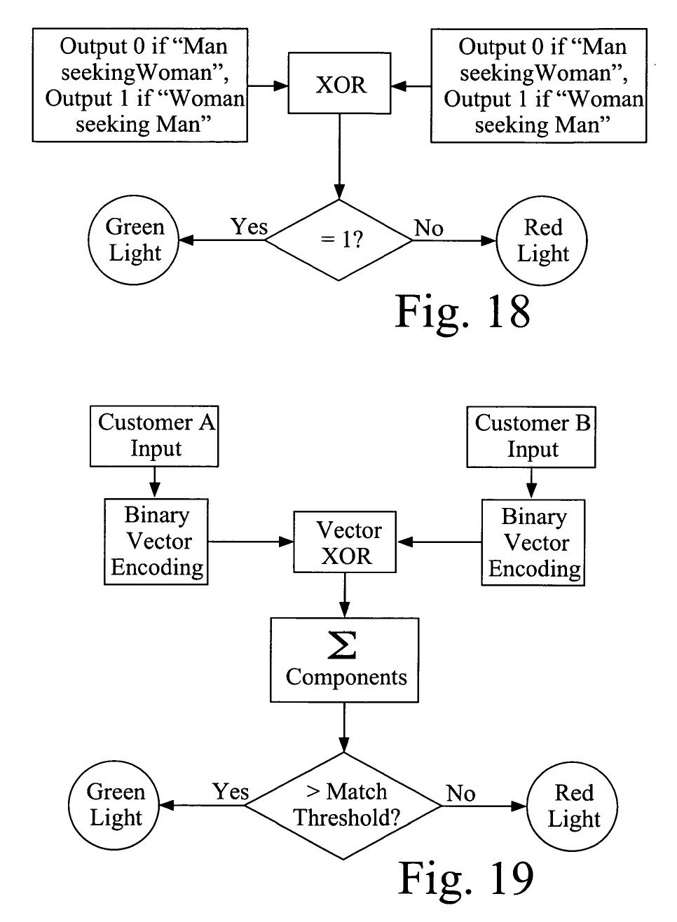

The encoding could be as simple as mapping “man seeking woman” to zero (0) and “woman seeking man” to one (1). Then the metric could be as simple as an exclusive or (XOR) operation. The truth table for XOR is 0 XOR 0=0, 0 XOR 1=1, 1 XOR 0=1, and 1 XOR 1=0. Then the decoding could be as simple as mapping 1 to a green light and mapping 0 to a red light, as shown in FIG. 18.

In the most general case, the vectors may comprise an ordered list of N values, and each value can be from a different set. For example, consider N=2 and the set for the first position is {0, 1, 2, 3, 4}, and the set for the second position is {Yes, No}. {Then 3, Yes} is a possible vector. In practice, we can restrict ourselves to an N-dimensional vector space over some field. We can encode any ordered list of N values into a vector in such a space with appropriate choice of underlying field. The encoded vector is preferable to an ordered list of values when computing metrics.

Possible fields include Z₂={0, 1}, Z_{P}={0, 1, 2, . . . , P−1} where P is a prime, Q (the rational numbers), R (the real numbers), and C (the complex numbers).

Another alternative, which is shown in FIG. 19, is binary encoding, Z₂, and sum of XOR as the pairwise metric to facilitate implementation in logic gates. The simplest pairwise vector metric would be the sum of component-wise XORs, assuming binary encoding:

$$Metric = {\sum }_{k = 0}^{N - 1} {a}_{k} ⊗ {b}_{k} \qquad Expression 14$$

where a=(a₀, a₁, . . . , a_{N-1}) is the vector for one customer and b=(b₀, b₁, . . . , b_{N-1}} is the vector for another customer.

### Other Possible Metrics Include:

a) sum of absolute values of component-wise differences:

$$Metric = {\sum }_{k = 0}^{N - 1} \vert {a}_{k} - {b}_{k} \vert \qquad Expression 15$$

b) sum of squares of component-wise differences:

$$Metric = {\sum }_{k = 0}^{N - 1} {( {a}_{k} - {b}_{k} )}^{2} \qquad Expression 16$$

c) sum of some other power (could be positive or negative, could be integer or real) of component-wise differences:

$$Metric = {\sum }_{k = 0}^{N - 1} {( {a}_{k} - {b}_{k} )}^{β} \qquad Expression 17$$

d) sum of component-wise products (dot product):

$$Metric = {\sum }_{k = 0}^{N - 1} {a}_{k} {b}_{k} \qquad Expression 18$$

e) sum of logarithms of absolute values of component-wise differences:

$$Metric = {\sum }_{k = 0}^{N - 1} log ( \vert {a}_{k} - {b}_{k} \vert ) \qquad Expression 19$$

f) sum of some other function of component-wise differences:

$$Metric = {\sum }_{k = 0}^{N - 1} ( {a}_{k} - {b}_{k} ) \qquad Expression 20$$

The above metrics can be weighted using a weight vector which is component-wise multiplied by each component-wise difference, or component-wise product, prior to the summation. For example with the component-wise products metric:

$$Metric = {\sum }_{k = 0}^{N - 1} {w}_{k} {a}_{k} {b}_{k} \qquad Expression 21$$

where w=(w₀, w₁, w_{N-1}) is the weight vector.

Another weighting method is to use an N×N weighting matrix, where N is the dimension of the encoded vector space. The weighting matrix is multiplied by the transpose of the customer's vector and the other customers' vectors are multiplied by the resulting vector to form a scalar:

Metric=a^{T}Wb Expression 22

where W is the weighting matrix and the boldfaced T denotes matrix transpose.

The decoding can be a map into anything customer friendly. For example, a “match score”, say from 1 to 10. Another example is “match/no match”, displayed with lights, colors, or the words themselves.

Matching can be made adaptive in two ways. In the first method, the encoded vector is adapted (changed) based on customer feedback on prior matches. In the second method, the weighting vector, or weighting matrix, is adapted (changed) based on customer feedback on prior matches. Both methods could be used simultaneously. The feedback could be binary—“I liked the match” or “I did not like the match”. Another possibility is a numerical value, say 1 to 10 with 10 being a great match and 1 being a very poor match. Still another possibility would be multi-characteristic feedback, such as a rating form.

### H. A Fifth Embodiment

### A Website for Finding Generic Products

In yet another implementation of the invention, the Applicants' Searching Methods Using Difference Measurements may be used to find a generic part or product when the searcher does not know the name of the part or the appropriate manufacturer's part number. As an example, suppose a person needs a replacement part for an appliance or automobile, but does not have a part number. The present invention may be used to find the correct replacement part by performing a search that employs a set of attributes that describe the needed part or product. The search may find a generic part which is priced far lower than the equivalent part that originates with the original manufacturer. This fourth embodiment may also be used to find generic drugs, medications or other medicinal preparations.

As a particular example, suppose a person wants to purchase a device which wirelessly transmits both the audio and video portions of a television signal from a first television in one room to a second television in another room in his house. This person is vaguely aware that such a device is available for sale, but does not know the manufacturer or the name that the manufacturer uses to identify this product.

If this person uses a conventional search engine such as Google™, he must rely upon keywords, such as: television, wireless, transmitter, and in-home. When used in a conventional search engine, this particular set of keywords does not produce results that are especially helpful, because none of these keywords match the name that the manufacturer uses to describe the sought after device.

If, on the other hand, the searcher were able to conduct a search using these same keywords as attributes using the Applicants' Searching Methods Using Difference Measurements, the present invention would identify several products that are currently available for sale and that are identified as “Video Senders.”

### V. A Fifth Group of Embodiments: Relationship Prediction System Opening an Account, Obtaining a Test Kit and Submitting Attributes to an Internet Dating Service

The present invention comprises methods and apparatus for predicting good relationships or matches. Merriam-Webster's Online Dictionary defines the word “relationship” as:

“1: the state of being related or interrelated, e.g., studied the relationship between the variables
2a: the relation connecting or binding participants in a relationship: as a kinship
2b: a specific instance or type of kinship
3a: a state of affairs existing between those having relations or dealings, e.g., had a good relationship with his family
3b: a romantic or passionate attachment”

In this Specification, and in the Claims that follow, the term “relationship” is used to connote a connection, association, affiliation or formal union between two persons. In particular, the relationships described and claimed in this patent application pertain to relationships which are premised, engendered or motivated by:
1. a correlation of self-describing attributes and the ideal-match attributes of another person; and
2. a first person's natural response to the genetic attributes of second person

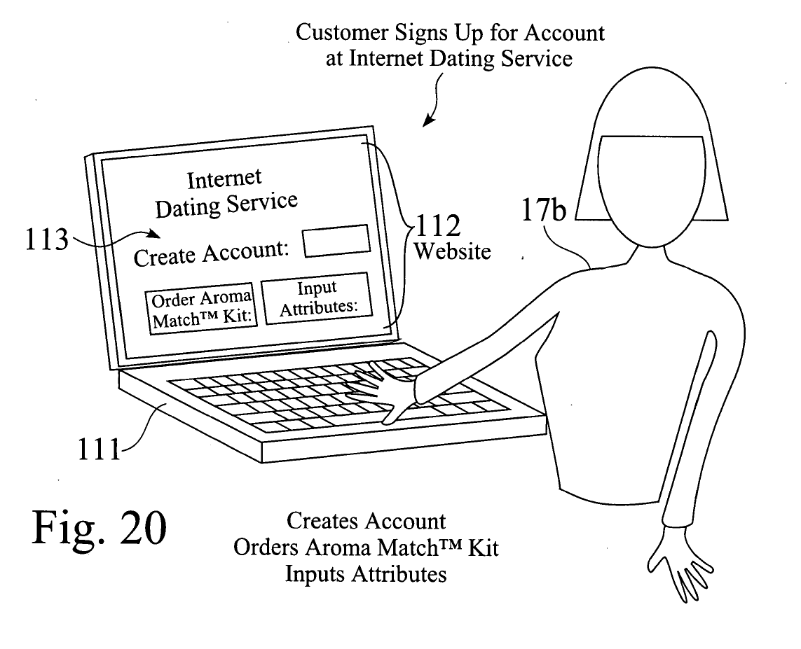

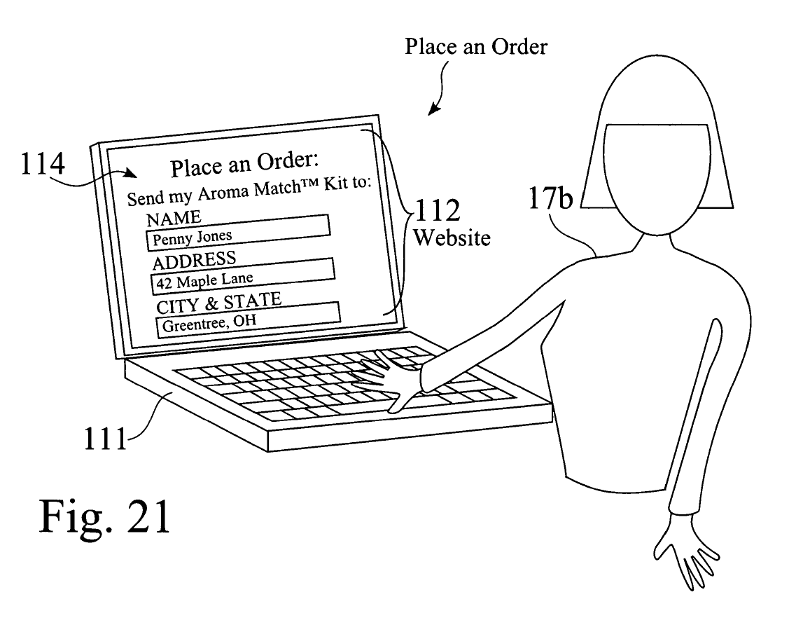

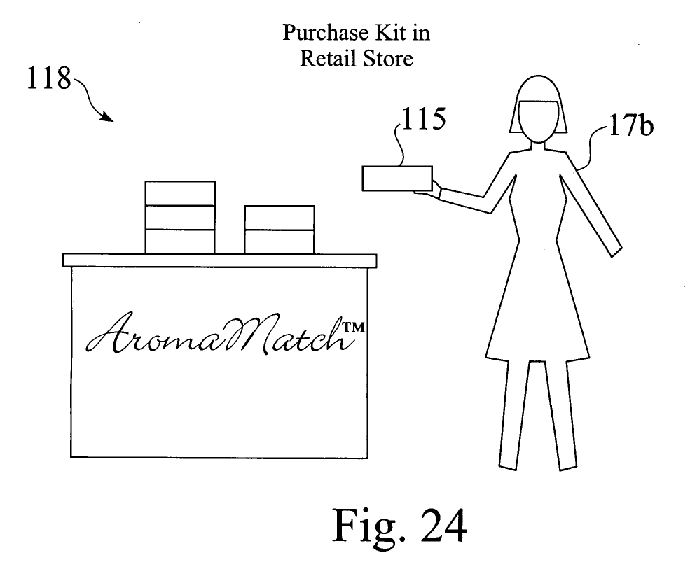

In one particular embodiment of the invention, the prediction of good relationships is predicated on a female's “responsivity.” FIG. 20 shows a woman 17b using a personal computer, personal digital assistant, web-enabled cellular telephone or any other similar information appliance 111 to visit an Internet Dating Service 112 website. The view in FIG. 20 shows a web page 113 for opening a new account. Once the new account is established, the woman 17b proceeds to another page 114 on this website as shown in FIG. 21, which enables the woman 17b to place an order for an AromaMatch™ Test Kit 115 (FIG. 24). “AromaMatch” is a Trade & Service Mark owned by the Assignee of the Present patent application. The website “www.aromamatch.com” is also owned by the Assignee of the Present patent application. In one embodiment, the Test Kit 115 will be delivered to the customer 17b by the U.S. Mail, or a courier such as UPS™ or Federal Express®.

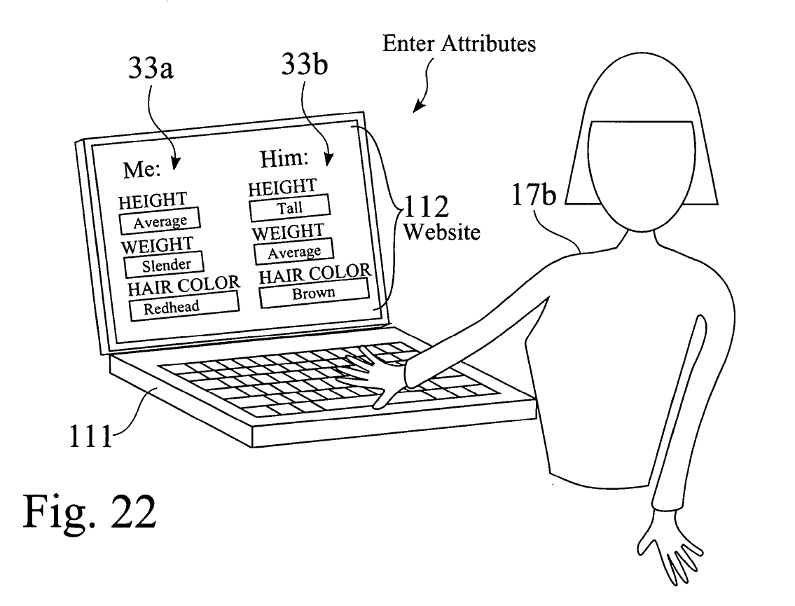

FIG. 22 illustrates the same woman 17b entering attributes which describe herself 33a, as well as attributes which describe her perception of a good match 33b. These attributes 33a & 33b may describe physical characteristics, personality traits, educational levels, jobs or careers, personal goals, hobbies, activities or any other information that may provide a basis for predicting a good match.

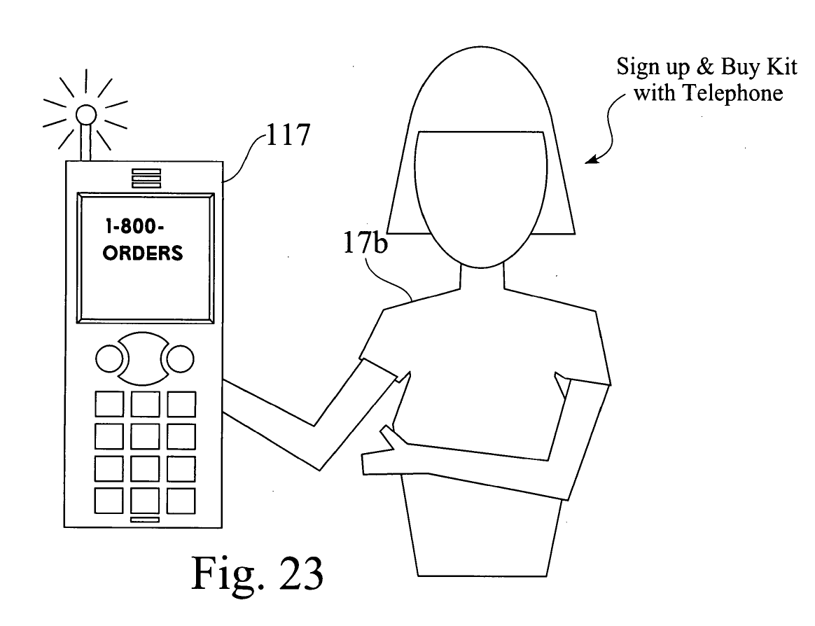

FIG. 23 depicts an alternative embodiment of the invention, in which the woman 17b uses a telephone 117 to open an account, place an order and/or submit attributes 33a and 33b to the Internet Dating Service over the phone.

FIG. 24 reveals another alternative embodiment, in which the woman 17b visits a retail store 118 to open an account, purchase a Test Kit 115, and/or fill out a questionnaire which furnishes attributes 33a and 33b to the Internet Dating Service 112, or other dating or introduction service.

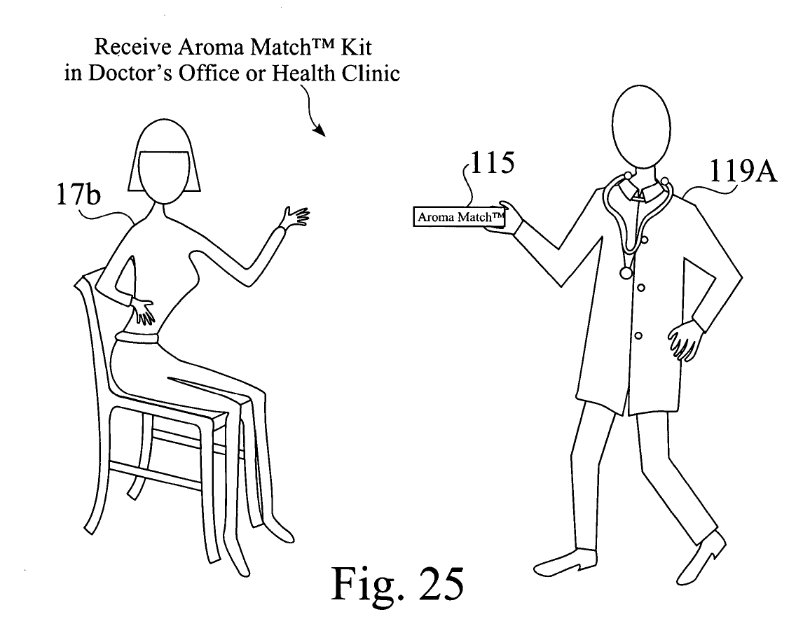

FIG. 25 supplies a view of yet another alternative embodiment of the invention, in which the customer 17b may open an account, purchase a Test Kit 115, and/or fill out a questionnaire to supply attributes 33a and 33b at a doctor's office or health clinic. The woman 17b may receive a Test Kit 115 from a physician, nurse, medical assistant or some other health care provider 119A. The customer 17b may provide her tissue sample while visiting the doctor's office, which is then certified by the doctor 119A before it is submitted to the laboratory. In this embodiment, the physician provides the Test Kit 115, and obtains the tissue sample. The physician 119A then sends the tissue sample to a laboratory for analysis, and also certifies that the sample is from a particular person. In this example, the physician acts as a “notary” who insures the identity of the source of the sample. This implementation of the invention guards against the fraudulent submission of a tissue sample from a person who might attempt to supply a misleading identity.

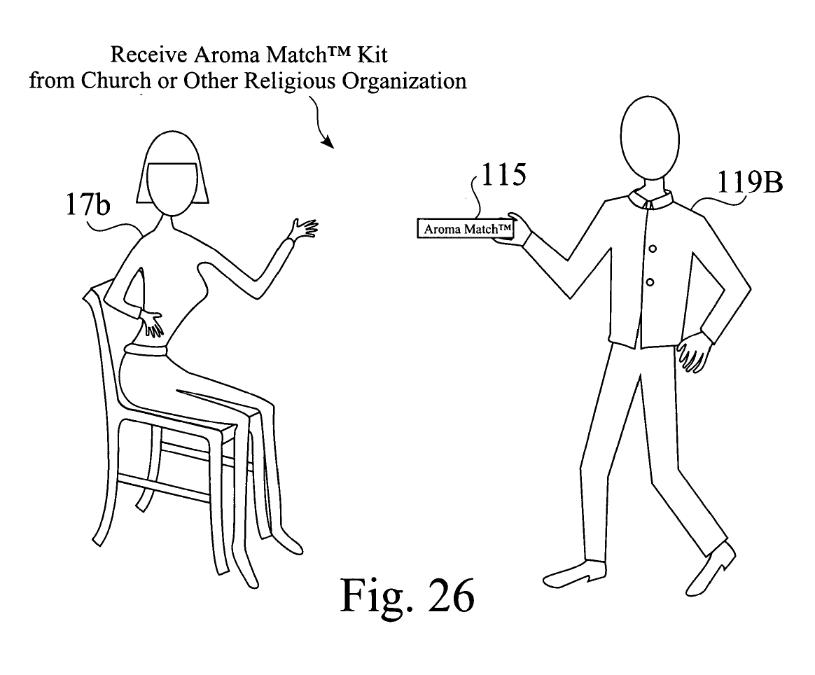

In yet another embodiment, FIG. 26 shows the woman 17b receiving a Test Kit 115 from a priest, minister, rabbi or some other religious leader or cleric 119B. In one embodiment, the invention is promoted by a religious or spiritual organization to promote good relationships and/or marriages.

In one embodiment of the invention, customers visit a website to supply information about themselves, and their ideal match. In this implementation of the invention, information is stored electronically in a computer database. In alternative embodiments, information about customers and their test results may be recorded in some other form of database, whether in electronic, paper or other means of media or storage.

In yet another embodiment of the invention, this database of information and/or records may be maintained by an introduction service, which may include a dating or matching service, or some other means for enabling, furnishing or assisting people find romantic or other matches. The introduction service may or may not utilize the Internet and/or electronic record keeping.

### II. The AromaMatch™ Test Kit

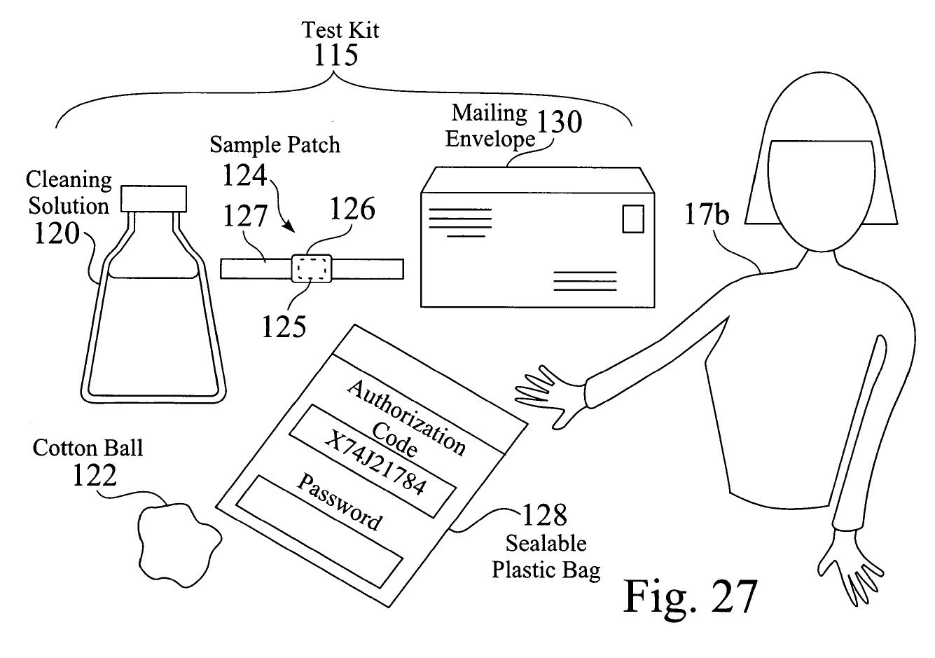

FIG. 27 portrays the woman 17b opening and removing the contents of the AromaMatch™ Test Kit 115. In one embodiment of the invention, the Test Kit 115 comprises:
cleaning solution or skin cleaner 120;
a cotton ball or other cleaning medium 122;
an odor-absorbing sample patch 124, which includes a portion of plaster 125 coated with an antibiotic 126 and portions coated with a skin adhesive 127;
a sealable enclosure 128, such as an envelope or bag; and
a mailing or shipping envelope or pouch 130.
   The skin cleaner 120 may comprise a liquid cleaning solution such as isopropyl alcohol, or any other, gel, solid, spray or substance that cleans and/or sterilizes a portion of the skin. The application of the skin cleaner 120 removes or neutralizes perfumes and other irrelevant smells.

The cleaning medium 122 is generally a small portion of material that is used to apply the skin cleaner 120 to the skin. In one embodiment, the cleaning medium 122 may be a cotton ball, wad, paper, piece of fabric or some other suitable application device.

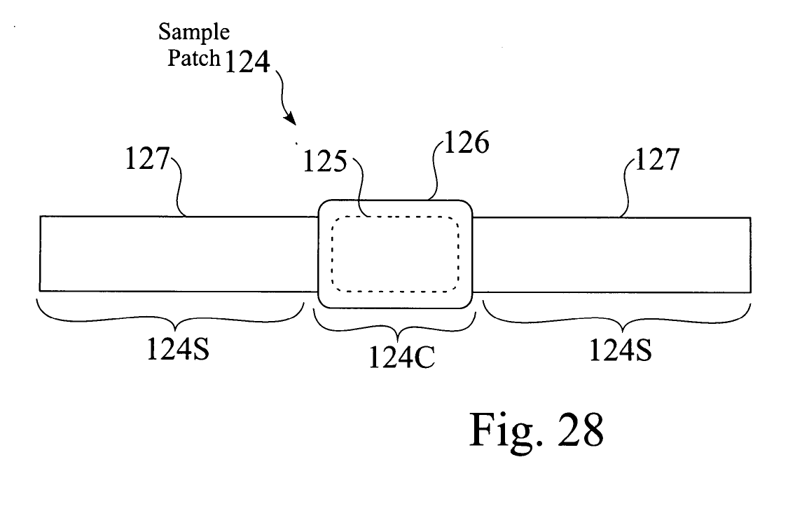

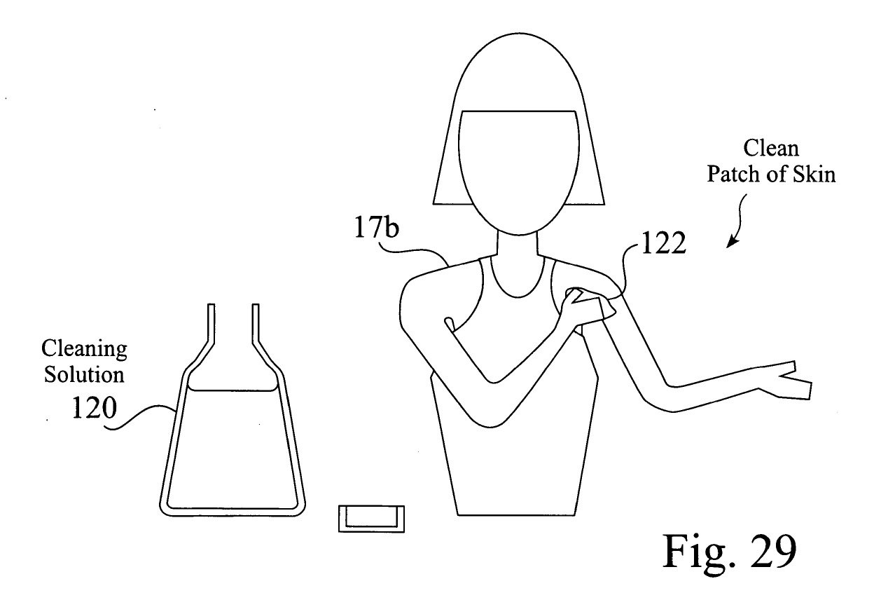

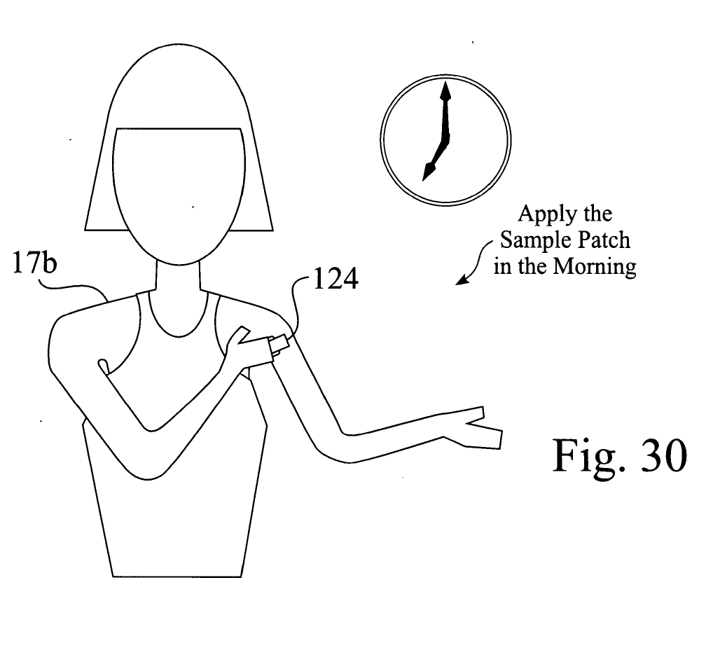

FIG. 28 furnishes an illustration of the sample patch 124, which comprises a small central area 124C with two outwardly extending strips 124S. The central area 124C is coated with a portion of plaster 125 which, in turn, has been coated with an antibiotic 126 or some other suitable agent that prevents bacterial growth which might modify the aroma. The strips 124S on either side of the plaster 125 are coated with an adhesive 127 that is suitable for adhering to the skin for a short period of time. Either side of the patch 124 may be coated with adhesive.

In one embodiment, the patch 124 resembles a conventional “Band-Aid® Brand” Adhesive Bandage, such as that manufactured and sold by Johnson & Johnson of New Brunswick, N.J. The patch 124 may be fabricated from plastic, cloth, paper or any other material that will maintain the plaster 125 in generally continuous contact with the skin. The plaster 125 is generally any material that will absorb and then hold an aroma which has been secreted by the skin. The plaster 125 may be composed of any substance that collects and stores an aroma. In this Specification and in the Claims that follow, the term “aroma” encompasses any scent, smell, odor or olfactory component that may or may not be actively or consciously detected, sensed or smelled by a person. In one embodiment of the invention, the plaster 125 is manufactured from any material that may be used as an odor-absorbing poultice.

The plaster 125 is designed so that it will collect enough aromas to provide a sample which may be reliably analyzed. The aromas captured by the plaster 125 must be able to survive for a period of time that is required for the patch 124 to be mailed to a laboratory.

After the Test Kit 115 is opened, the woman 17b cleans a patch of skin on her arm in preparation for applying the sample patch 124, as shown in FIG. 29. In a preferred embodiment, the patch is placed on the armpit. In FIG. 30, patch 124 has been attached to her skin. The patch 124 may be worn on any portion of the body which allows direct and intimate contact with the skin, and which enables a sufficient collection of body odor.

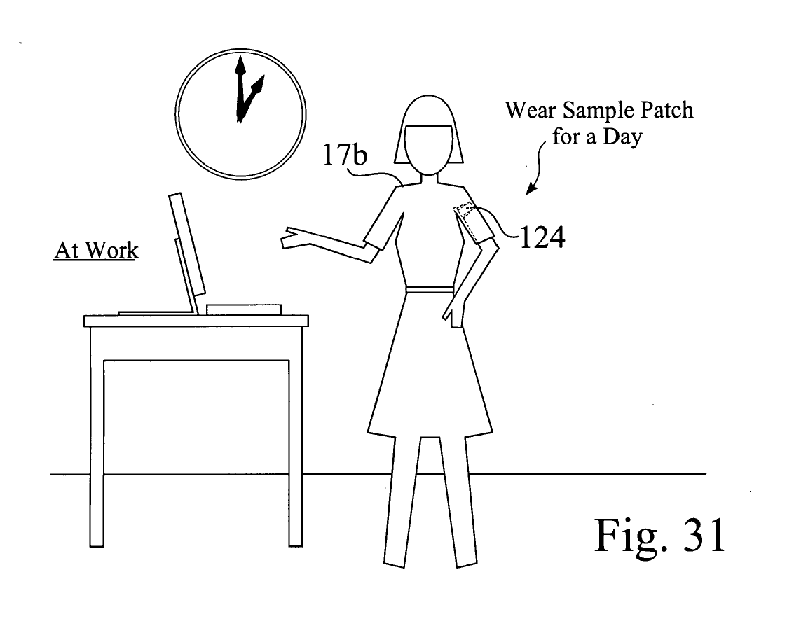

The woman wears the patch 124 all day, as shown in FIG. 31. The time that is required for the patch 124 to remain in place varies with the effectiveness of the plaster 125 and the sensitivity of the equipment used to analyze the patch 124. In one embodiment of the invention, the user is instructed to leave the patch 124 in place on the skin for at least eight hours. In some instances, the time that is required to wear to patch to obtain a good sample may take longer. One alternative method that may be used to collect a sample is using a simply wearing a shirt or some other article of clothing for an extended time, and then analyzing this worn article of clothing.

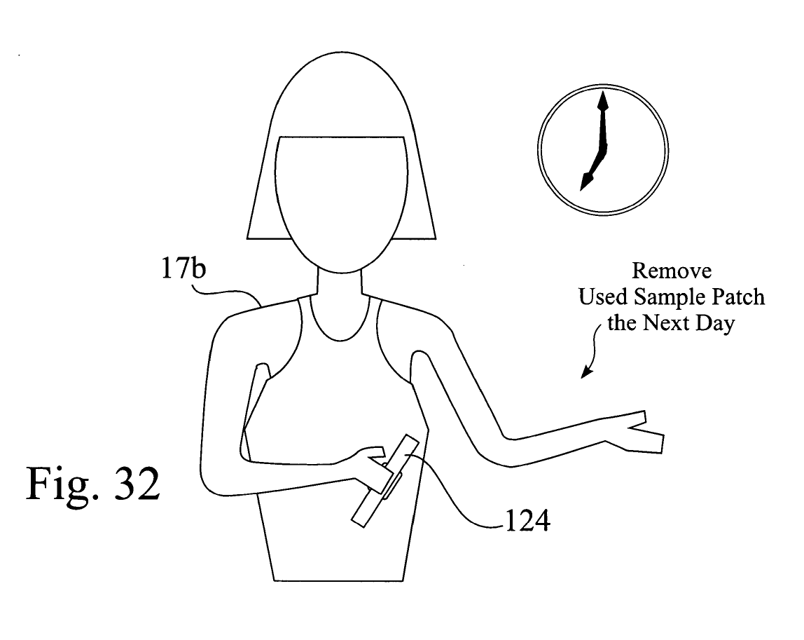

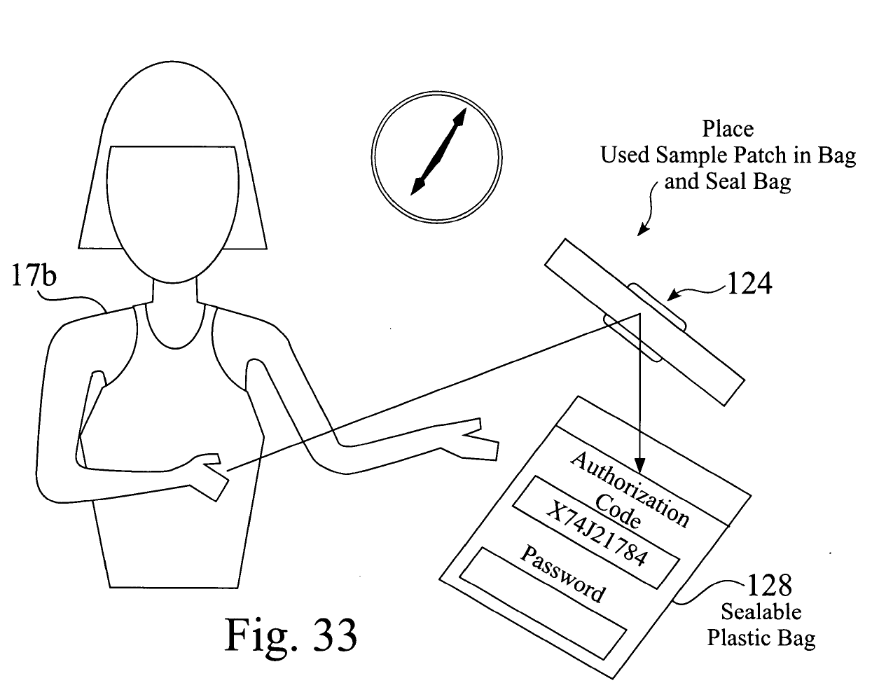

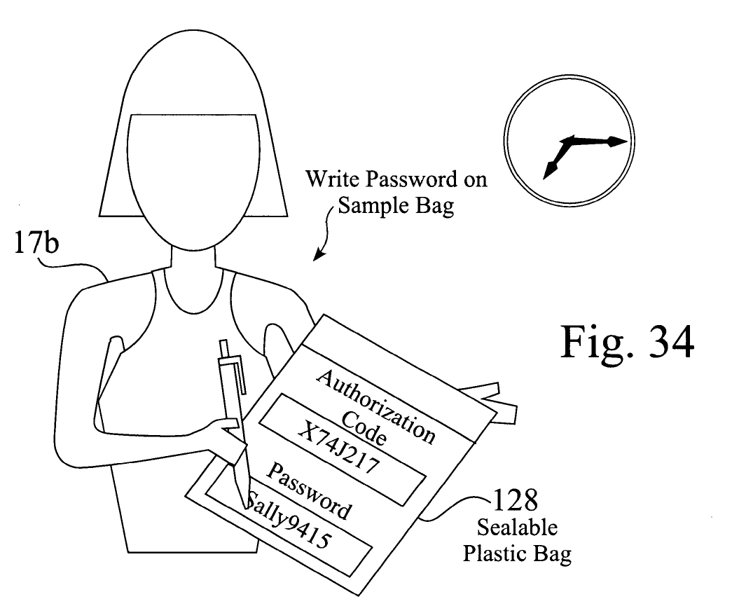

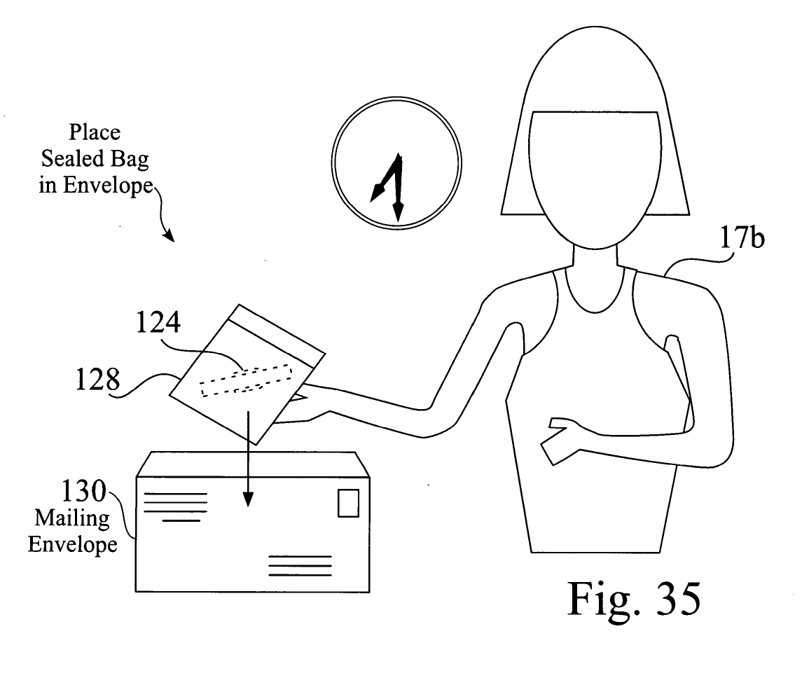

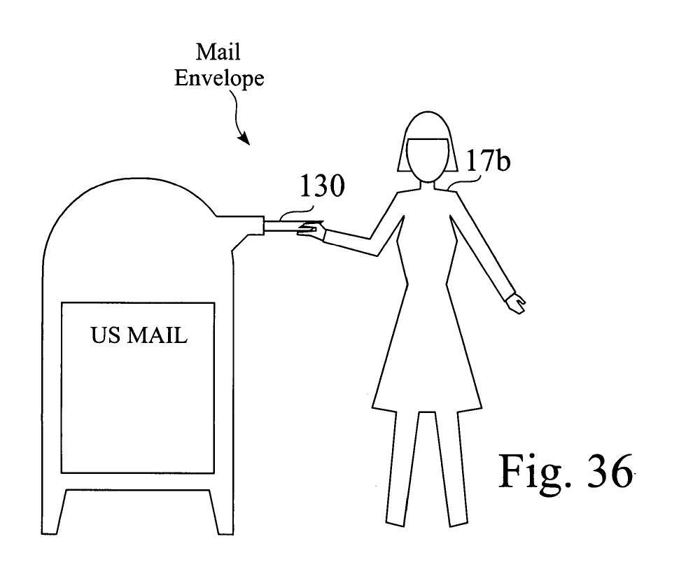

After wearing the patch 124 all day, the woman 17b removes the patch 124 later that evening, as shown in FIG. 32. After the patch 124 is removed, she then immediately places the patch 124 in the enclosure 128, as illustrated in FIG. 33. The enclosure is sealed 128 to prevent any degradation of the aromas stored in the plaster 125.

She then writes her username, password, code or some other identifying information on the bag 128, as shown in FIG. 34. This enclosure 128 is large enough to hold the sample patch 124, may be easily sealed against the intrusion of outside air by the user, and is generally an impermeable container or barrier that preserves the aromas imparted to the plaster 125 on the patch 124. In one embodiment of the invention, the enclosure 128 is a plastic bag with a compression seal, which is commonly known as a “zip-lock” or “slide-lock” closure. In one implementation, the bag 128 bears a pre-printed authorization code.

The patch 124 which stores the odor sample which has been sealed in the bag 128 is then placed in the mailing envelope 130, as shown in FIG. 35.

FIG. 36 portrays the customer posting the pre-addressed mailing envelope 130 which contains the worn patch 124 in the bag 128. This envelope 130 will convey the patch 124 to a laboratory where the plaster 125 will be analyzed. As an alternative, the patch 124 may be shipped to a laboratory using a courier. The patch 124 may also be delivered to a local laboratory, doctor's office or pharmacy for analysis. In a more advanced embodiment of the invention, the user may analyze the patch 124 using a home analysis kit.

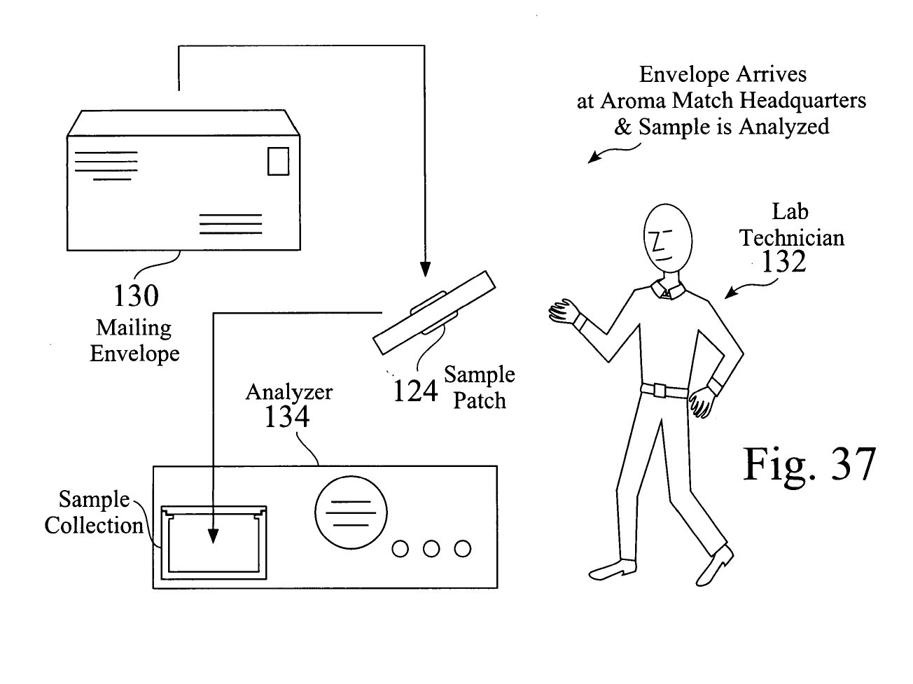

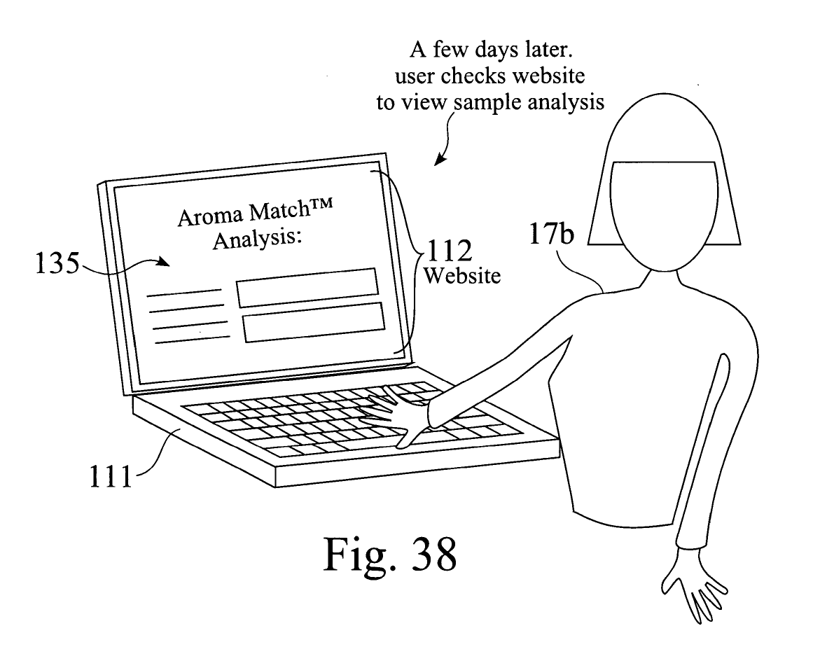

FIG. 37 shows a laboratory technician 132 using an analyzer 134 to determine the genetic attributes of the tissue sample that has been received from the customer 17b. In one embodiment, a probe from an analyzer 134 may be inserted into the bag 128, which will convey the aromas to a chamber where a chemical analysis is conducted.

Several devices and systems for analyzing a sample are currently available which may be used to implement the present invention. One device called an “Electronic Nose” has been described by The Lewis Group of The California Institute of Technology, and is based on readily fabricated, chemically sensitive conducting polymer films. According to information presented on their website:
“An array of sensors that individually respond to vapors can produce a distinguishable response pattern for each separate type of analyte or mixture. Pattern recognition algorithms and or neural network hardware are used on the output signals arising from the electronic nose to classify, identify, and where necessary quantify, the vapor or odors of concern. This response is much like the way the mammalian olfactory sense produces diagnostic patterns and then transmits them to the brain for processing and analysis.
“This approach does not require development of highly specific recognition chemistries, one for each of the many possible analytes of interest. Instead this approach requires a broadly responsive array of sensors that is trainable to the target signature of interest and then can recognize this signature and deliver it to the sensing electronics in a robust fashion for subsequent processing by pattern recognition algorithms. The Caltech electronic nose functions at atmospheric pressure, functions in a variety of ambients, exhibits near-real time detection, and has already been demonstrated to track vapors in air.
“The underlying principle of the Caltech electronic nose is extraordinarily simple. When a polymer film is exposed to a gaseous vapor, some of the vapor partitions into the film and causes the film to swell. In the electronic nose, this swelling is probed electrically because the sensor films each consist of a composite that contains regions of a conductor that have been dispersed into the swellable organic insulator. The vapor-induced film swelling produces an increase in the electrical resistance of the film because the swelling decreases the number of connected pathways of the conducting component of the composite material. The detector films can be formed from conducting polymer composites, in which the electronically conductive phase is a conducting organic polymer and the insulating phase is an organic polymer, or from polymer-conductor composites in which the conductive phase is an inorganic conductor such as carbon black, Au, Ag, etc and the insulating phase is a swellable organic material. The electrical resistance of the device is then read using simple, low power electronics.
“Any individual sensor film responds to a variety of vapors, because numerous chemicals will partition into the polymer and cause it to swell to varying degrees. However, an array of sensors, containing different polymers, yields a distinct fingerprint for each odor because the swelling properties over the entire array are different for different vapors. The pattern of resistance changes on the array is diagnostic of the vapor, while the amplitude of the patterns indicates the concentration of the vapor.”

### See: The Lewis Group, California Institute of Technology, Pasadena, Calif.

A second device that may be used to implement the present invention is called the “Cyranose,” and is described by Rodney M. Goodman, in his article entitled “The Electronic Nose.” According to Goodman:
“The technology uses sensors mixed with carbon black to make them conductive. The polymers swell with an odorant and their resistance changes. An array of different polymers swell to different degrees giving a signature of the odorant. This technology has been commercialized by Cyrano Sciences and a handheld electronic nose has been launched as a product.”

A third device that may be used to implement the present invention is described by Smiths Detection of Danbury, Conn., which produces and sells devices for identifying materials.

In FIG. 38, the customer 17b uses her computer 111 to visit the Internet Dating. Service website 112 to obtain the results of the laboratory analysis 135. In one embodiment, the analysis includes a listing of MHC alleles, MHC-determined peptides, MHC-odors or some other MHC-dependent profile. In an alternative embodiment, the results may be dispatched to the customer by regular mail or by e-mail.

In an alternative embodiment of the invention, the customer pays for the Test Kit 115 and the analysis when he or she obtains the results of the analysis.

In FIG. 39, the website reports the results 136 of a matching process that has been performed by comparing the customer's attributes to the attributes of a library of candidates. In one embodiment of the invention, the matching process correlates the set of self-describing attributes and the set of ideal-match attributes provided by the customer. Examples of attributes are supplied in Table Seven:

TABLE SEVEN

| Category | Examples of Attributes |
|---|---|
| Gender | Male, Female |
| Age | |
| Appearance | Handsome/Knockout, |
| | Attractive/Cute |
| Marital history | Single, Divorced |
| Residence location | |
| Height | Tall, Average, Short |
| Weight | |
| Hair Color | Blonde, Brunette, Redhead |
| Occupation & Income | |
| Religiosity | Yes/No; Denomination |
| Political preferences | Conservative, Liberal, None |
| Interests or hobbies | |
| Educational level | |
| Social Class Marker | |

The correlation process may involve comparing responses to individual preferences or predilections, or may involve more complex matching methods, such as those described in related U.S. patent application Ser. No. 11/881,153, entitled Searching Methods, which was filed on 24 Jul. 2007.

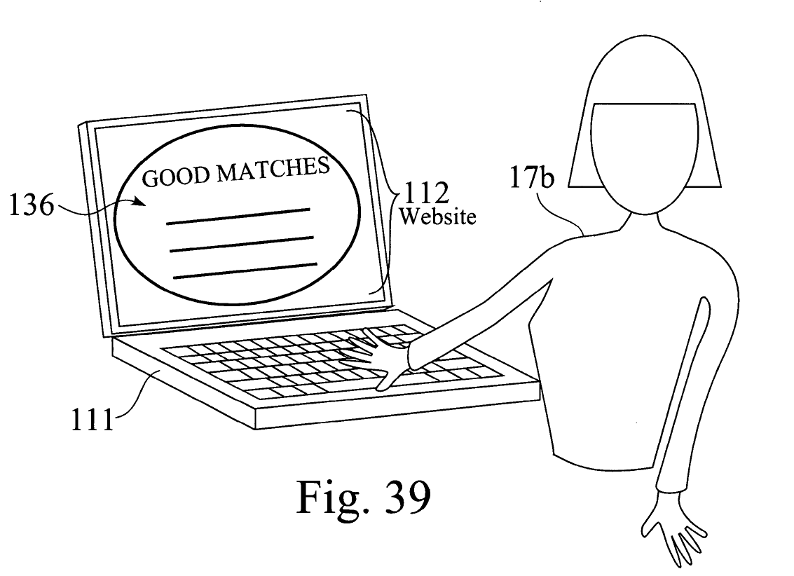

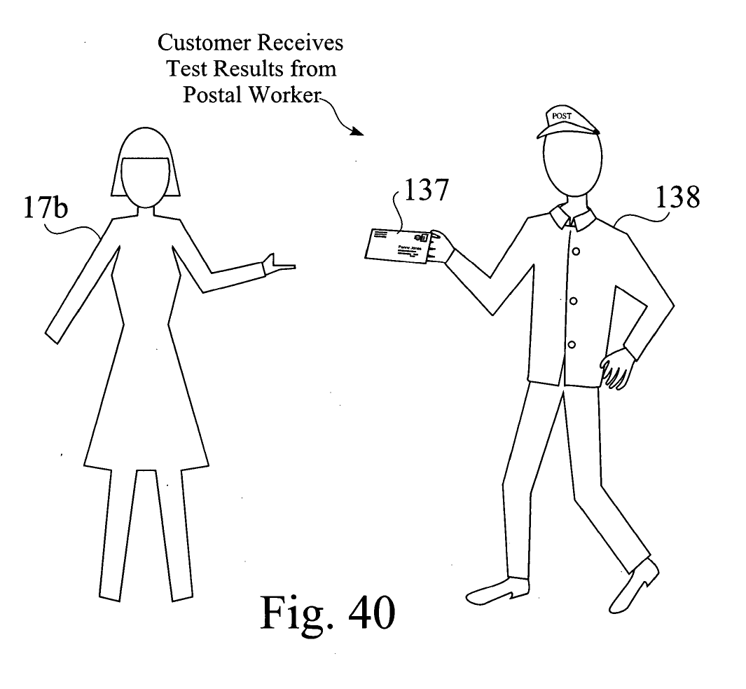

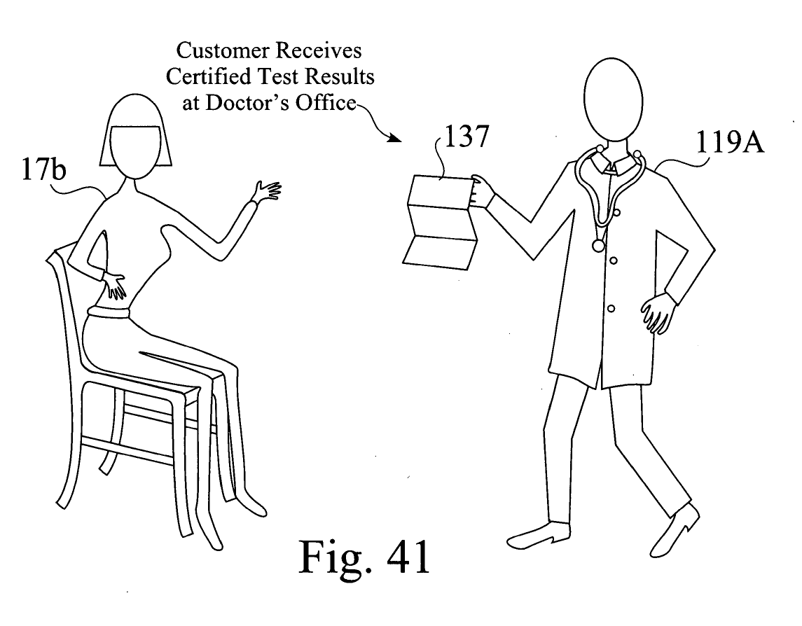

In FIG. 40, the customer 17b is shown receiving her test results 137 from a postal worker 138, while FIG. 41 shows the customer 17b receiving her test results 137 from a physician or other health care worker 119A in a doctor's office or clinic.

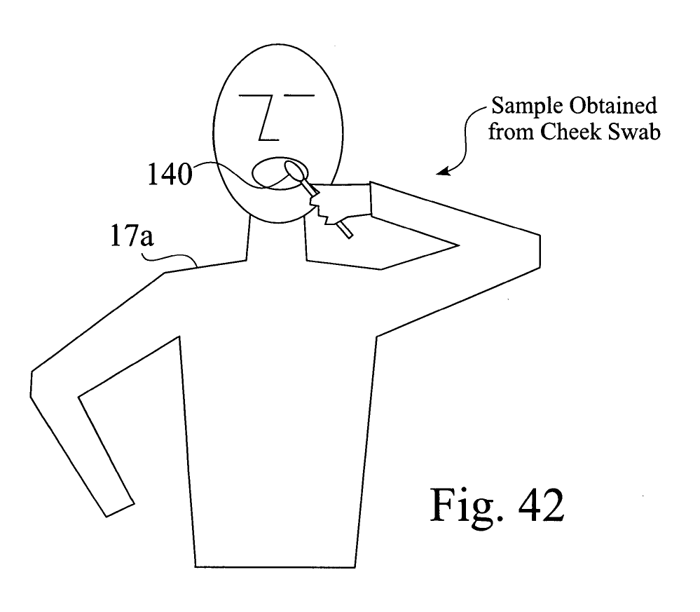

FIG. 42 reveals yet another alternative embodiment, in which a tissue sample 140 is obtained using a cheek swab. In other embodiments, a tissue sample may be obtained from any suitable bodily material or fluid, including, but not limited to, blood, saliva, exhaled breath, fingerprint, urine, hair, nail, or skin. One device that may be used to implement this portion of the present invention is produced and sold by DNA Genotek of Ottawa, Ontario, Canada, which produces and sells the Oragene™ DNA Self-Collection Kit, for collecting and preserving large amounts of DNA from saliva.

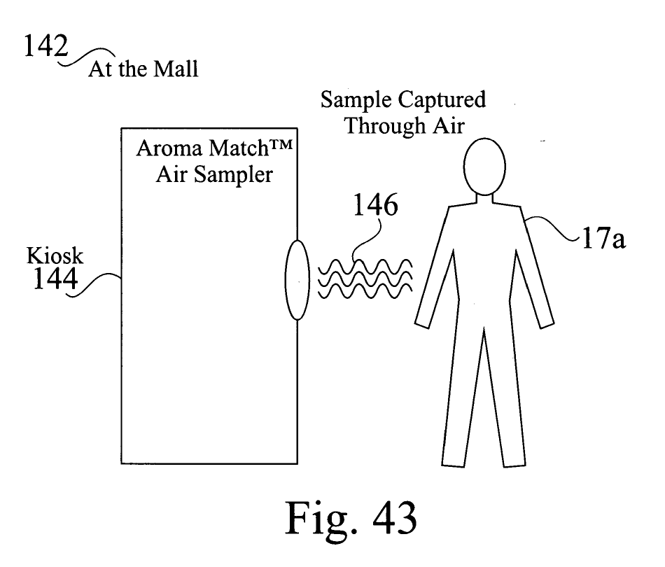

FIG. 43 exhibits an alternative embodiment, which collects a sample directly from the air 146 surrounding a customer 17a standing near a kiosk 144 that has been installed in a shopping mall 142. In yet another embodiment, a sample collecting tube may briefly be placed under a portion of a customer's clothing to obtain an air sample.

In an alternative embodiment of the invention, an automatic machine or device which accepts a DNA sample may be used to obtain an analysis without the intervention of a technician or clerk.

### III. One Specific Embodiment for Obtaining a Sample

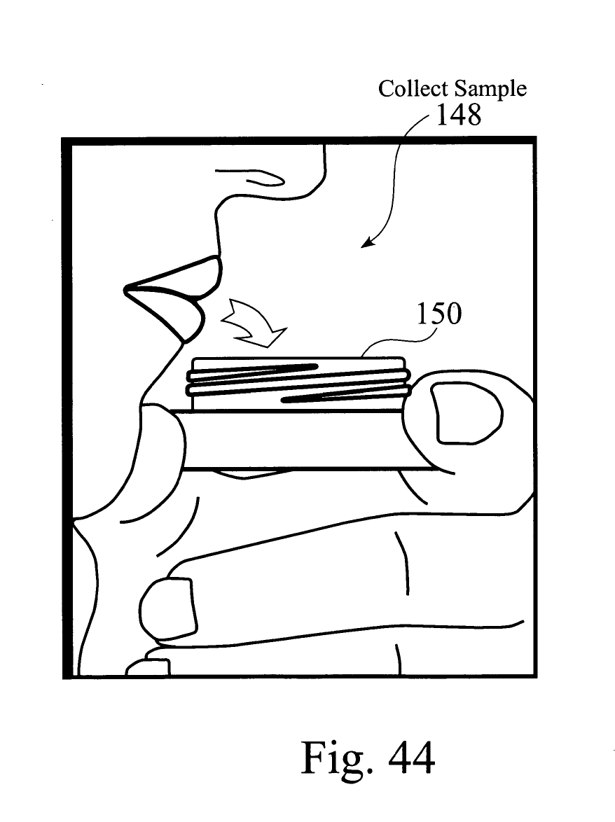

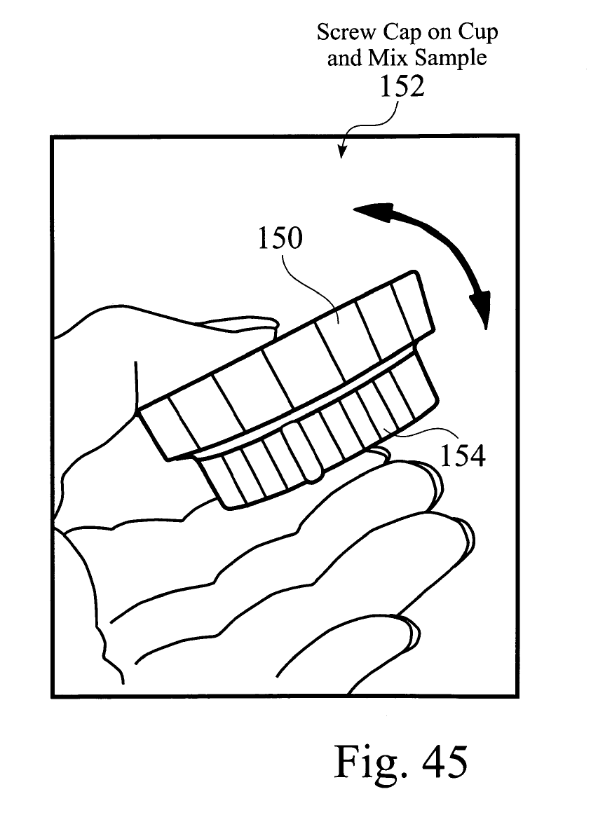

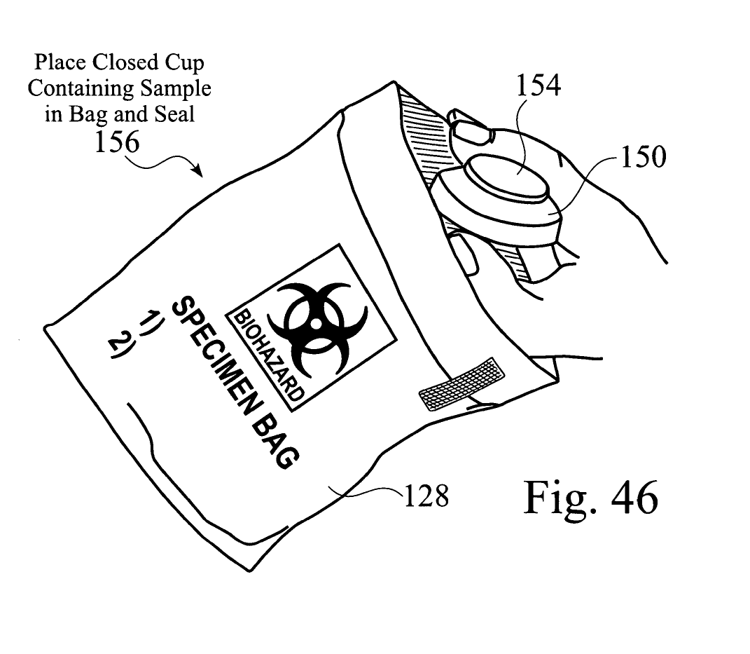

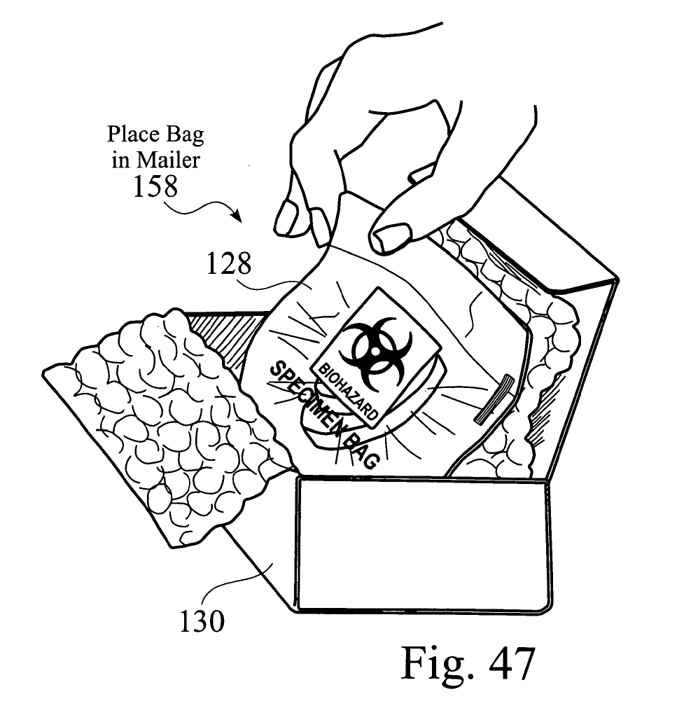

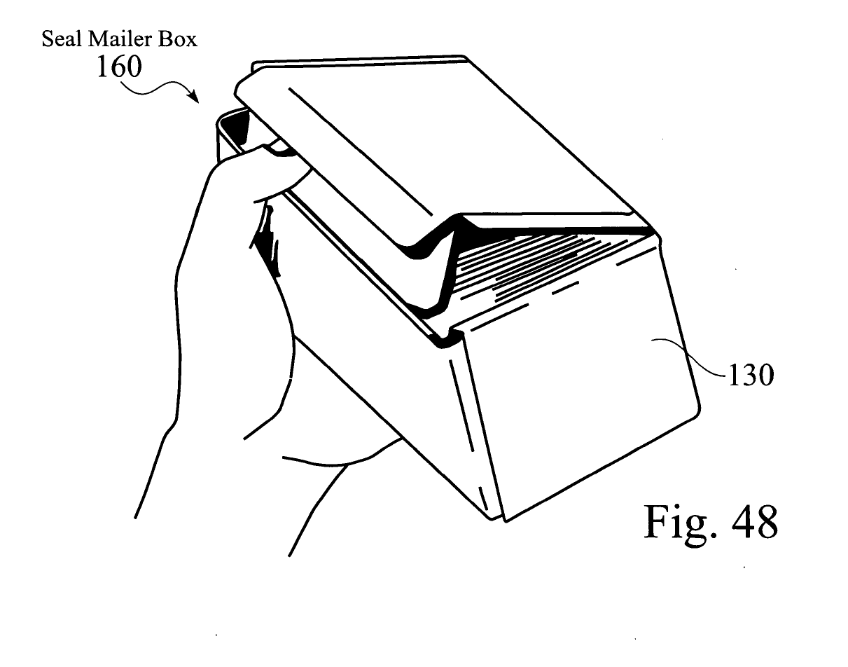

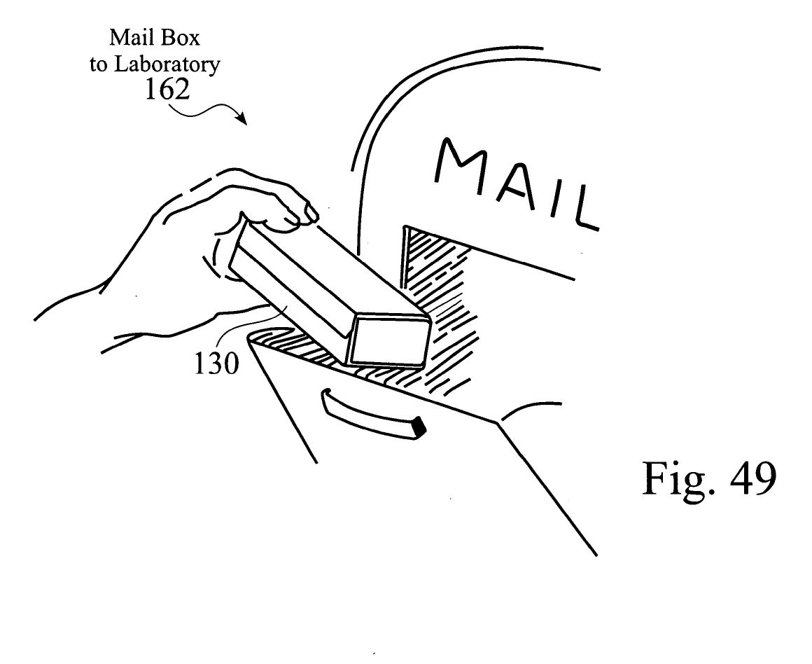

In one particular embodiment of the present invention, the customer 17 provides a saliva sample for analysis by a laboratory. FIG. 44 depicts the collection of a saliva sample 148 in a disc-shaped container or cup 150. In FIG. 45, a cap 154 is screwed on to the cup, and the sample is mixed 152. FIG. 46 illustrates the step 156 of placing the closed cup in a sample bag 128, and the bag 128 is sealed. FIG. 47 shows the step 158 of placing the sample bag 128 in a mailing box 130. FIG. 48 depicts the step 160 of sealing the mailing box, and FIG. 49 depicts the step 162 of mailing the box 130. More details concerning particular embodiments of sample collection and analysis that may be used to implement the present invention may be found by visiting the website for DNA Genotek, Inc. of Ottawa, Ontario, Canada.

The present invention may be implemented by obtaining any sample from a customer which may be analyzed to determine genetic characteristics.

### IV. Business Methods: Predicting a Good Match

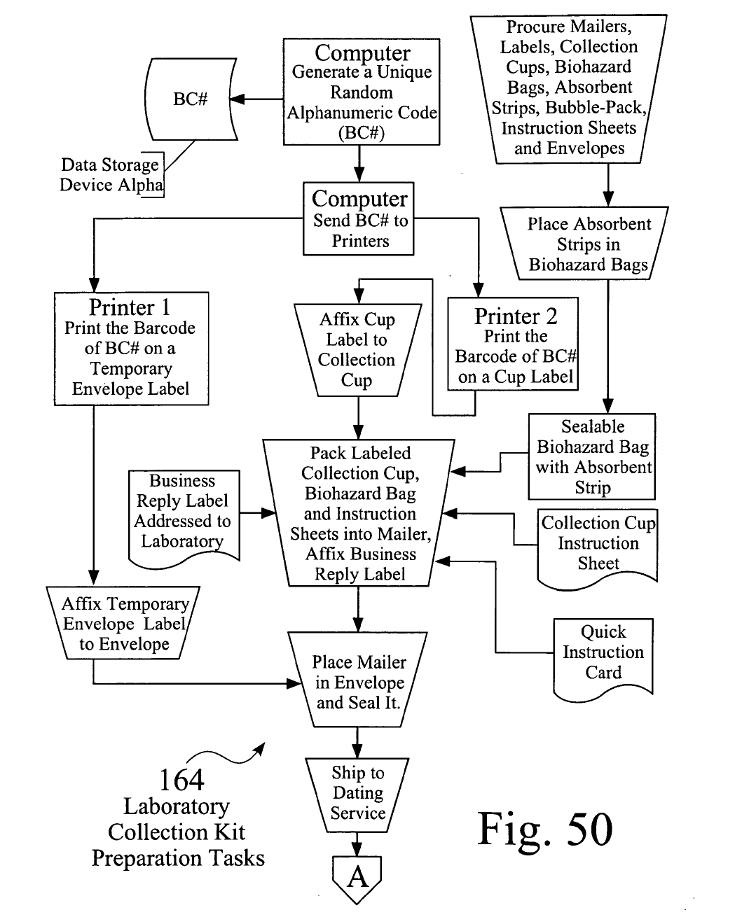

FIG. 50 is a flow chart 164 which illustrates laboratory collection kit preparation tasks.

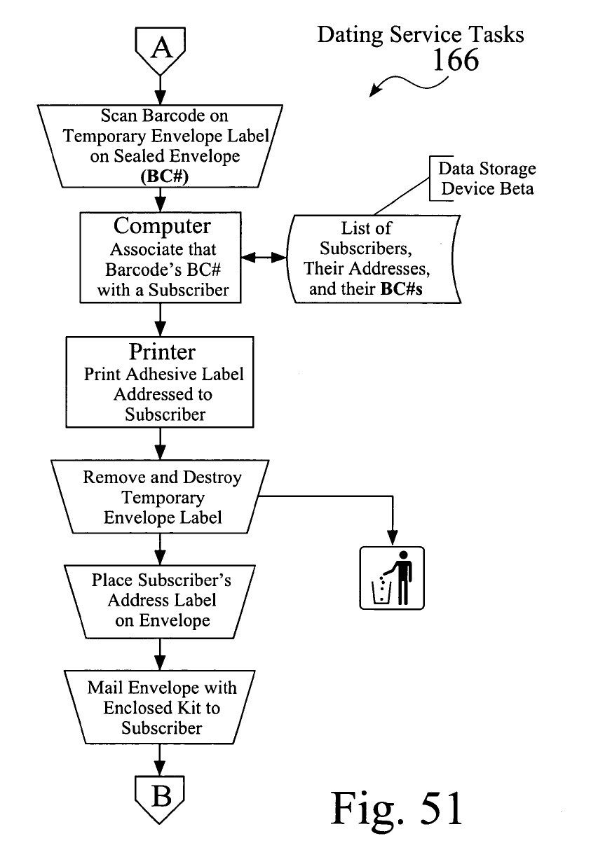

FIG. 51 is a flow chart 166 which illustrates dating service tasks.

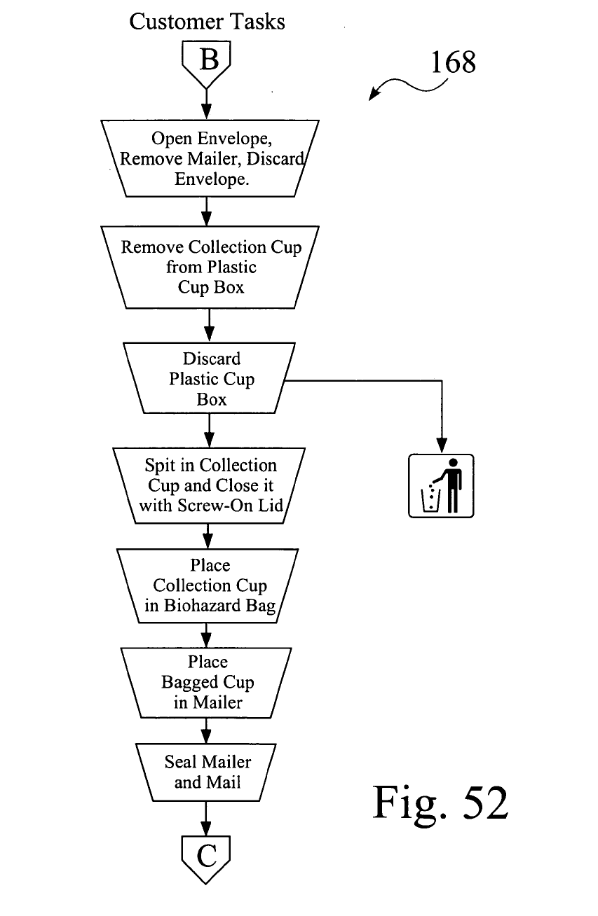

FIG. 52 is a flow chart 168 which illustrates customer tasks.

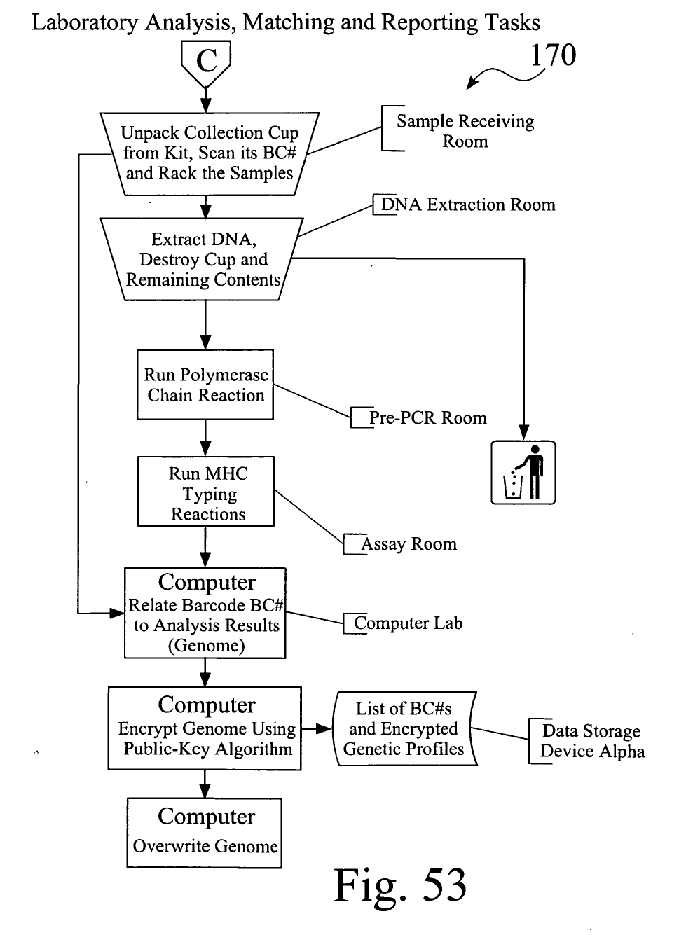

FIG. 53 is a flow chart 170 which illustrates laboratory analysis, matching and reporting tasks.

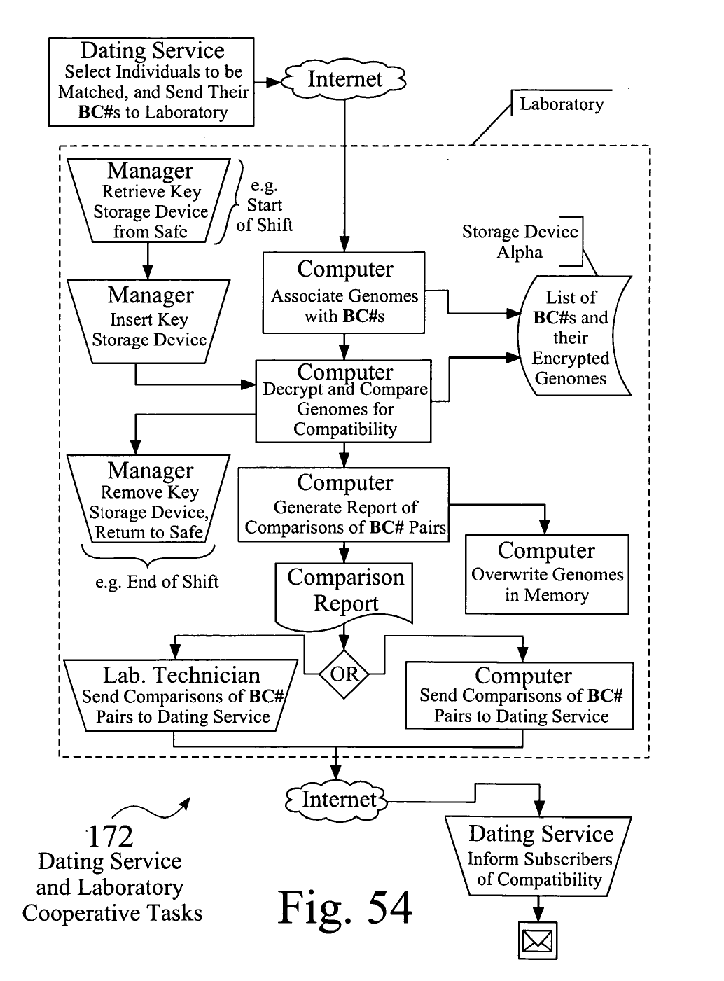

FIG. 54 is a flow chart 172 which illustrates dating service and laboratory cooperative tasks.

### V. MHC Biology

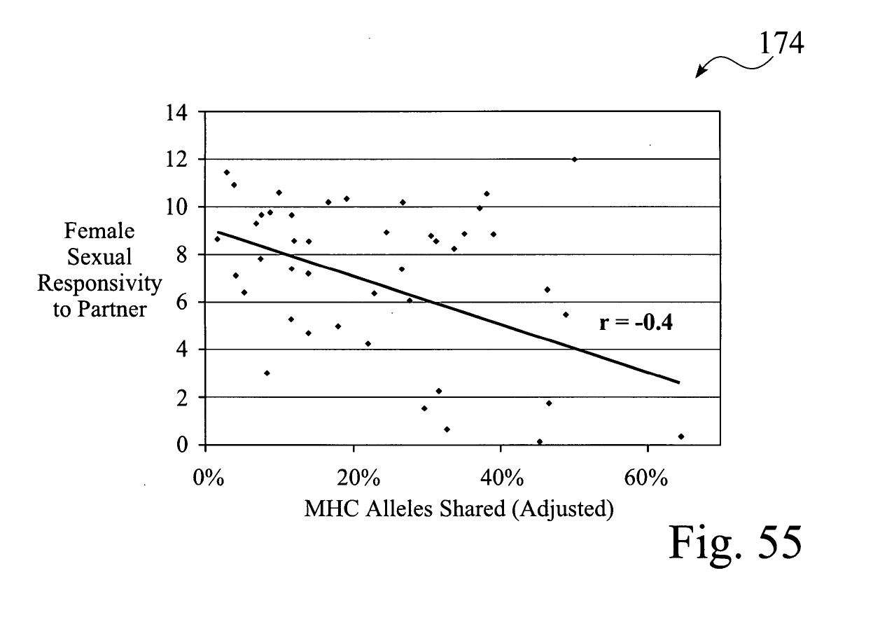

FIG. 55 is a graph 174 which plots experimentally measured human female sexual responsivity to another person on the y-axis, and the number of MHC alleles shared with that other person on the x-axis. The graph shows that a woman's sexual response, or responsivity, to a man is much higher if the man has MHC alleles which are different from her own. The greater the dissimilarity, the greater her response. The highest responsivity occurs when the proportion of shared MHC alleles is zero percent, while the lowest responsivity occurs when the proportion of shared MHC alleles approaches seventy percent.

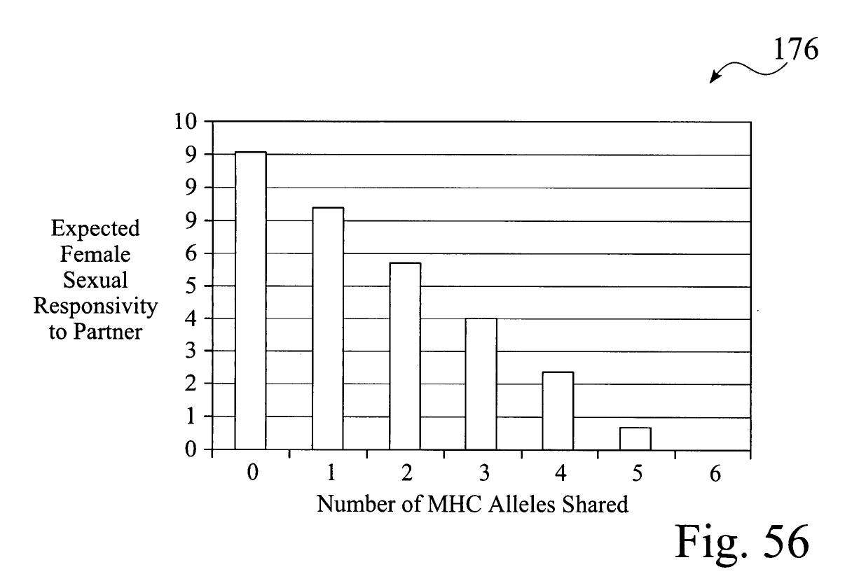

FIG. 56 depicts similar experimentally measured data in the form of a bar chart 176, and shows an expected female sexual responsivity to a partner along the y-axis, and the number of shared MHC alleles on the x-axis.

After a sample that has been obtained from a customer is received at a laboratory, the sample is processed to extract DNA. DNA is the chemical inside the nucleus of a cell that carries the genetic instructions for making living organisms. A cell is the basic unit of any living organism. It is a small, watery, compartment filled with chemicals and a complete copy of the organism's genome. Each cell contains a nucleus, which is the central cell structure that houses the chromosomes. Chromosomes are one of the threadlike “packages” of genes and other DNA in the nucleus of a cell. Chromosomes enclosed within the nucleus, which is, in turn, enclosed in the center of the cell.

Different species have different numbers of chromosomes. Humans have twenty-three pairs of chromosomes, forty-six in all: forty-four autosomes and two sex chromosomes. Each parent contributes one chromosome to each pair, so children get half of their chromosomes from their mothers and half from their fathers.

Part of the chromosome is called a gene. The gene is the functional and physical unit of heredity passed from parent to offspring. Genes are pieces of DNA, and most genes contain the information for making a specific protein.

A strand of DNA comprises a pair of helical ribbons attached by bases that resemble the rungs of a ladder. These bases are named adenine, thymine, guanine and cytosine. Sometime uracil is substituted for thymine. A section of one of the spiral sides of the DNA together with one of the bases comprises a nucleotide. Nucleotides are one of the structural components, or building blocks, of DNA and ribonucleic acid (RNA). A nucleotide consists of a base (one of four chemicals: adenine, thymine, guanine, and cytosine) plus a molecule of sugar and one of phosphoric acid.

Another set of chemicals that are important building blocks in humans are amino acids. Amino acids are the “building blocks” of proteins. There are twenty different kinds of amino acids in the human body. When two or more amino acids are bonded together, they form a peptide.

An allele is one of the forms of a gene at a particular location or “locus” on a chromosome. Alleles are specific sequences of base pairs that can be present at a given locus. For example, at the HLA-A locus in a particular individual, alleles in the A*01 and A*02 groups may be found. The “*” in the allele group name indicates that it was determined by DNA typing, as opposed to serological methods.

Different alleles produce variation in inherited characteristics such as hair color or blood type. In an individual, the dominant form of the allele is expressed, while the recessive form is not expressed. An exception to this rule is the case in which the genes at a particular locus are expressed codominantly, in which case they are both expressed.

In accordance with the present invention, small amounts of DNA are obtained from the sample submitted to a laboratory by a user who has submitted a saliva sample or skin scraping. In one embodiment of the invention, personnel at the laboratory cut the sample using a punch to make three separate disc-shaped pieces. These pieces are each placed in a different test tube. All the pieces are washed several times with chemicals that purify the sample on each piece. After washing, each piece is dried in its tube.

In an alternative embodiment, if a saliva sample is obtained from the customer, the saliva is poured directly into three separate test tubes, washed and then the DNA analysis is performed.

When DNA is analyzed, a laboratory technician looks at particular places or “loci,” (which are the positions in a chromosome in which specific genes are known to occur) to determine the particular allele (variation of the gene). Previous research has determined that every person has a characteristic sequence of genetic material (allele) that resides at each of his or her genetic loci.

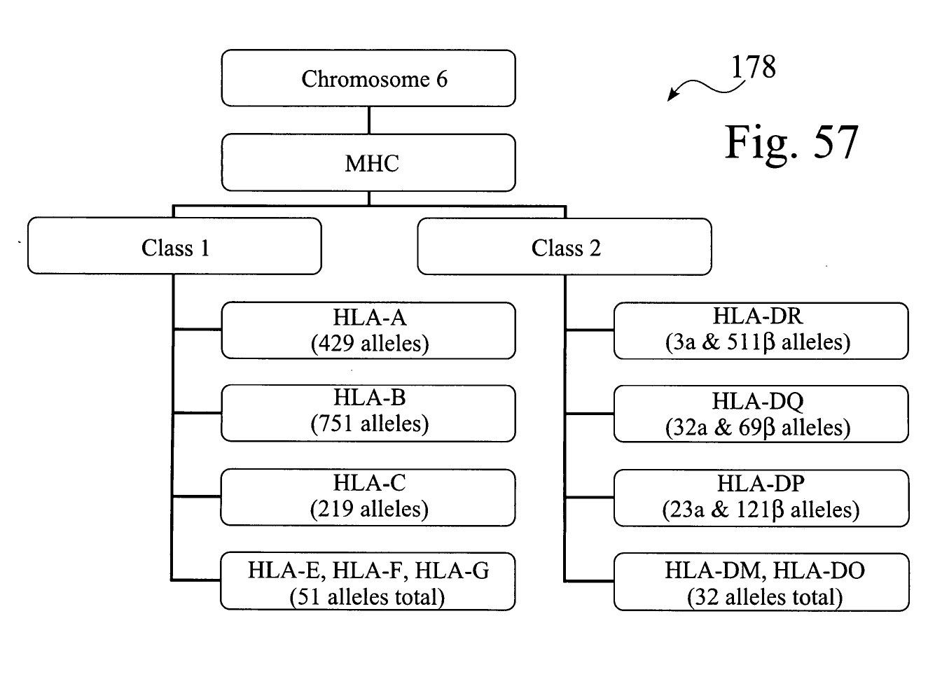

The laboratory technician basically examines particular sets of alleles that are found at a particular group of loci on a particular chromosome. To match alleles in the MHC region of the genome, the technician “takes an inventory” of the genetic material in the MHC region on Chromosome 6. Parts of the MHC are broken down into smaller groups of genetic material, and are given names. In one embodiment, the parts of the MHC that need to be inventoried are named “HLA-A,” “HLA-B” and “HLA-DR [symbol] 1.” These parts of the MHC are on a particular region of a particular chromosome. All these relationships 178 are illustrated in FIG. 57.

The term “allele groups” are also known as “2-digit alleles” and “2 alleles.” “High resolution alleles” are also known as “4-digit alleles” and “4 alleles.”

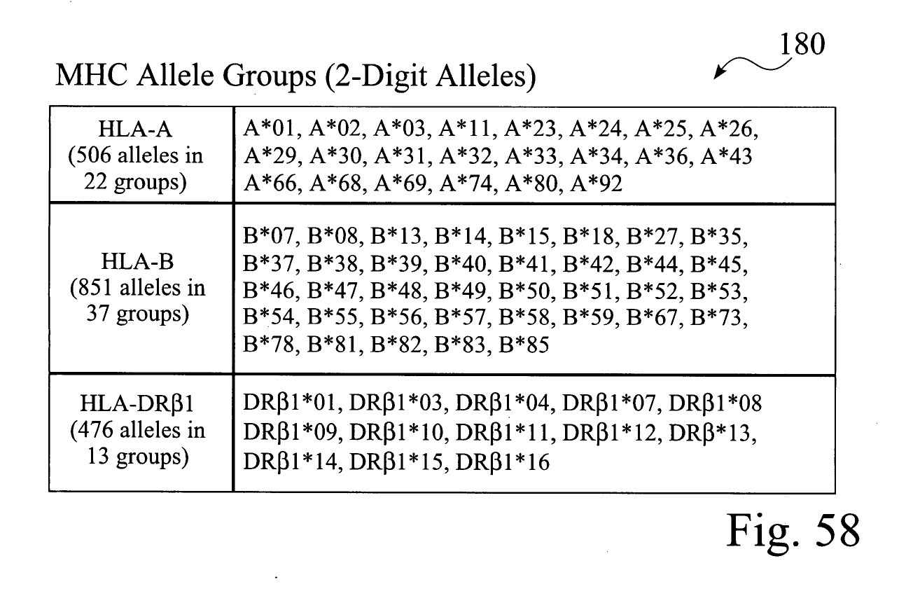

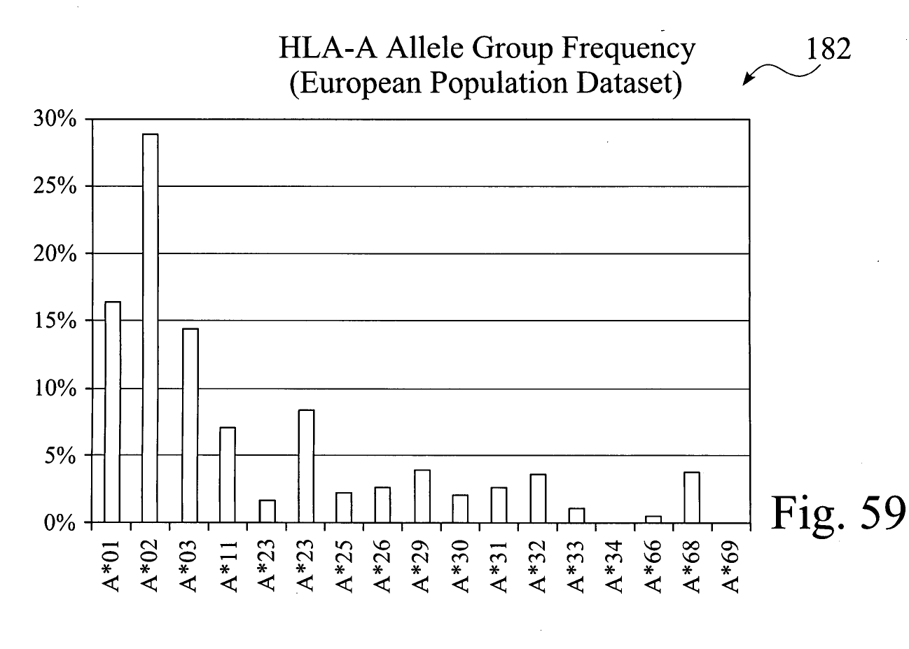

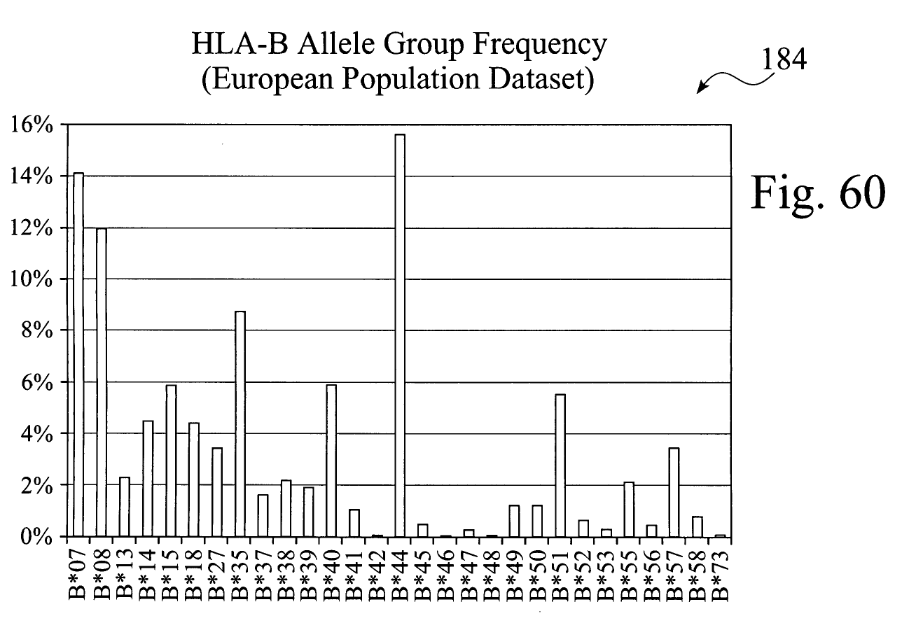

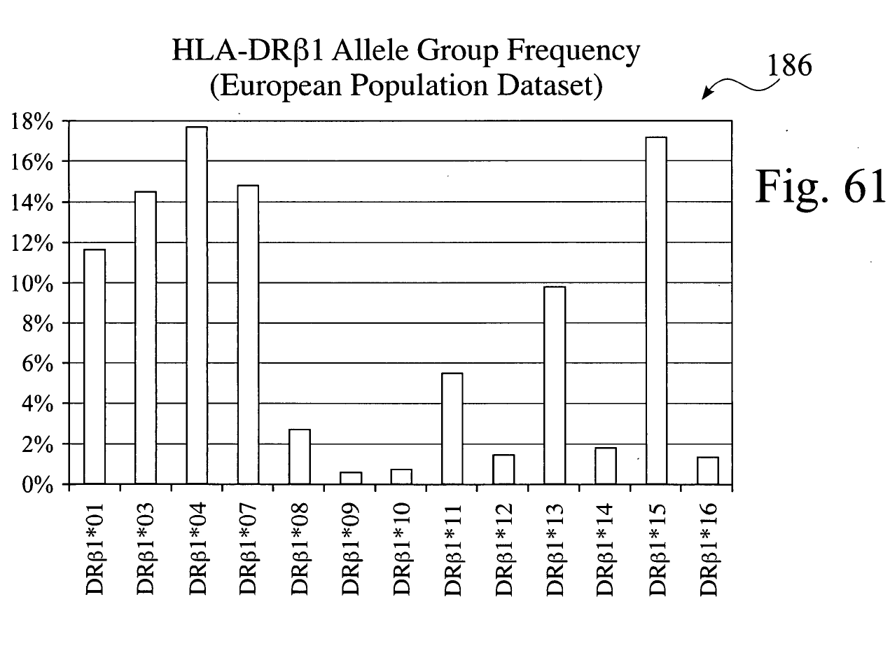

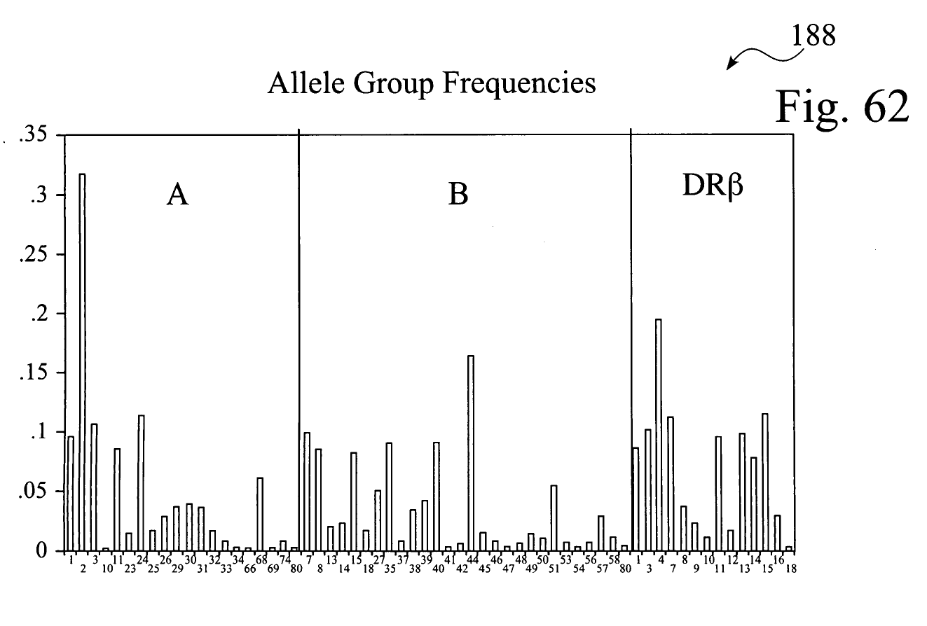

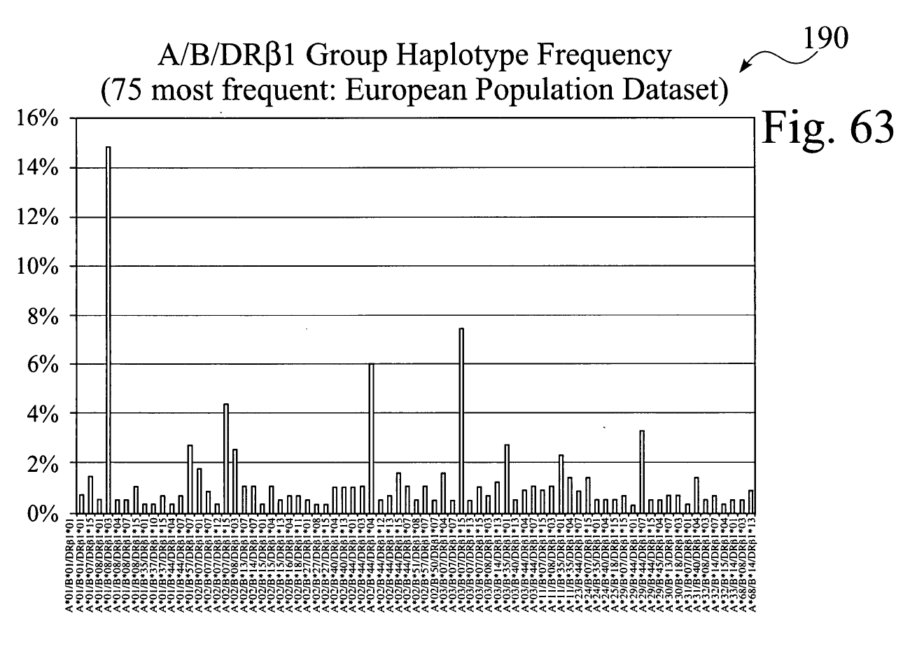

There are 21 HLA-A allele groups, 37 HLA-B allele groups, and 13 HLA-D [symbol] 1 allele groups. The various MHC Allele Groups 180, such as “A*01,” “A*02” and “A*03” are presented in FIG. 58. FIG. 59 depicts HLA-A Allele Group Frequency 182. FIG. 60 depicts HLA-B Group Frequency 184. FIG. 61 depicts HLA-DR [symbol] 1 Group Frequency 186. FIG. 62 depicts Allele Group Frequencies 188. FIG. 63 depicts A/B/DR [symbol] 1 Group Haplotype Frequency 190.

The sequence-specific oligonucleotide probe (SSOP) method is used. The basis of this method is HLA locus-specific amplification by polymerase chain reaction (PCR), and the subsequent probing of the resulting product by SSOP. A battery of probes is required. The pattern of reaction to these probes distinguishes the HLA alleles.

For each sample, the laboratory uses PCR for HLA locus-specific amplification at HLA-A, HLA-B, and HLA-DR [symbol] 1. Each of the three PCR amplifications results in a product. Each of the three products is then tested with a battery of probes. The HLA-A amplified product is tested with 12 probes at exon 2 and 16 probes at exon 3. The HLA-B amplified product is tested with 18 probes at exon 2 and 18 probes at exon 3. The HLA-DR [symbol] 1 amplified product is tested with 25 probes at exon 2. These are sufficient numbers of probes so that the reaction patterns will distinguish the HLA allele groups (2-digit alleles), for example, A*02.

After all the genetic codes that are contained on a sample piece is identified, this information is entered into a database along with the personal information and match preferences of the customer who submitted the sample.

Previous scientific research has determined that a woman's attraction to a particular man and her sexual response to him is based on the correlation between the alleles in the woman's MHC, and in the man's MHC. Specifically, a woman and a man who have different MHC genetic codes are more sexually compatible than a man and a woman who have similar MHC genetic codes.

So, when the lab technician takes an inventory of all the different allele groups (2-digit alleles) in a user's DNA sample, the technician is creating an identification or map of the person who submitted the sample. By comparing this identification or map with that of a different person, a technician can predict which other people will be attractive and sexually responsive to the customer, all based on the genetic code of each individual. In addition to the HLA-A, HLA-B and HLA-DR [symbol] 1 loci specified above, genetic information from other loci on Chromosome 6 or any other chromosome may be used to enhance a match.

In alternative embodiment of the invention, genetic attributes are determined by analyzing serologically typed HLA antigens. While “allele groups” are determined by genetic testing, such as PCR-SSOP, HLA antigens are determined by serological, or blood reaction, testing. Serological typing provides approximately the same resolution as “2-digit alleles.” It cannot provide the higher resolution comparable to “4-digit alleles.”

More detailed information concerning this analysis may be found in Methods in Molecular Biology, Vol. 210: MHC Protocols, edited by S. H Powis and Robert W. Vaughan, Humana Press Inc., Totowa, N.J., 2003. (See Chapter 5, “PCR-Sequence-Specific Oligonucleotide Probe Typing for HLA-A, -B, and -DR, by Derik Middleton and F. Williams). Another useful publication is Histocompatibility Testing, edited by Jeffrey L. Bidwell and Cristina Navarrete, Imperial College Press, 2000. (See Chapter 6, “PCR-SSOP Typing” by D. Middleton.) These publications explain how to type the MHC loci of interest using a two-tier system. The first level of resolution determines the allele group (2-digit alleles), and the second level uses this knowledge to determine the allele subgroup (4-digit alleles). Alleles in the MHC region may also be identified by the antigens produced by the proteins manufactured in the cells, using the “blueprint” provided by the allele. These Human Leucocyte Antigens (HLAs) may be typed by the complement-dependent lymphocytotoxicity.

HLA typing can be performed by the complement dependent lymphocytotoxicity reaction (serology). Live peripheral blood mononuclear cells are required for this assay (CD8+ T-cells and/or CD19+). B-cells are purified from whole blood, and incubated against a panel of antibodies with specificity against polymorphic epitopes expressed on HLA-A and -B proteins. In the presence of complement cells expressing HLA proteins which react with a particular antibody are lysed, allowing these damaged cells to uptake a stain which is detected by fluorescent microscopy. The pattern of negative and positive reactions is scored and interpreted to give an HLA serological type. HLAs may also be identified by their odor, and it is this method that humans and other mammals use for mate selection and personal identification, and by the electronic odor sensing process described above.

Additional information concerning HLA analysis may be found in Histocompatibility Testing, edited by Jeffrey L. Bidwell and Cristina Navarrete, Imperial College Press, 2000. (See Chapter 1, “HLA Typing by Alloantibodies and Monoclonal Antibodies” by G. M. Th. Schreuder; and Chapter 2, “Screening for HLA-Specific Antibodies” by C. Brown and C. Navarrete.) These publications explain how to type the MHC loci of interest using antibody reactions.

As genome sequencing has become less expensive, there has been a great deal of interest in pairing variations in certain genes with variations in behavior (“Molecular Psychiatry”). This science is still young: the following are recent discoveries. This invention can be used to refine its relationship-predicting service by including some or all of these genetic loci:

The brain neuropeptide arginine vasopressin (AVP) is a pituitary hormone which regulates blood pressure and kidney function in mammals. Studies of voles (one of the few non-human mammals that exhibit pair-bonding) found that AVP exerts a strong influence on their pair-bonding. This work has recently been extended to humans, and has revealed an association between one of the alleles of the AVPR1a gene (this gene codes for cellular receptors for AVP) and traits reflecting pair-bonding behavior in men, including partner bonding, perceived marital problems and marital status. The study also shows that it affects marital quality as perceived by their spouses. See Walum, Hasse et al., “Genetic variation in the vasopressin receptor 1a gene (AVPR1a) associates with pair-bonding behavior in humans,” Proceedings of the National Academy of Sciences, Vol. 105, No. 37, Sep. 16, 2008).

Oxytocin is a hormone “ . . . which seems to modulate a wide range of sexual and social behaviors from social recognition, pair bonding, mate guarding and parental care in rodents, to love, trust or fear in humans.” Certain alleles of the CD38 gene lead to impaired nurturing behaviors, social amnesia (failure to recognize others) and is suspected of causing “ . . . some forms of impaired human behavior in the spectrum of autism disorders.”See Jin, Duo et al., “CD38 is critical for social behavior by regulating oxytocin secretion,” Nature, Vol. 446, pp. 41-45 (2007).

Variations in a dopamine receptor gene (DRD4, on Chromosome 11 in humans) contribute to individual differences in human sexual behavior: desire, arousal and sexual function, and in particular predicts overall sexual interest. Studies that have shown this effect in animals have now been extended to humans and show similar results. See Ben Zion, I Z, et al., “Polymorphisms in the dopamine D4 receptor gene (DRD4) contribute to individual differences in human sexual behavior: desire, arousal and sexual function,” Molecular Psychiatry Vol. 11, pp. 782-786 (2006), and Pearson, Helen, “Sexual desire traced to genetics,” Nature Online, doi:10.1038/news060529-6 (Published online 31 May 2006).

Recently-published work finds that variations in the ER (Chromosome 6) locus predict psychoticism, neuroticism, non-conformity and extraversion in women, including sexual behavior. See Westberg et al., Association between a dinucleotide repeat polymorphism of the estrogen receptor alpha gene (ERα) and personality traits in women, Molecular Psychiatry 8, Pages 118-122 (2003).

A more detailed description of matches using the information obtained by analyses of MHC, HLA and other genome loci such as those described above, as well as the Attributes listed in Table Seven, may be found in a related Pending U.S. patent application, U.S. Ser. No. 11/514,285, entitled Matching System, which was filed on 30 Aug. 2006.

VI. Finding Good Matches with a MateFinder™

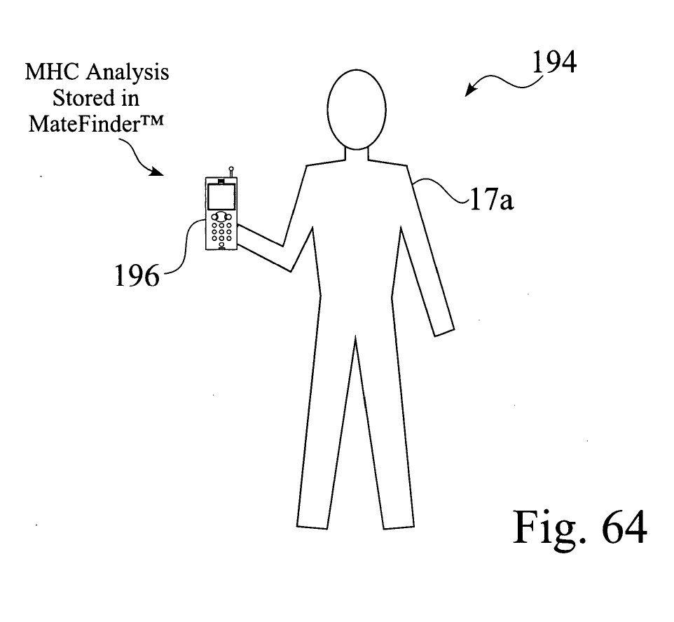

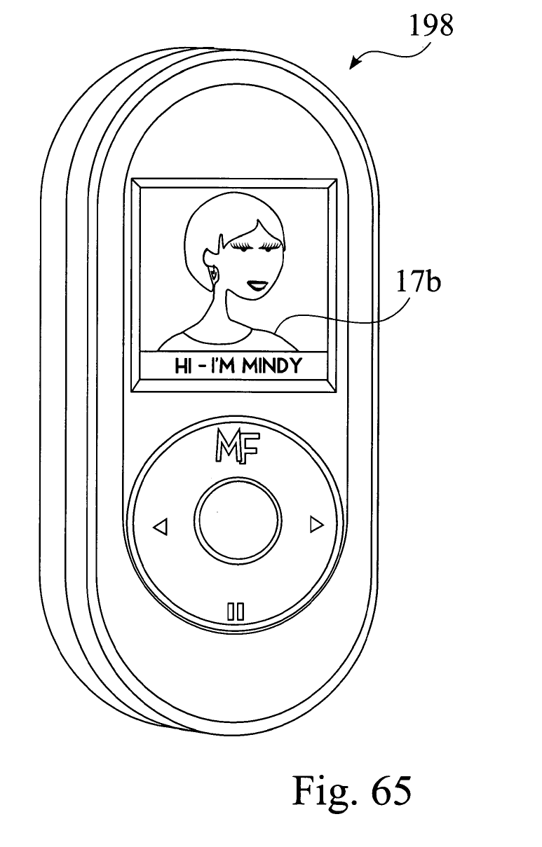

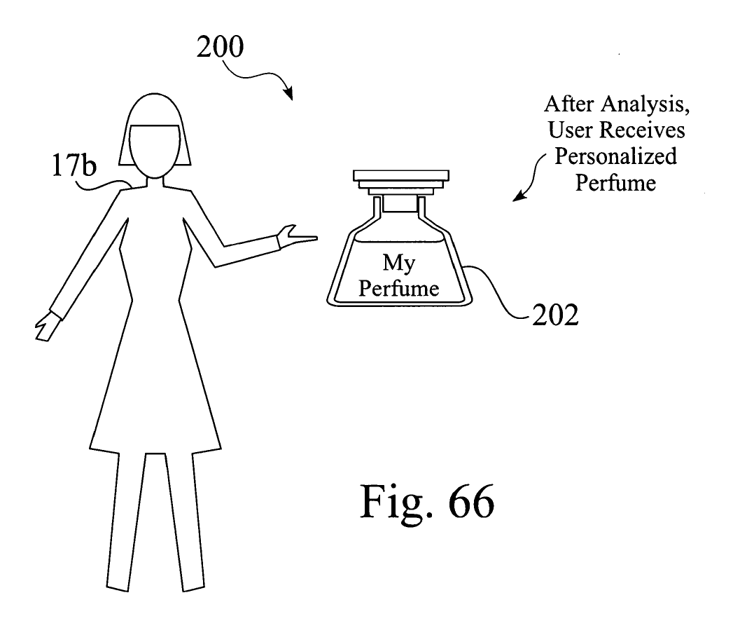

FIG. 64 shows the step 194 of a customer using a MateFinder™ device 196 which has been programmed with his genetic attributes, as determined in accordance with the present invention. FIG. 65 offers a detailed view of one embodiment of a MateFinder™ device 198.

In one particular embodiment, the MateFinder™ comprises a radio and a microprocessor with a non-volatile memory, such as a static random-access memory (RAM). Information that describes both the user and the ideal match can be written to the non-volatile memory. The radio automatically and periodically broadcasts a “seeking signal” over a short range. When the seeking signal is received by another MateFinder™, it is analyzed to determine the degree of correlation with the receiver's preferences. If the degree of correlation exceeds a preset minimum, the sender, the receiver, or both are alerted.

Another embodiment combines the MateFinder with a network radio or device, such as a cellular or Voice over Internet Protocol (VoIP) telephone or some other suitable device to provide communications over a wireless network. This combination enables voice calls, text-messaging, instant messaging, e-mails and Internet browsing. The user may also arrange to transfer gifts of music, photographs, video clips and other matter purchased from a third party. The MateFinder may be connected to a network using Wi-Fi, Wi-MAX, UltraWide Band (UWB) radio or any other suitable wireless system. The MateFinder may also communicate over a wired network such as the conventional telephone network, the Internet or may use VoIP.

In another embodiment of the invention, the MateFinder is programmed with information concerning the genetic attributes of a number of individuals. Romantic matches are suggested by correlating the genetic attributes of different individuals. These genetic attributes are first determined by testing tissue or fluid samples.

A more detailed description of this aspect of the present invention may be found in a related Pending U.S. patent application, U.S. Ser. No. 11/514,285, entitled Matching System, which was filed on 30 Aug. 2006.

### VII. Benefits of the Invention

### Reducing Consanguinity

The present invention includes a method for selecting candidates for a relationship based on diversity in the Major Histocompatibility Complex (MHC) region of their genomes. This method of the invention reduces the risk of couples' producing children with birth defects that may arise from parents who are too closely related, and who may carry the same deleterious recessive gene. When two individuals share similar genetic characteristics, their relationship may be described as “consanguineous.” According to Wikipedia, the terms consanguineous and consanguinity indicate a relationship in which two persons are “of the same blood or origin; specifically: descended from the same ancestor.”

It has been known since prehistoric times that closely-related members of a mated pair, be they plants, domesticated animals or humans, are at a much higher risk of having offspring with birth defects and other weaknesses, or to lose their progeny as embryos or fetuses. It has also been recognized since antiquity that outbred offspring tend to have better health and general fitness. This is the origin of the term, “hybrid vigor,” or heterosis.

This effect also drives a major facet of human ethical behavior. Few human transgressions are viewed with as much odium is incest; all known cultures have strong taboos prohibiting this activity. Inbreeding avoidance is also seen in many non-human species, including invertebrates.

The deleterious effects of inbreeding are well-explained in Wikipedia:
“Two leading hypotheses explain the genetic basis for fitness advantage in heterosis.
“The overdominance hypothesis implies that the combination of divergent alleles at a particular locus will result in a higher fitness in the heterozygote than in the homozygote. Take the example of parasite resistance controlled by gene A, with two alleles A and a. The heterozygous individual will then be able to express a broader array of parasite resistance alleles and thus resist a broader array of parasites. The homozygous individual, on the other hand, will only express one allele of gene A (either A or a) and therefore will not resist as many parasites as the heterozygote.
“The second hypothesis involves avoidance of deleterious recessive genes (also called the general dominance hypothesis), such that heterozygous individuals will express fewer deleterious recessive alleles than its homozygous counterpart.”

Since the MHC region of the genome has a very high degree of variation among individuals, similarity in the MHC region argues for close relationship, and thus for the defective offspring. Use of the present invention for pair matching strongly increases the chances that offspring will be healthy.

For over two centuries in Western cultures, people of childbearing age have been highly mobile and thus often have obscure ancestry. People can thus not always be sure they are not pairing themselves with closely-related partners. The present invention provides a safe, confidential and discreet way of managing this issue.

### Increasing Fertility

The present invention includes a method which selects for more diversity in a couple's children's immune systems, increasing the chance that its children will survive, thrive, and increase the couple's fertility.

The term “fertility” is usually defined as a measure: “fertility rate” is the number of children born per couple, person or population. In this Specification, and in the s that follow, the term “fertility” is used in a longer-term sense, describing the number of a couple's descendants over a few generations compared to that of the population as a whole.

It has been known since antiquity that couples who are closely related have relatively few children who survive until adulthood. The couples often fail to conceive, and their offspring suffer a higher-than-average number of birth defects. As we have shown elsewhere, fetal loss from defects in the embryo, premature delivery and complications of pregnancy are higher for closely-related couples. The fertility (as defined above) of couples who are first or second cousins is poor. First and second cousins had very few grandchildren, while third and fourth cousins had the largest number. In more distant relationships, fertility declined, so that sixth cousins have about the same number of grandchildren as first cousins. Fertility tends to level off at seventh cousins and more distant relationships.

This loss of fertility is not inconsistent with the linear increase in attraction and responsivity noted above. It is important to note that humans and their hominid forebears lived for 3 million years—until about 50,000 years ago—in hunting-gathering camps that contained no more than 50 people; usually about 30. Many times, depending on the culture, men or women would move to a neighboring camp to take a mate. Thus the likelihood of outbreeding beyond fifth or sixth cousin was very low, and there was no evolutionary pressure to limit the degree of outbreeding. A linear increase in attraction and responsivity is completely consistent with those anthropological findings.

### Increasing Fitness

The present invention increases the likelihood of reproductive success. This benefit is accomplished by ensuring that the couple is not, without its knowledge, closely-enough related that their children run a high risk of defects arising from inbreeding, for example those arising from each partner's carrying a recessive deleterious gene.

The term “fitness” is defined as “the probability of reproductive success through one's own offspring.” People who select mates with alleles of genes in the Major Histocompatibility Complex (MHC) that are different from theirs in accordance with the present invention will have more successful pregnancies, offspring with more robust immune systems, and in many cases a greater number of grandchildren. These beneficial consequences comprise the elements of reproductive success.

### Enhancing Immune System Diversity

The present invention enhances the immune system diversity of offspring. One method of the invention selects for more diversity in the immune systems of children, increasing the chance that the children will survive and thrive, and since their children will pass their more-diverse genomes to their own children, thus enhancing their chances of survival and reproduction, the couple's fertility is increased.

Genes in the Major Histocompatibilty Complex (MHC), a region of the short arm of Chromosome 6 in humans, contain information on foreign substances from the environment such as bacteria or viruses causing infectious diseases (antigens) that have been experienced and overcome by individuals and their ancestors. Like most genes, MHC genes contain instructions for cells to manufacture proteins. When an MHC protein is made, mechanisms in the cell clip (ligate) short strands of protein (peptides) from the large protein molecule. These ligands or peptides contain information on the molecular structure of the foreign substances listed above. They migrate to the cell's surface, and inform the immune system of the structure of these legacy substances, and are thus also called antigens (antigen is a general name for a substance that elicits an immune response). The antigens generated by the MHC genes may be called either “histocompatibility antigens” or “human leucocyte antigens.” Cells bearing these antigens on their surfaces are called antigen-presenting cells. That term applies to any of various cells (as a macrophage or a B cell) that take up and process an antigen into a form that, when displayed at the cell surface in combination with a molecule of the Major Histocompatibility Complex, is recognized by and serves to activate a specific Helper T cell. Helper T cells are an important part of the human immune system.

Alleles in the MI-IC genes are codominantly expressed, meaning that if the mother and father carry different alleles (that is, variations) of the same gene, each allele is expressed. The offspring thus carry information on the antigens that have beset both of their ancestral lines. For this reason, if a child's parents' MHC alleles are more diverse (that is, if they share fewer alleles in the MHC region), the offspring have innate immunity to a larger number of diseases.

The present invention's matching method, which selects possible parenting partners on the basis of greater diversity in their MHC alleles, also selects for more diversity in the couple's children's immune systems. This increases the chance that their children will survive and thrive, thus increasing the couple's fertility.

The children not only receive information from infections overcome by their parents' ancestors, but also from those overcome by the parents themselves, since the body has a recently-discovered (and quite complex) mechanism to modify its own genome in response to infections. These modified genes are passed on to those of their offspring who are conceived after the parents have survived the infections.

### Greater Marital Stability

A match predicted by the present invention leads to greater stability of a couple's marriage. Women who are paired with men who have dissimilar alleles in the Major Histocompatibility Complex (MHC) of their genome are not only more strongly attracted to their mates and are more responsive to them, but are also more faithful to them. Men in such pairings are also more faithful to their partners. Men are more likely to be faithful to a partner who not only holds him in high regard, but who is more responsive to him during coitus. See Garver-Apgar, C. E., Gangestad, S. W., Thornhill, R., Miller, R. D., & Olp, J. J., “Major Histocompatibility Complex Alleles, Sexual Responsivity, and Unfaithfulness in Romantic Couples.” Psychological Science, Vol. 17 No. 10, Pages 830-835 (2006).

Pair-bonded women who were near ovulation reported greater extra-pair flirtation and greater mate guarding by their primary partner. As predicted, however, these effects were exhibited primarily by women who perceived their partners to be low on hypothesized good genes indicators (low in sexual attractiveness relative to investment attractiveness). See Haselton, “Conditional expression of women's desires and men's mate guarding across the ovulation cycle,” Hormones and Behavior, Vol. 49, Pages 509-518 (2006).

By analyzing the genomes of offspring of an inbred human population, Ober found strong evidence that there was a greater-than-chance probability that a child's parents had assortative (different) alleles in the MHC region. This implies that couples who had different MHC alleles were responsible for more offspring, whether they were married to each other or not, and further implies that those married couples who had different alleles tended to be more faithful. See Ober, C. Weitkamp, L. R., Cox, N., Dytch, H., Kostyu, D., Elias, S., “HLA and mate choice in humans.” American Journal of Human Genetics, Vol. 61, Pages 497-504 (1997).

Hormonal birth control (“The Pill”) reverses women's preference for complementary MHC alleles. The reason for this is that hormonal birth control (HBC) mimics pregnancy, and that pregnant women prefer to be with their own family, whose MHC alleles are similar to hers. When a woman who is not using hormonal birth control is not pregnant, her unconscious search for the best complement of genes for her children, i.e., a man whose MHC alleles are different from hers, may lead her to be unfaithful to her husband if his alleles are similar to hers.

A couple who meet and marry while the woman is using hormonal birth control is likely to have similar MHC alleles; and thus, it is also more likely that, if for any reason she stops her HBC regimen, she will be less attracted to her husband and more attracted to men with complementary MHC alleles, and thus more likely to stray. Put another way, she will be less attracted to her husband and more likely to stray. The present invention provides a powerful means of counteracting that effect, since it predicts the man's attractiveness to the woman after their marriage has been solemnized, thus leading to a more stable union.

### Mate Assessment

The present invention provides the following benefits:
1. Subscribers to online dating and other dating or introduction services will be able to predict a woman's attraction to a candidate man, thus improving the chances of a compatible match.
2. Individuals will be able to compare their genomes with the goal of entering into satisfying and lasting relationships.
3. Couples considering a long-term relationship or marriage will be able to assess the probable stability of that relationship and the prospective health of their offspring.
4. Because the present invention will reduce birth defects and spontaneous abortions, enormous amounts of public and private money will be saved, and will, at least for a few individuals, provide a much higher quality of life.
5. Research has shown that women who have children fathered by a man with assortative (different) alleles in the MI-IC region have fewer miscarriages and are less likely to experience preeclampsia, a serious complication of pregnancy, and that it is less likely that their children will have birth defects.

In addition to being attracted to men with complementary MHC alleles, heterosexual women who are not using hormonal birth control (HBC) are also sexually more responsive to those men. When women are in physical proximity to men, for example in a social or work setting, they distinguish the degree of difference in their and the man's MHC alleles by scent. Although women are not usually aware of this, numerous studies have proven this beyond reasonable doubt. Surprisingly, in spite of humans' relatively poor sense of smell, people are able to distinguish among MI-IC variations of the same species of mouse by smell alone. The taste and smell of bodily fluids exchanged during kissing also play an important role in mate assessment.

These odors and tastes play an important role in pair-bonding and the maintenance of relationships, as is dramatically illustrated by the pervasive habit of smelling one's partner's clothes in his absence.

For obvious reasons, none of these means of mate assessment is available to people who have never met; and some substitutes that seem quite reasonable, such as viewing still photographs of partnering candidates, actually result in poorer matches than could be achieved by chance.

Poor matches can result even when the prospective partners are in close contact. The use of hormonal contraceptives such as birth-control pills reverses usual female preferences for male scent, increasing the chances that a union would result in birth defects, pregnancy complications, such as miscarriages and spontaneous abortions, and marital infidelity.

These benefits are limited to heterosexual individuals. People of other sexual orientations have different odor preferences. The relationship prediction methods of the present invention can also be used to assist these prospective couples in finding compatible mates.

### Reducing Miscarriages

The present invention reduces the likelihood that a woman will suffer a miscarriage. One embodiment of the invention ensures that a couple is not, without its knowledge, closely-enough related that its children run a high risk of defects arising from inbreeding, for example those arising from each partner's carrying a recessive deleterious gene. Couples without “chemistry” are twice as likely to miscarry.

There is a considerable body of research pointing to the relationship of miscarriages (spontaneous abortions) and preterm births (premature babies) to parents who have similar alleles (that is, variations) of genes in the Major Histocompatibilty Complex (MHC), a region of the short arm of Chromosome 6 in humans. The antigens generated by the MHC genes are called both histocompatibility antigens and human leucocyte antigens (HLA). In a survey of the field published in 1999, Ober found that “Increased fetal loss rates among couples matching for HLA-B or for the entire haplotype suggest that compatible fetuses are less likely to survive to term than incompatible fetuses.” See Ober, Carole, “Studies of HLA, fertility and mate choice in a human isolate,” Human Reproductive Update 1999 (Publication of the European Society of Human Reproduction and Embryology), Vol. 5, No. 2 Pages 103-107 (1999). Elsewhere in the cited paper, she notes that Komlos and Schacter show “evidence demonstrating increased HLA sharing among couples with recurrent spontaneous abortion (RSA) compared with control couples . . . .” Other work by Ober provides an enormous volume of data supporting the relationship of fetal loss to similarity in MHC alleles. See Komlos, L., Zamir, R., Joshua, H., and Halbrecht, I., “Common HLA Antigens in Couples with Repeated Abortions,” Clinical Immunology and Immunopathology 7, Pages 330-335 (1977). See Schacter, B., Muir, A., Gyves, M. et al., “HLA-A, B compatibility in parents of offspring with neural-tube defects or couples experiencing involuntary fetal wastage,” The Lancet, Apr. 14, 1979, Pages 796-799.

Differing alleles in the HLA-G gene in the MHC region may decrease the chance of spontaneous abortions and preeclampsia, a complication of pregnancy which endangers both the mother and her fetus.

Preterm births levy an enormous cost on society. The Institute of Medicine (part of the National Academy of Sciences, estimates that, preterm births in the U.S. cost at least $26.2 billion in 2005, or an average of $51,600 per infant.

Women tend to select mates with differing alleles in the MHC region of their genome. The method of the present invention will substantially reduce fetal loss in couples.

### Reducing Preeclampsia

The present invention reduces the chance that a woman will suffer preeclampsia in her pregnancy. There is a higher risk of preeclampsia in couples with similar alleles in the MHC region of their genome. There are two mechanisms for this effect:
1. Women who carry a polymorphic allele of the HLA-G gene, which is expressed by the fetus and influences its placenta's attachment to the uterus) are at a higher risk of preeclampsia and fetal loss. The presence of the usual (monomorphic) form of the gene in the father's genome halves the chance that the fetus will inherit (and express) the variant polymorphic form.
2. The incidence of preeclampsia is related to the mother's tolerance to the father's genetic material. This tolerance increases through continued physical contact.

Since marital fidelity and pair bonding are higher between partners who have differing alleles in the MHC region of their genome, the present invention's use of genetic matching to increase the chances of diversity in the MHC regions of the couple's genomes will reduce the chance of preeclampsia in the mother and its consequent risk to her and her unborn child.

Hormonal birth control, e.g., the Pill, reverses women's preference for men with complementary MHC alleles. The reason for this is that the hormones used in hormonal birth control (HBC) are similar to those present in a woman's body during pregnancy, and their effect therefore mimics pregnancy; and that a pregnant woman prefers to be with her own family, whose MHC alleles are similar to hers. When a woman who is not using hormonal birth control is not pregnant, her quest for a good father for her children—a man whose MHC alleles are different from hers—may lead her to be unfaithful to her husband if his alleles are similar to hers.

A couple who meet and many while the woman is using hormonal birth control is likely to have similar MHC alleles; and thus, it is also more likely that, if for any reason she stops her HBC regimen, she will be less attracted to her husband and more attracted to men with complementary MHC alleles, and thus more likely to stray.

As discussed elsewhere in this application, if she conceives with her husband and her husband has similar MI-IC alleles, this may also lead to difficulties in pregnancy, unwanted miscarriages, poor fertility and impaired immunity in the couple's children. The present invention provides a powerful means of counteracting that effect, since it predicts the man's attractiveness to the woman after their marriage has been solemnized, thus leading to a more stable union.

Women who are presented with an array of still photographs of men and are asked to select men with whom they would consider having a relationship tend to select men with similar, not different, MHC alleles. In cultures where arranged marriages are common and in situations in which a matchmaker or other gobetween is involved, and the woman, having selected a man from such a photographic array, is under great pressure to proceed with the relationship, the chances of the woman's not being attracted to the man, and thus having an unsatisfactory relationship and the other adverse effects discussed above, is high. See Roberts, S. Craig, et al., “MHC-assortative facial preferences in humans,” Biology Letters, Vol. 1, Pages 400-403 (2005).

In cultures with arranged marriages or in those where matchmakers are used, the bride-to-be is usually quite young and has had little contact with men outside her family. She is therefore not in a position to select among candidates based upon the natural means (scent) at her disposal.

It is therefore of considerable value to dating services, matchmakers, parents in societies in which arranged marriages are common, and to the prospective partners themselves, to be able to predict the woman's attraction to a particular man during the fertile part of a long-term relationship, when for obvious reasons HBC is not used.

### VIII. Responsivity

One embodiment of the present invention may be used to predict a good relationship. This prediction may be determined, in whole or in part, upon a woman's responsivity to a prospective male match. In this Specification, and in the s that follow, the term “responsivity” is defined as:
Sexual responsivity refers to the extent to which women are willing, interested, and enthusiastic about having sex with a romantic partner, the degree to which they are interested in trying to please a romantic partner sexually, and the degree to which they are sexually “turned on” and satisfied by a romantic partner.

### IX. Alternative Method: When a Woman is Using Hormonal Birth Control

Hormonal birth control (“The Pill”) reverses women's preference for men with complementary MHC alleles. The reason for this is that the hormones used in hormonal birth control (HBC) are similar to those present in a woman's body during pregnancy, and their effect therefore mimics pregnancy; and that a pregnant woman prefers to be with her own family, whose MHC alleles are similar to hers. When a woman who is not using hormonal birth control is not pregnant, her quest for a good father for her children—a man whose MHC alleles are different from hers—may lead her to be unfaithful to her husband if his alleles are similar to hers.

If a woman who uses the present invention to obtain a relationship prediction uses hormonal birth control, she may be provided with a report or instructions which may help her make a better-informed decision. So, for example, a relationship prediction for a woman using hormonal birth control may be, generated based on the woman's preference for a man with complementary MHC alleles.

### X. Hormonal Birth Control & Attractiveness

One embodiment of the present invention may be used to predict an enduring relationship between a man and a woman. In this embodiment, a woman is advised that a man may find her less attractive if she changes her hormonal birth control regimen. The man's diminished attraction to the woman results from a change caused when the woman starts or stops using hormonal birth control.

In another embodiment, a website is operated which enables customers to access information presented on the website. The customer may request advice concerning the maintenance of a good relationship. Advice is provided to the customer in response to a request conveyed to the website. In one embodiment of the invention, this advice may include a recommendation that a man may find a woman less attractive if she changes her hormonal birth control regimen. In an alternative embodiment, the customer's request and the advice furnished in response may be conveyed in person, at a doctor's office or clinic, over the telephone, or by some other suitable means.

In yet another embodiment, the website may be used to ask a female if she has recently started or stopped using hormonal birth control, and if she believes that her male mate finds her less attractive since this change in her use of hormonal birth control. These responses are then correlated, and relationship advice is furnished to others based on the correlated data. This relationship advice may be supplied free of charge as a public service.

In this Specification and in the Claims that follow, the terms “hormonal birth control” is intended to include all hormonal contraceptives that contain progestin or one of its analogues and/or estrogen or one of its analogues. They include birth-control pills, certain intra-uterine devices (e.g., Mirena), vaginal rings, Norplant implants, contraceptive injections and their ilk.

### XI. Custom-Fabricated Perfumes

FIG. 66 illustrates the step 200 of a customer 17b receiving a custom-formulated perfume 202, “MyAroma™” or “MyCologne™,” which contains olfactory reagents that correspond to her genetic attributes, and specifically, which correspond to his or her MI-IC-derived peptide profile.

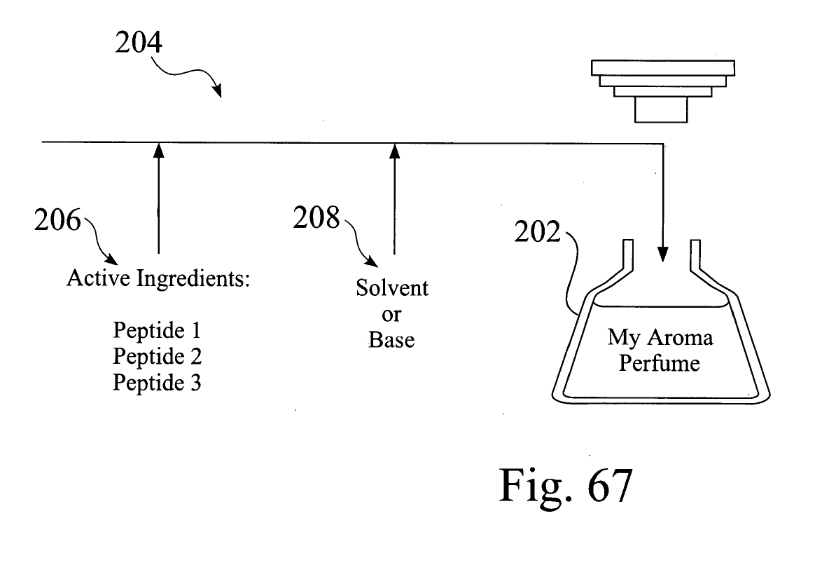

FIG. 67 depicts a method 204 of manufacturing a customized perfume 202. General methods for manufacturing compositions for dispensing fragrances, aromas and perfumes are well known in the art. According to the Scented Products Education and Information Association of Canada, ingredients in a typical fragrance “recipe” generally include:
“extracts from plants and flowers (naturals),
synthetic recreations (synthetic duplications of natural fragrance materials),
synthetic innovations (variations of naturally-occurring materials which have unique olfactory properties).
In general, typical fragrance formulae contain 100-350 ingredients, with an average concentration of usually less than 1%.
“In a perfume, ethyl alcohol (of the same grade and purity as in alcoholic beverages) composes 50-90% of the product, purified water may constitute 5-20% of the product, with the fragrance component accounting from 10-30% of the finished product. Also present are UV inhibitors (to prevent degradation in the bottle) and any additional colouring agents.”

### SPEIAC, 20 Britannia Road East, Suite 102, Mississauga, Ontario L4Z 3L5.

In one embodiment of the present invention, appropriate combinations of biological, synthetic or other agents such as peptides or other substances are added as active ingredients 206 to a base 208 to a mixture, together with and/or any other suitable solvents, stabilizers, agents, preservatives, dispersants, inhibitors or components. In one embodiment, the base is a solvent, such as alcohol or water. These biological agents are selected to match a genetic attribute possessed by a person.

In one implementation, the perfume or cologne 202 made in accordance with the invention contains substances which are complementary to the user's Major Histocompatibility Complex (MHC profile), which will be attractive to the same user. In the same implementation, that person may ask a spouse or mate to wear this perfume or cologne 202, which pleases the person for whom the customized perfume or cologne was made. The present invention includes both perfume or cologne intended to be used by a person selecting the perfume or cologne for herself or himself, as well as an “inverse perfume or cologne,” which is selected by one person and used by another.

The biological agents may be selected to promote the responsivity of the person using the mixture, or may be selected to promote the responsivity of another person using the mixture. The biological agents in the mixture may be used to broadcast or indicate sexual compatibility, interest, awareness or attraction. As an alternative, the biological agents may be selected to promote confidence, self-esteem or the interest or attraction of another. The invention may be used to promote relationships between members of the opposite sex, or between members of the same sex.

The specific composition of the mixture may take many forms, including, but not limited to a perfume, a cologne, a salve, a paste, an aerosol spray, a powder, or a cosmetic. The cosmetic may include skin cream, lipstick, lip balm, gel, ointment, colorant, or some other preparation that be applied to the body. The mixture is generally intended to be applied to, dispensed on or worn on the skin or hair, but may be applied on or used in conjunction with an article of clothing, which may be impregnated with the active ingredients. In yet another embodiment, the perfume 202 may be encapsulated or contained in a pill or medication that is taken internally, and which is then secreted through the skin or which causes a biological reaction which produces or mimics an odor. The mixture may also be dispensed using a variety of devices, including, but not limited to air fresheners, aroma-dispensing devices, candles and incense.

This specialized perfume 202 contains a strong preparation of personal peptides, enabling the user to “broadcast” his or her “MHC” over a wide area, and increasing his or her chances of meeting a compatible partner.

The MHC is a cluster of genes that determines details of cellular surfaces and thus immune responses, and specifies certain peptides that appear in skin secretions and urine. These peptides are responsible for odors which uniquely identify individuals who are not identical twins. Detailed information concerning the MHC may be found in Leslie A. Knapp's publication entitled The ABCs of MHC, published in Evolutionary Anthropology 14:28-37 (2005) Wiley-InterScience. MyAroma™, MyPerfume™, MyEssence™ are Trade & Service Marks owned by the Assignee of the Present patent application.

### XII. A Graphical Aid for Interpreting Test Results

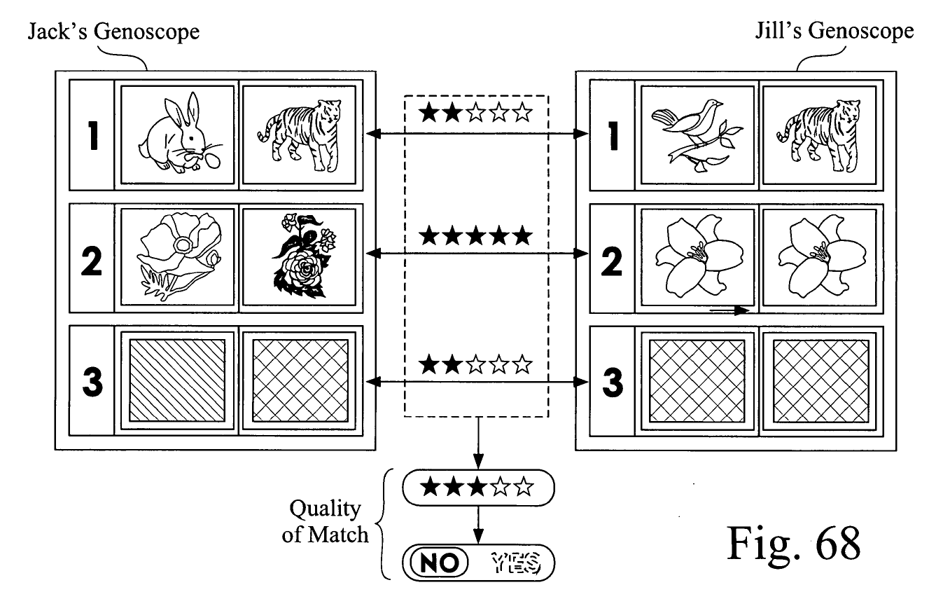

FIG. 68 presents one particular version of a graphic representation or “Genoscope™,” which illustrates a hypothetical portion of test results for a customer, and which enables the customer to easily understand the quality of a match with another person.

The same prediction method described elsewhere in this application is useful in the absence of a dating service, or in situations where a person prefers to make their own initial contacts with prospective mates or in cases in which a person wishes to assess a potential mate from a field of known candidates. In one embodiment, a person (hereafter the “User”) submits his or her tissue, fluid or other biological sample to a laboratory for typing, and the laboratory provides the User with an alphanumeric code (the “Code”) which describes his or her genetic information, i.e., genome in the MHC region and any other regions of interest. The User may then compare his or her Code with that of another User to estimate their mutual compatibility and thus the quality of a romantic relationship that might ensue. Each User would be provided with a written guide or computer program for comparing the data embedded in the Codes and estimating the quality of the contemplated relationship.

In another embodiment, the laboratory would keep records of the genomes or Codes of each User. On request, the laboratory would compare the User's genomes or Codes for compatibility and issue a report.

An example of such a report, in easily-understood graphical form, is shown in FIG. 68. A variety of symbols are presented in a grid along rows and columns. The more symbols match, the more genomes are similar, which predict a bad match. The stars in the center of FIG. 68 indicate the approximate strength or quality of a match, with zero being the lowest quality, and five stars representing the highest. An added feature of the report could be a Compatibility Score which rates the predicted quality of the contemplated relationship in a way that can be compared with other relationships, for example on a scale of one to ten, with ten being the best possible genetic compatibility score.

### GLOSSARY

### Allele:

Either of a pair of alternative Mendelian characters (as smooth or wrinkled seed in the pea) (Webster).

### Antigen:

1. A usually protein or carbohydrate substance (as a toxin, enzyme, or any of certain constituents of blood corpuscles or of other cells), that when introduced into the body stimulates the production of an antibody;
2. A substance that reacts in complement fixation with an antibody to bind complement, the antigen and antibody usually being specific (Webster).
Antigen-presenting cell:
Any of various cells (as a macrophage or a B cell) that take up and process an antigen into a form that when displayed at the cell surface in combination with a molecule of the major histocompatibility complex is recognized by and serves to activate a specific Helper T cell.

### Attractive:

Having qualities that arouse interest, pleasure, or affection in the observer. attractiveness.

### Attribute:

A quality, character, or characteristic ascribed usually commonly: a: a characteristic either essential and intrinsic or accidental and concomitant b: a quality intrinsic, inherent, naturally belonging to a thing or person (Webster).
Attribute (genetic):
An attribute as defined above which is controlled or caused by a creature's genome.
Body odor:
The characteristic odor of a living animal body.

### Chromosome:

One of the more or less rodlike chromatin-containing basophilic bodies constituting the genome and chiefly detectable in the mitotic or meiotic nucleus that are regarded as the seat of the genes, consist of one or more intimately associated chromatids functioning as a unit, and are relatively constant in number in the cells of any one kind of plant or animal (Webster).
Codominant genes:
A set of two or more alleles, each expressed phenotypically in the presence of the other (Online Medical Dictionary).

### Consanguinity:

The quality or state of being related by blood or descended from a common ancestor (Webster).
Diversity in the MHC regions:
The degree to which the alleles in the Major Histocompatibility Complex differ between two individual members of the same species.
Dominant gene:
A gene that is expressed phenotypically in heterozygous or homozygous individuals.

### Fertility:

Actual reproductive capacity as measured by production of offspring (Webster). In the context of this application, this includes descendants more distant than direct offspring.

### Fitness:

The fitness of the individual—having an array x of phenotypes—is the probability, s(x), that the individual will be included among the group selected as parents of the next generation.” See Hartl, D. L. A Primer of Population Genetics. Sinauer, Sunderland, Mass., 1981 (Wikipedia).

### Gene:

One of the elements of the germ plasm serving as specific transmitters of hereditary characters and usually regarded as portions of deoxyribonucleic acids linearly arranged in fixed positions and as functioning through control of the synthesis of specific polypeptide chains.
Group frequency:
The frequency of occurrence of a particular group of genes or alleles in a population.

### Haplotype:

A combination of alleles at multiple loci that are transmitted together on the same chromosome. Haplotype may refer to as few as two loci or to an entire chromosome depending on the number of recombination events that have occurred between a given set of loci (Wikipedia).

### HBC:

See hormonal birth control.
HBC regimen:
The process of maintaining an effective level of birth-control hormones in one's body.

### Heterozygote:

A cell formed by the union of heterozygous gametes: a fertilized egg: broadly: the developing individual produced from such a cell.

### Heterozygous:

Producing two types of gametes with respect to one or more allelomorphic characters.
Histocompatibility antigen:
Any of the antigenic glycoproteins on the surface membranes of cells that enable the body's immune system to recognize a cell as native or foreign and that are determined by the major histocompatibility complex.

### HLA:

### See Human Leucocyte Antigen.

### Homozygote:

A cell formed by the union of homozygous gametes: a fertilized egg: broadly: the developing individual produced from such a cell.
Homozygous: possessing genes for only one member of at least one pair of allelomorphic characters.
Hormonal birth control (HBC):
The use of drugs containing progestin (or one of its analogues) and/or estrogen (or one of its analogues) or any other natural or synthetic hormone to control ovulation, implantation or conception and thus prevent unwanted pregnancy. These drugs may be administered orally, parentarilly or by any other means.
Human leukocyte antigen:
Any of various proteins that are encoded by genes of the major histocompatibility complex in humans and are found on the surface of many cell types (as white blood cells).
Infectious disease:
A disease caused by the entrance into and growth and multiplication in the body of bacteria, protozoans, fungi, or analogous organisms (such as filterable viruses).
Immune system diversity:
The ability of a creature's immune system to recognize a variety of threats.
Locus, plural loci:
A fixed position on a chromosome such as the position of a biomarker that may be occupied by one or more genes.

### Major Histocompatibility Complex:

A group of genes that function especially in determining the histocompatibility antigens found on cell surfaces and that in man comprise the alleles occurring at four loci on the short arm of chromosome 6—abbreviation MHC.
Marital stability:
The degree to which a marriage persists.

### MHC: See Major Histocompatibility Complex.

Ovulation cycle:
In a mammal, the periodic release of eggs into the uterus. More generally, the cycle which includes menstruation, ovulation, and in most mammals, estrus.

### Peptide:

Any of a class of amides that are derived from two or more amino acids by combination of the amino group of one acid with the carboxyl group of another, that yield these acids on hydrolysis, that are classified according to the number of component amino acids, and that are obtained by partial hydrolysis of proteins or by synthesis (as from alpha-amino acids or their derivatives). A chain of amino acids produced by a living cell.

### Perfume:

A substance that emits an odor.

### Preeclampsia:

A toxic condition developing in late pregnancy characterized by a sudden rise in blood pressure, excessive gain in weight, generalized edema, albuminuria, severe headache, and visual disturbances (Webster's Unabridged).

### Prediction:

An inference regarding a future event based on probability theory Webster's Unabridged).

### Polymorphic:

Having or occurring in several distinct forms: exhibiting polymorphism.
“Polymorphic” refers to the rare occurrence of a variant of the usually-monomorphic HLA-G gene.
Population dataset:
A set of data derived from statistics from a study of population of humans.
Recessive gene:
A gene that is phenotypically expressed in the homozygous state but has its expression masked in the presence of a dominant gene.

### Relationship:

The state of affairs existing between two people or among two or more people.

### Responsivity:

The extent to which women are willing, interested, and enthusiastic about having sex with a romantic partner, the degree to which they are interested in trying to please a romantic partner sexually, and the degree to which they are sexually “turned on” and satisfied by a romantic partner.
Citations marked “Webster” are from Webster's Third New International Dictionary, Unabridged. Merriam-Webster, 2002.

### CONCLUSION

Although the present invention has been described in detail with reference to one or more preferred embodiments, persons possessing ordinary skill in the art to which this invention pertains will appreciate that various modifications and enhancements may be made without departing from the spirit and scope of the Claims that follow. The various alternatives for providing Searching Methods Using Genetic Responsivity Measurements that have been disclosed above are intended to educate the reader about preferred embodiments of the invention, and are not intended to constrain the limits of the invention or the scope of the Claims.

### LIST OF REFERENCE CHARACTERS

10 MateFinder™
10a First user's MateFinder
10b Second user's MateFinder
11 Interrogation or seeking signal
11a First interrogation signal
11b Second interrogation signal
12 Housing
14 Power switch
15 “Seeking” indicator light
16 “Match Found” indicator light
16a First match indicator
16b Second match indicator
17a Man
17b Woman
18 LCD message screen
19 Website
20 USB port
22 Personal computer
24 USB cable
26 Battery
28 Radio/Processor assembly
30 Antenna
32 Memory
32a First memory
32b Second memory
33 Attribute
33a First set of attributes
33b Second set of attributes
34 Mask switch
35 Correlation thumbwheel
36 Microprocessor
37 Wireless network
38 Receiver
39 Transmit/Receive Switch
44 Transmitter
48 Bandpass filter
49 MateFinder with Cellular Telephone Combination
54 Attribute
56 Target
58 Relevance Difference Measurement
111 Personal computer
112 Internet Dating Service Website
113 Web page for opening new account
114 Web page for placing order
115 Test
117 Telephone
118 Retail store
119A Physician or health care provider
119B Religious leader or cleric
120 Bottle of cleaning solution
122 Cotton ball
124 Sample patch
124C Central area of patch
124S Strips extending away from central area
125 Plaster
126 Antibiotic
127 Adhesive
128 Sealable plastic bag
130 Mailing envelope, pouch or box
132 Lab technician
134 Sample analyzer
135 Analysis results
136 Good matches suggested to customer on website
137 Test results received from physician
138 Postal worker
140 Tissue sample obtained from cheek swab
142 Shopping mall
144 Kiosk
146 Odor sample captured from air surrounding person
148 Collect sample
150 Open saliva collection cup
152 Screw cap on cup and mix sample
154 Cap
156 Place closed cap in bag and seal
158 Place bag in mailer
160 Seal mailing box
162 Mail box containing saliva sample to laboratory
164 Laboratory collection kit preparation tasks
166 Internet Dating Service tasks
168 Customer tasks
170 Laboratory analysis, matching and reporting tasks
172 Dating Service and laboratory cooperative tasks
174 Graph of MHC alleles shared on the horizontal axis, a female sexual responsivity to partner on the vertical axis
176 Bar chart showing the number of MHC alleles shared on the horizontal axis, and the expected female sexual responsivity to partner on the vertical axis
178 Chart that shows the relationship of alleles in the MHC Group on Human Chromosome No. 6
180 WIC Allele Groups
182 HLA-A Allele Group Frequency for a European Population Dataset
184 HLA-B Allele Group Frequency for a European Population Dataset
186 HLA-DR [symbol] 1 Allele Group Frequency for a European Population Dataset
188 Allele Group Frequencies
190 A/B/DR [symbol] 1 Group Haplotype Frequency
194 Man using a MateFinder™ device
196 MateFinder™ device
198 Detailed view of a MateFinder™ device
200 Woman whose tissue sample has already been analyzed receives a custom-formulated perfume which contains aromas that correspond to her genetic attributes
202 Custom perfume based on genetic attributes
204 Method of manufacturing a customized perfume
206 Active ingredients
208 Solvent or base

### List of Variables Used in Mathematical Expressions

### Symbols in Mathematical Expressions From the First Group of Embodiments:

d Difference Measurement
(x₁, y₁) coordinates of a first point in the plane
(x₂, y₂) coordinates of a second point in the plane
n or k an index which identifies an attribute, a weight, or an information source
N the number of attributes or weights under consideration (Expressions 1-10)
F Fred
M Mary
Fₙ The value of Fred's n^{th} attribute
Mₙ The value of Mary's n^{th} attribute
wₙ Relative weight for an nth attribute (sum of all such weights is 1)
Wₙ Weight for an n^{th} attribute before converting to a relative weight
FD The set of attributes which Fred desires in a mate
FDₙ The n^{th} attribute in the set of attributes Fred desires in a mate
FE The set of attributes which Fred possesses
MD The set of attributes which Mary desires in a mate
ME The set of attributes which Mary possesses
MEₙ The n^{th} attribute in the set of attributes which Mary possesses
d_{BDE} Bidirectional Difference Measurement, desired-to-existing attributes
ε Symbol for “is contained in”
A−R The set of attributes that are in set A but not in set R
T Threshold for deciding if there is a match
a, b Lower and upper limits for the range of an attribute value
x, y The original and converted attribute values in Expression 9
c Correlation between two sets of attributes

### Symbols in Mathematical Expressions From the Third Group of Embodiments:

M The number of attributes under consideration in a search
N The number of sources found in a search
m An index which identifies a search attribute
n An index which identifies a source found in a search
fₘₙ The number of occurrences of the attribute Am in the source Sₙ
Rₙ Relevance value for source Sₙ
Wₙ Weight used in calculating Rₙ
Dₙ Difference Measurement from nth source to the set of search attributes

### Symbols in Mathematical Expressions From the Fourth Embodiment:

N The number of elements (components) within a customer's vector
k An index which identifies an element (component) within a customer's vector
aₖ and bₖ The values of the k^{th} element (component) of vector a for one customer and vector b for the other customer
β An exponent (power) used in Expression (17), which is a positive real number
Wₖ A weight which can be used with Expressions (14)-(20), as exemplified in Expression (21)
a^{T} The transpose of vector a
W A weighting matrix used in Expression (22)

What is claimed is:

1. An apparatus, comprising:
a first transceiver (10a);
said first transceiver (10a) including a first memory (32a); said first memory (32a) for storing a first attribute (33a) selected by a first user (17a);
said first transceiver (10a) including a first match indicator (16a);
a second transceiver (10b);
said second transceiver (10b) including a second memory (32b); said second memory (32b) for storing a second attribute (33b) selected by a second user (17b);
said second transceiver (10b) including a second match indicator (16b);
said first transceiver (10a) for emitting a first interrogation signal (11a);
said first interrogation signal (11a) being received by said second transceiver (10b);
said first match indicator (16a) on said first transceiver (10a) being activated when said first interrogation signal (11a) finds a match between said first attribute set (33a) stored in said first memory (32a) in said first transceiver (10a) and said second attribute set (33b) stored in said second memory (32b) in said second transceiver (10b);
a wireless network (37);
a first network radio (28a); said first network radio (28a) being co-located with said first transceiver (10a); said first network radio (28a) being used to communicate over said wireless network (37); and
said match being determined using a Difference Measurement formula.

2. An apparatus as recited in claim 1, in which said Genetic Responsivity Measurement formula is used to correlate said first attribute set (33a) and said second attribute set (33b).

3. An apparatus as recited in claim 1, in which said Genetic Responsivity Measurement formula is stored in said first and said second memory (32a, 32b).

4. An apparatus as recited in claim 1, in which said Genetic Responsivity Measurement formula is incorporated in software that runs on a server that enables the operation of an Internet dating website.

5. An apparatus as recited in claim 1, in which a correlation between said first and said second attribute sets (33a, 33b) is computed using the expression
$$d ( F , M ) = \sqrt{{\sum }_{n = 1}^{N} {( {F}_{n} - {M}_{n} )}^{2}}$$
where an individual possessing the first set of attributes is identified as F;
an individual possessing the second set of attributes (x₂, y₂) is identified as M;
d(F, M) is a Genetic Responsivity Measurement between F and M; N is a number of attributes;
F₁, F₂, . . . , F_{N} represent a plurality of attribute values for F; and
M₁, M₂, . . . , M_{N} represent a plurality of attribute values for M.

6. An apparatus as recited in claim 1, in which a correlation between said first and said second attribute (33a, 33b) is computed using the expression
$$d ( F , M ) = {\sum }_{n = 1}^{N} {w}_{n} \vert {F}_{n} - {M}_{n} \vert$$
where ∥ denotes absolute value;
wₙ is a number between 0 and 1 which assigns a weight to an n^{th} attribute;
Fₙ is the nth attribute of a first individual; and
Mₙ is the nth attribute of a second individual.

7. An apparatus as recited in claim 6, in which a value for a relative weight is computed using the expression:
$${w}_{n} = \frac{{W}_{n}}{{\sum }_{n = 1}^{N} {W}_{k}}$$
where Wₙ is an initially chosen weight for the n^{th} attribute; and
Wₖ is an initially chosen weight for the k^{th} attribute.

8. An apparatus as recited in claim 1, in which a Genetic Responsivity Measurement from a first desired attribute set to a second existing attribute set is computed using the expression:
$$d ( FD , ME ) = {\sum }_{n = 1}^{N} {w}_{n} \vert {FD}_{n} - {ME}_{n} \vert$$
where FD is a desired attribute set of a first individual;
ME is an existing attribute set of a second individual; and
wₙ, is a relative weight for the n^{th} attribute.

9. An apparatus as recited in claim 1, in which a bidirectional match is computed using the expression
$${d}_{BDE} ( F , M ) = \frac{d ( FD , ME ) + d ( MD , FE )}{2}$$
where FD is a desired attribute of a first individual;
ME is an existing attribute of a second individual;
MD is a desired attribute of said second individual;
FE is an existing attribute of said first individual; and
d is a Genetic Responsivity Measurement between the attributes in parentheses.

10. An apparatus as recited in claim 1, in which a relative weight wₙ for all attributes is computed using the expression:
$${w}_{n} = \{ \begin{matrix}1 & if n ∈ R \\ \frac{{W}_{n}}{{\sum }_{k ∈ A - R} {W}_{k}} & if n ∈ A - R\end{matrix}$$
where ε means “is contained in”;
A−R denotes a set of attributes that are in A but not in R; and
Wₖ is an initially chosen weight for the k^{th} attribute.

11. An apparatus as recited in claim 1, in which said Genetic Responsivity Measurement is converted to a correlation using the expression
c(F,M)=1−d(F,M)
where c is a correlation value; and
d is a Genetic Responsivity Measurement between F and M.

12. A method, comprising the steps of:
providing a device (10); said device (10) including a memory (32) and a radio (28);
storing a Genetic Responsivity Measurement formula in said memory (32);
storing a plurality of attributes in said memory (32);
said Genetic Responsivity Measurement formula being used to measure the dissimilarity in a pair of said plurality of attributes; and
using said device (10) to find a good match based on a correlation produced by said Genetic Responsivity Measurement formula.

13. A method as recited in claim 12, in which said Genetic Responsivity Measurement formula is used to correlate said first attribute (33a) and said second attribute (33b).

14. A method as recited in claim 12, in which said Genetic Responsivity Measurement formula is stored in said first and said second memory (32a, 32b).

15. A method as recited in claim 12, in which said Genetic Responsivity Measurement formula is incorporated in software that runs on a server that enables the operation of an Internet dating website.

16. A method as recited in claim 12, in which a correlation between said first and said second attribute sets (33a, 33b) is computed using the expression
$$d ( F , M ) = \sqrt{{\sum }_{n = 1}^{N} {( {F}_{n} - {M}_{n} )}^{2}}$$
where an individual possessing the first set of attributes (x₂, y₂) is identified as M;
an individual possessing the second set of attributes (x₂, y₂) is identified as M;
d(F,M) is a Genetic Responsivity Measurement between F and M; N is a number of attributes;
F₁, F₂, . . . , F_{N} represent a plurality of attribute values for F; and
M₁, M₂, . . . , M_{N} represent a plurality of attribute values for M.

17. A method as recited in claim 12, in which a correlation between said first and said second attribute sets (33a, 33b) is computed using the expression
$$d ( F , M ) = {\sum }_{n = 1}^{N} {w}_{n} \vert {F}_{n} - {M}_{n} \vert$$
where ∥ denotes absolute value; and
wₙ is a number between 0 and 1 which assigns a weight to an n^{th} attribute.

18. A method as recited in claim 17, in which a value for a relative weight is computed using the expression:
$${w}_{n} = \frac{{W}_{n}}{{\sum }_{k = 1}^{N} {W}_{k}}$$
where Wₙ is an initially chosen weight for the n^{th} attribute.

19. A method as recited in claim 12, in which a Genetic Responsivity Measurement from a first desired attribute set to a second existing attribute set is computed using the expression:
$$d ( FD , ME ) = {\sum }_{n = 1}^{N} {w}_{n} \vert {FD}_{n} - {ME}_{n} \vert$$
where FD is a desired attribute of a first individual; and
ME is an existing attribute of a second individual.

20. A method as recited in claim 12, in which a bidirectional match is computed using the expression
$${d}_{BDE} ( F , M ) = \frac{d ( FD , ME ) + d ( MD , FE )}{2}$$
where FD is a desired attribute of a first individual;
ME is an existing attribute of a second individual;
MD is a desired attribute of said second individual; and
FE is an existing attribute of said first individual.

21. A method as recited in claim 12, in which a relative weight wₙ for all attributes is computed using the expression:
$${w}_{n} = \{ \begin{matrix}1 & if n ∈ R \\ \frac{{W}_{n}}{{\sum }_{k ∈ A - R} {W}_{k}} & if n ∈ A - R\end{matrix}$$
Where ε means “is contained in”; and
A−R denotes a set of attributes that are in A but not in R.

22. A method as recited in claim 12, in which said Genetic Responsivity Measurement is converted to a correlation using the expression
c(F,M)=1−d(F,M)

23. A method, comprising the steps of:
accessing a master set (MS); said master set (MS) including a plurality of sources of information (S1, S2, S3, S4, S5, S6, S7, . . . S{N});
generating a set of attributes (54); said set of attributes (54) including a plurality of individual attributes (54A, 54B, 54C . . . 54D) and collectively describing a target (56);
providing a Genetic Responsivity Measurement formula (Expression 1);
computing a set of relevance Genetic Responsivity Measurements (58); said set of relevance Genetic Responsivity Measurements (58) including a plurality of individual Genetic Responsivity Measurements (58A, 58B, 58C, 58D) between each of said individual attributes (54A, 54B, 54C . . . 54D) and said plurality of sources of information (S1, S2, S3, S4, S5, S6, S7, . . . S{N});
selecting a subset of sources of information (S1′, S2′, S3′); and
ranking said subset of sources of information (S1′, S2′, S3′) in the general order of shortest relevance Genetic Responsivity Measurements (58) measured between said subset of sources of information and said set of attributes (54).

24. A method as recited in claim 23, further comprising the step of:
performing a page rank search plurality of individual attributes (54A, 54B, 54C . . . 54D) to reduce the total number of sources of information (S1, S2, S3, S4, S5, S6, S7, . . . S{N}) prior to using said Genetic Responsivity Measurement formula (Expression 1) to measure said relevance Genetic Responsivity Measurements (58).

25. A method as recited in claim 23, in which said plurality of sources of information includes a web page.

26. A method as recited in claim 23, in which said plurality of sources of information includes a record.

27. A method as recited in claim 23, in which said plurality of sources of information includes a document.

28. A method as recited in claim 23, in which said plurality of sources of information includes an address of an RFID device.

29. A method as recited in claim 23, in which the method step of ranking said subset of sources of information (S1', S2′, S3′) in the general order of shortest relevance Genetic Responsivity Measurements (58) measured between said subset of sources of information and said set of attributes (54) is used to create a website for finding generic products.

30. A method as recited in claim 23, in which the method step of ranking said subset of sources of information (S1′, S2′, S3′) in the general order of shortest relevance Genetic Responsivity Measurements (58) measured between said subset of sources of information and said set of attributes (54) is used to create a website for finding a replacement part.

31. A method of searching for a match, comprising the steps of:
providing a first set (F,MS) of data identifiers (Fₙ,SI);
generating a second set of user-selected data identifiers (Mₙ,54) relating to a target (M,56);
computing a set of relevance Genetic Responsivity Measurements (58) comprising a plurality of individual Genetic Responsivity Measurements (58A-D) between respective data identifiers of the first and second sets;
providing a Genetic Responsivity Measurement formula which is a function which combines the plurality of individual Genetic Responsivity Measurements; and
applying the formula to determine an overall degree of match.

32. A method according to claim 31, wherein weighting factors Wₙ are assigned to the identifiers.

33. A method according to claim 31, wherein the data identifiers (Fₙ) of the first set relate to a first entity (F) and the data identifiers (Mₙ) of the second set relate to a second entity (M), whereby the formula determines an overall degree of match between the entities.

34. A method according to claim 33, wherein the Genetic Responsivity Measurement formula provided is:
$$d ( F , M ) = \sqrt{{\sum }_{n = 1}^{N} {( {F}_{n} - {M}_{n} )}^{2}}$$
where an entity possessing the first set of attributes is identified as F;
an entity possessing the second set of attributes is identified as M;
d(F,M) is a Genetic Responsivity Measurement between F and M;
N is a number of attributes;
F₁, F₂, . . . , F_{N} represent a plurality of attribute values for F; and
M₁, M₂, . . . , M_{N} represent a plurality of attribute values for M.

35. A method according to claim 34, wherein the Genetic Responsivity Measurement formula provided is:
$$d ( F , M ) = {\sum }_{n = 1}^{N} {w}_{n} \vert {F}_{n} - {M}_{n} \vert$$
where an entity possessing the first set of attributes is identified as F;
an entity possessing the second set of attributes is identified as M;
d(F, M) is a Genetic Responsivity Measurement between F and M;
N is a number of attributes;
F₁, F₂, . . . , F_{N} represent a plurality of attribute values for F;
M₁, M₂, . . . , M_{N} represent a plurality of attribute values for M;
∥ denotes absolute value; and
wₙ is a number between 0 and 1 which assigns a weight to an n^{th} attribute.

36. A method according to claim 35, in which a value for a relative weight is computed using the expression:
$${w}_{n} = \frac{{W}_{n}}{{\sum }_{k = 1}^{N} {W}_{k}}$$
where Wₙ is an initially chosen weight for the n^{th} attribute.

37. A method according to claim 33, wherein a Genetic Responsivity Measurement from a first desired attribute set to a second existing attribute set is computed using the expression:
$$d ( FD , ME ) = {\sum }_{n = 1}^{N} {w}_{n} \vert {FD}_{n} - {ME}_{n} \vert$$
where FD is a desired attribute set of a first individual;
ME is an existing attribute set of a second individual;
FD₁, FD₂, . . . , FD_{N} represent a plurality of desired attribute values for F;
ME₁, ME₂, . . . , ME_{N} represent a plurality of existing attribute values for M;
∥ denotes absolute value; and
wₙ is a number between 0 and 1 which assigns a weight to an n^{th} attribute.

38. A method according to claim 32, wherein a bidirectional match is computed using the expression:
$${d}_{BDE} ( F , M ) = \frac{d ( FD , ME ) + d ( MD , FE )}{2}$$
where FD is a desired attribute of a first individual;
ME is an existing attribute of a second individual;
MD is a desired attribute of said second individual; and
FE is an existing attribute of said first individual.

39. A method according to claim 33, wherein a relative weight wₙ for all attributes is computed using the expression:
$${w}_{n} = \{ \begin{matrix}1 & if n ∈ R \\ \frac{{W}_{n}}{{\sum }_{k ∈ A - R} {W}_{k}} & if n ∈ A - R\end{matrix}$$
where ε means “is contained in”; and
A−R denotes a set of attributes that are in A but not in R.

40. A method according to claim 33, wherein the Genetic Responsivity Measurement is converted to a correlation using the expression
c(F,M)=1−d(F,M).

41. A method according to claim 31, wherein the data, identifiers of the first set are sources of information (S1-S{N}) of a master set (MS) and the data identifiers of the second set are individual attributes (54A-54D) collectively describing the target (56), and wherein the relevance Genetic Responsivity Measurements (58) computed in the computing step comprise a plurality of individual Genetic Responsivity Measurements (58A, 58B, 58C, 58D) between each of said individual attributes (54A, 54B, 54C . . . 54D) and said plurality of sources of information (S1, S2, S3, S4, S5, S6, S7, . . . S{N}).

42. A method according to claim 41, further comprising the steps of:
selecting a subset of the sources of information (S1′, S2′, S3′) in the general order of shortest relevance Genetic Responsivity Measurements (58) measured between said subset of sources of information and said set of attributes (54).

43. A method according to claim 41, further comprising the step of:
performing a page rank search plurality of individual attributes (54A, 54B, 54C . . . 54D) to reduce the total number of sources of information (S1, S2, S3, S4, S5, S6, S7, . . . S{N}) prior to applying the Genetic Responsivity Measurement formula to measure said relevance Genetic Responsivity Measurements (58).

44. A method according to claim 40, further comprising the steps of:
identifying non-relevant linked attributes; and
excluding from the search sources of information containing said non-relevant linked attributes.

45. A method according to claim 31, wherein the at least one sources of information include a web page.

46. A method according to claim 31, wherein the sets of data identifiers are encoded into respective vectors and the computing step and the subsequent steps comprise:
evaluation of the metric between each vector and other vectors; and decoding the metric.

47. A method according to claim 46, wherein the metric used is a sum of N components, where N is the dimension of the encoded vector space, each component being a specific function of a pair of corresponding values of two vectors.

48. A method according to claim 46, where the metrics are weighted using an N×N weighting matrix.

49. An apparatus for searching for a match, comprising:
means for storing a first set of attribute values (Fₙ) of at least one first entity (F, S);
means for inputting a user-selected second set of attribute values (Mₙ, 54) relating to a second entity (M, 56);
means for storing a Genetic Responsivity Measurement formula which is a function of the degree of match between respective values of the sets of attribute values; and
means for applying the formula to the first and second sets to determine an overall degree of match between the first entity and the second entity.

50. An apparatus as recited in claim 49, in which said first set of attribute values includes descriptive functional words.

51. A method comprising the steps of:
providing a website; said website being accessible to a plurality of persons; said plurality of persons including a plurality of women and a plurality of men;
receiving a plurality of sets of personal characteristics from said plurality of persons via said website; said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to one of said plurality of persons who provided said sample upon receiving a request from said same one of said plurality of persons; and
providing a relationship prediction to one of said plurality of persons; said relationship prediction being based on a combination of both a positive correlation of said first and said second sets of personal characteristics, and a measured dissimilarity between the sequence of genetic characteristics of a pair of said samples.

52. A method as recited in claim 51, in which said website includes a guide for helping said plurality of persons to provide said first and said second set of personal characteristics.

53. A method as recited in claim 51, in which said sample is obtained from a skin patch.

54. A method as recited in claim 51, in which said sample is obtained from saliva.

55. A method as recited in claim 51, in which said sample is obtained from blood.

56. A method as recited in claim 51, in which said first and said second sets of personal characteristics include information that describe desirable personality attributes.

57. A method as recited in claim 51, in which said first and said second sets of personal characteristics include information that describes desirable physical attributes.

58. A method as recited in claim 51, in which said first and said second sets of personal characteristics include information that describes desirable educational attributes.

59. A method as recited in claim 51, in which said samples are received by U.S. Mail.

60. A method as recited in claim 51, in which said samples are collected at a physical site.

61. A method as recited in claim 51, in which said sequences of genetic characteristics include a sequence of genetic information from the Major Histocompatibility Complex.

62. A method as recited in claim 51, in which said sequences of genetic characteristics include a sequence of genetic information from the DRD4 locus.

63. A method as recited in claim 51, in which said sequences of genetic characteristics include a sequence of genetic information from the AVPR1a locus.

64. A method as recited in claim 51, in which said sequences of genetic characteristics include a sequence of genetic information from the CD38 locus.

65. A method as recited in claim 51, in which said sequences of genetic characteristics include a sequence of genetic information from the ERα-locus.

66. A method as recited in claim 51, comprising the additional step of:
increasing the chances that an offspring of one of said plurality of women and one of said plurality of men will be healthy.

67. A method as recited in claim 51, further comprising the step of:
reducing the risk producing a child with a birth defects that may arise from one of said plurality of women and one of said plurality of men who are too closely related, and who may carry the same deleterious recessive gene.

68. A method as recited in claim 51, further comprising the step of:
increasing the fertility of one of said plurality of women and one of said plurality of men.

69. A method as recited in claim 51, further comprising the step of:
increasing the likelihood of reproductive success for one of said plurality of women and one of said plurality of men.

70. A method as recited in claim 51, further comprising the step of:
enhancing the immune system diversity of an offspring of one of said plurality of women and one of said plurality of men.

71. A method as recited in claim 51, further comprising the step of:
increasing the stability of a marriage between one of said plurality of women and one of said plurality of men.

72. A method as recited in claim 51, further comprising the step of:
enabling a subscriber to an online dating service to predict one of said plurality of women's attraction to one of said plurality of men, thus improving the chances of a compatible match.

73. A method as recited in claim 51, further comprising the step of:
enabling an individual to compare their own genome to another's genome with the goal of entering into satisfying and lasting relationship.

74. A method as recited in claim 51, further comprising the step of:
enabling one of said plurality of women and one of said plurality of men, who are considering a long-term relationship, to assess the probable stability of said relationship and the prospective health of their offspring.

75. A method as recited in claim 51, further comprising the step of: reducing the likelihood that one of said plurality of women will suffer a miscarriage.

76. A method as recited in claim 51, further comprising the step of:
reducing fetal loss for one of said plurality of women and one of said plurality of men.

77. A method as recited in claim 51, further comprising the step of:
reducing the chance that one of said plurality of women will suffer preeclampsia in her pregnancy.

78. A method, comprising the steps of:
providing a relationship prediction service to an organization for a fee paid by an organization;
sending a sample collection kit to each of a plurality of persons; said persons being affiliated with said organization;
receiving a plurality of samples from a portion of said plurality of persons;
analyzing each of a plurality of said samples which have been received to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person;
providing a relationship prediction to said organization; said relationship prediction being based on a combination of both a positive correlation of said first and said second sets of personal characteristics and a measured dissimilarity between the sequence of genetic characteristics of a pair of said samples; and
furnishing said relationship prediction to the person who provided said sample.

79. A method as recited in claim 78, in which said organization is an online dating service.

80. A method as recited in claim 78, in which said organization is a resort.

81. A method as recited in claim 78, in which said organization is a club.

82. A method as recited in claim 78, in which said organization is a church.

83. A method as recited in claim 78, in which said organization is a cruise line.

84. A method as recited in claim 78, in which said organization is a casino.

85. A method as recited in claim 78, in which said samples are received by a test facility.

86. A method as recited in claim 78, in which said samples are collected at a sample collection site.

87. A method as recited in claim 78, in which said sample is obtained from a skin patch.

88. A method as recited in claim 78, in which said sample is obtained from saliva.

89. A method as recited in claim 78, in which said sample is obtained from blood.

90. A method as recited in claim 78, in which said first and said second sets of personal characteristics include information that describe desirable personality attributes.

91. A method as recited in claim 78, in which said first and said second sets of personal characteristics include information that describe desirable physical attributes.

92. A method as recited in claim 78, in which said first and said second sets of personal characteristics include information that describe desirable educational attributes.

93. A method as recited in claim 78, in which said samples are received by a postal service.

94. A method as recited in claim 78, in which said samples are collected at a physical site.

95. A method as recited in claim 78, in which said sequences of genetic characteristics include a sequence of genetic information from the Major Histocompatibility Complex.

96. A method as recited in claim 78, further comprising the steps of:
storing said person's set of genetic attributes and said set of genetic attributes for a member of the opposite sex in a personal radio device; and
using a personal radio to find a match.

97. A method, comprising the steps of:
providing a relationship prediction service to a matchmaker for a fee paid by said matchmaker;
sending a sample collection kit to each of a plurality of persons; said persons being customers of said matchmaker;
receiving a plurality of samples from a portion of said plurality of persons;
analyzing each of a plurality of said samples which have been received to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person;
providing a relationship prediction to said matchmaker; said relationship prediction being based on a combination of both a positive correlation of said first and said second sets of personal characteristics and a measured dissimilarity between the sequence of genetic characteristics of a pair of said samples; and
furnishing said relationship prediction to the person who provided said sample.

98. A method comprising the steps of:
providing a website; said website being accessible to a plurality of persons;
receiving a plurality of sets of personal characteristics from said plurality of persons via said website; said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person; and
providing a relationship prediction to one of said plurality of individuals; said relationship prediction being based on a combination of both a positive correlation of said first and said second sets of personal characteristics, and a measured responsivity of a female to a male based upon the comparison of genetic characteristics.

99. A method, comprising the steps of:
providing a website; said website being accessible to a plurality of persons; said plurality of persons including a plurality of women and a plurality of men;
receiving a plurality of sets of personal characteristics from said plurality of persons via said website; said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person; and
providing a relationship prediction to one of said plurality persons; said relationship prediction being based on a combination of both a positive correlation of said first and said second sets of personal characteristics, and a measured dissimilarity between the sequence of genetic characteristics of a pair of said samples.

100. A method, comprising the steps of:
providing a database; said database containing data concerning a plurality of persons; said plurality of persons including a plurality of women and a plurality of men;
said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person; and
providing a relationship prediction to one of said plurality of persons; said relationship prediction being based on a combination of both a positive correlation of said first and said second sets of personal characteristics, and a measured dissimilarity between the sequence of genetic characteristics of a pair of said samples.

101. A method comprising the steps of:
providing a website; said website being accessible to a plurality of persons; said plurality of persons including a plurality of women and a plurality of men;
receiving a plurality of sets of personal characteristics from said plurality of persons via said website; said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person;
providing a relationship prediction to one of said plurality of persons; said relationship prediction being based on a combination of both a positive correlation of said first and said second sets of personal characteristics, and a measured dissimilarity between the sequence of genetic characteristics of a pair of said samples; and
providing additional instructions to the person who provided said sample if said person who provided said sample is using hormonal birth control.

102. A method, comprising the steps of:
providing a database; said database containing data concerning a plurality of persons; said plurality of persons including a plurality of women and a plurality of men;
said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person;
providing a relationship prediction to one of said plurality persons; said relationship prediction being based on a combination of both a positive correlation of said first and said second sets of personal characteristics, and a measured dissimilarity between the sequence of genetic characteristics of a pair of said samples; and
providing additional instructions to the person who provided said sample if said person who provided said sample is using hormonal birth control.

103. A method, comprising the steps of:
providing an introduction service; said introduction service for maintaining a database containing data concerning a plurality of persons; said plurality of persons including a plurality of women and a plurality of men;
said sets of personal characteristics including a first set of personal characteristics which describe one of said persons who submit a first set of personal characteristics, and a second set of personal characteristics which describe a perceived match for another person;
collecting a sample from a portion of each of said plurality of persons;
analyzing each of a plurality of said samples which have been collected to determine a sequence of genetic characteristics for each of said plurality of samples;
storing the results of the analysis of said plurality of samples;
providing a copy of one of said sequences of genetic characteristics for one of said samples to the person who provided said sample upon receiving a request from the same person; and
providing a relationship prediction to one of said plurality persons; said relationship prediction being based on a combination of both a positive correlation of said first and said second sets of personal characteristics, and a measured dissimilarity between the sequence of genetic characteristics of a pair of said samples.

104. A composition of matter, comprising:
an active ingredient (206);
a base (208);
said active ingredient (206) being added to said base (208) to compose a mixture;
said active ingredient (206) including a biological agent;
said biological agent being selected to match a genetic attribute possessed by a person.

105. A composition of matter as recited in claim 104, in which said base (208) is a solvent.

106. A composition of matter as recited in claim 104, in which said base (208) is alcohol.

107. A composition of matter as recited in claim 104, in which said base (208) is water.

108. A composition of matter as recited in claim 104, in which said biological agent is a peptide.

109. A composition of matter as recited in claim 104, in which said peptide is selected from a group of peptides related to a cluster of human genes called the Major Histocompatibility Complex.

110. A composition of matter as recited in claim 104, in which said peptide is selected to promote the responsivity of a person using said mixture.

111. A composition of matter as recited in claim 104, in which said peptide is selected to promote the responsivity of a person to another person using said mixture.

112. A composition of matter as recited in claim 104, in which said peptide is selected to promote the confidence of a person using said mixture.

113. A composition of matter as recited in claim 104, in which said peptide is selected to promote the interest of another person using said mixture.

114. A composition of matter as recited in claim 104, in which said peptide is selected to promote the attraction of a person using said mixture.

115. A composition of matter as recited in claim 104, in which said mixture is fabricated as a perfume.

116. A composition of matter as recited in claim 104, in which said mixture is fabricated as a cologne.

117. A composition of matter as recited in claim 104, in which said mixture is fabricated as a salve.

118. A composition of matter as recited in claim 104, in which said mixture is fabricated as an aerosol spray.

119. A composition of matter as recited in claim 104, in which said mixture is incorporated in a cosmetic.

120. A composition of matter as recited in claim 119, in which said cosmetic is a lipstick.

121. A composition of matter as recited in claim 104, in which said mixture is applied to the skin.

122. A composition of matter as recited in claim 104, in which said mixture is applied to an article of clothing.

123. A composition of matter as recited in claim 104, in which said mixture is contained in an air freshener.

124. A composition of matter as recited in claim 104, in which said mixture is contained in an aroma-dispensing device.

125. A composition of matter as recited in claim 104, in which said mixture is contained in a candle.

126. A composition of matter as recited in claim 104, in which said mixture is contained in a piece of incense.

127. A perfume, comprising:
a base (208); and
a plurality of active ingredients (206); said plurality of active ingredients (206) being mixed with said base (208);
said plurality of active ingredients (206) being selected based base (54) on an analysis of a human sample; said human sample containing information regarding a genetic analysis of a user from whom said sample was obtained;
said plurality of active ingredients (206) being further selected to elicit a sexual response.

128. A perfume as recited in claim 127, which is worn by said user, from whom said human sample was obtained.

129. A perfume as recited in claim 127, which is worn by said user, from whom said human sample was obtained, to broadcast sexual compatibility.

130. A perfume as recited in claim 127, which is worn by another person designated by said user, from whom said human sample was obtained.

131. A perfume as recited in claim 128, which is worn by another person designated by said user, from whom said human sample was obtained, to broadcast sexual compatibility.

132. A perfume as recited in claim 127, which is contained in a medication that is taken internally, and which is then secreted through the skin.

133. A perfume as recited in claim 127, which is contained in a medication that is taken internally, and then which causes a biological reaction which produces an odor.

134. A method, comprising the steps of:
predicting a good relationship; and
advising a woman that a man may find her less attractive if she has started using hormonal birth control.

135. A method, comprising the steps of:
predicting a good relationship; and
advising a woman that a man may find her less attractive if she has stopped using hormonal birth control.

136. A method, comprising the steps of:
operating a website;
enabling a customer to access said website;
allowing said customer to request advice concerning maintaining a good relationship; and
providing advice to said customer;
said advice including a recommendation that a man may find her less attractive if she has started using hormonal birth control.

137. A method, comprising the steps of:
operating a website;
enabling a customer to access said website;
allowing said customer to request advice concerning maintaining a good relationship; and
providing advice to said customer;
said advice including a recommendation that a man may find her less attractive if she has stopped using hormonal birth control.

138. A method, comprising the steps of:
operating a website;
enabling a customer to access said website;
asking a female customer if she has started using hormonal birth control;
asking a female customer if she believes that her male mate finds her less attractive since she changed her hormonal birth control;
correlating her responses; and
providing advice based on said responses to other female customers accessing said website as a public service.

139. A method, comprising the steps of:
operating a website;
enabling a customer to access said website;
asking a female customer if she has stopped using hormonal birth control;
asking a female customer if she believes that her male mate finds her less attractive since she has stopped using her hormonal birth control;
correlating her responses; and
providing advice based on said responses to other female customers accessing said website as a public service.

140. A method, comprising the steps of:
providing a website; said website being accessible to a plurality of persons; said plurality of persons including a plurality of women and a plurality of men;
receiving a set of personal characteristics from a woman via said website; said set of personal characteristics including a first set of personal characteristics which describe said woman, who submits a first set of personal characteristics, and a second set of personal characteristics which describe a suitable man;
collecting a plurality of samples from said woman and from said plurality of men;
analyzing said plurality of samples to determine a plurality of sequences of genetic characteristics;
storing the results of the analysis of said plurality of samples;
providing a copy sequence of genetic characteristics of said sample to said woman who provided said sample upon receiving a request from said woman; and
providing a relationship prediction to said woman; said relationship prediction being based on a combination of both a positive correlation of said first and said second sets of personal characteristics, and a measured dissimilarity between the sequence of genetic characteristics of a pair of said plurality of samples.

141. A method as recited in claim 140, further comprising the step of:
advising a woman that a man may find her less attractive if she has started using hormonal birth control.

142. A method as recited in claim 140, further comprising the step of:
advising a woman that a man may find her less attractive if she has stopped using hormonal birth control.

143. A method, comprising the steps of:
obtaining a sample from a first person;
obtaining a sample from a second person;
analyzing both of said first and said second samples to produce a first and a second genetic sequence;
comparing said first and said second genetic sequences;
predicting a match between said first person and said second person based on the comparison of said first and said second genetic sequences; and
producing a graphic representation of said match;
said graphic representation of said match illustrating the quality of said match.

144. A method as recited in claim 143, further comprising the step of:
furnishing said first and said second samples to a laboratory for analysis;
said laboratory providing one of said first and said second persons with an alphanumeric code which describes their own genetic sequences.

145. A method as recited in claim 144, further comprising the step of:
comparing a code with that of another person to estimate mutual compatibility and quality of a potential romantic relationship.

146. A method as recited in claim 145, further comprising the step of:
comparing a code with that of another person to estimate mutual compatibility and quality of a potential romantic relationship.

147. A method as recited in claim 146, further comprising the step of:
providing a written guide for comparing the data embedded in a code to estimate the quality of a contemplated relationship.

148. A method as recited in claim 146, further comprising the step of:
providing a computer program for comparing the data embedded in a code to estimate the quality of a contemplated relationship.

149. A method as recited in claim 148, further comprising the step of:
keeping a record of said genetic sequence at said laboratory.

150. A method as recited in claim 149, further comprising the steps of:
comparing said first and said second genetic sequences at said laboratory; and
issuing a report.

151. A method as recited in claim 150, wherein said report includes a compatibility score which rates the predicted quality of a contemplated relationship.
